SEQUENCE LISTING (109 sequences)

SEQ ID NO: 1 - protein, 6 aa, Artificial Sequence
  Description of Artificial Sequence: Synthetic peptide
KVKVLG

SEQ ID NO: 2 - protein, 6 aa, Artificial Sequence
  Description of Artificial Sequence: Synthetic peptide
GDVIEV

SEQ ID NO: 3 - protein, 11 aa, Artificial Sequence
  Description of Artificial Sequence: Synthetic peptide
KVKVLGDVIEV

SEQ ID NO: 4 - protein, 11 aa, Artificial Sequence
  Description of Artificial Sequence: Synthetic peptide
  MOD_RES (2)..(2): (2-bromoallyl)-Gly
KGKVLGDVIEV

SEQ ID NO: 5 - protein, 11 aa, Artificial Sequence
  Description of Artificial Sequence: Synthetic peptide
  MOD_RES (8)..(8): (2-bromoallyl)-Gly
KVKVLGDGIEV

SEQ ID NO: 6 - protein, 11 aa, Artificial Sequence
  Description of Artificial Sequence: Synthetic peptide
KLKVLGDVIEV

SEQ ID NO: 7 - protein, 25 aa, Artificial Sequence
  Description of Artificial Sequence: Synthetic peptide
GKVKVLGDVIEVGGKVKVLGDVIEV

SEQ ID NO: 8 - protein, 25 aa, Artificial Sequence
  Description of Artificial Sequence: Synthetic peptide
GKLKVLGDVIEVGGKLKVLGDVIEV

SEQ ID NO: 9 - protein, 25 aa, Artificial Sequence
  Description of Artificial Sequence: Synthetic peptide
GKLKWLGDVIEVGGKLKWLGDVIEV

SEQ ID NO: 10 - protein, 7 aa, Artificial Sequence
  Description of Artificial Sequence: Synthetic peptide
GNNQQNY

SEQ ID NO: 11 - skipped

SEQ ID NO: 12 - protein, 7 aa, Artificial Sequence
  Description of Artificial Sequence: Synthetic peptide
GNNQQNY

SEQ ID NO: 13 - protein, 12 aa, Artificial Sequence
  Description of Artificial Sequence: Synthetic peptide
GKLKVLGDVIEV

SEQ ID NO: 14 - protein, 25 aa, Artificial Sequence
  Description of Artificial Sequence: Synthetic peptide
GKLKVLGDVIEVPGKLKVLGDVIEV

SEQ ID NO: 15 - protein, 27 aa, Artificial Sequence
  Description of Artificial Sequence: Synthetic peptide
GKLKVLGDVIEVGGGGKLKVLGDVIEV

SEQ ID NO: 16 - protein, 24 aa, Artificial Sequence
  Description of Artificial Sequence: Synthetic peptide
KLKVLGDVIEVGGKLKVLGDVIEV

SEQ ID NO: 17 - protein, 25 aa, Artificial Sequence
  Description of Artificial Sequence: Synthetic peptide
GKVKVLGDVIEVGGKVKVLGDVIEV

SEQ ID NO: 18 - protein, 25 aa, Artificial Sequence
  Description of Artificial Sequence: Synthetic peptide
GKLKVNGDVIEVGGKLKVNGDVIEV

SEQ ID NO: 19 - protein, 25 aa, Artificial Sequence
  Description of Artificial Sequence: Synthetic peptide
GKLKVLPDVIEVGGKLKVLPDVIEV

SEQ ID NO: 20 - protein, 25 aa, Artificial Sequence
  Description of Artificial Sequence: Synthetic peptide
GKLKRLGDDIEVGGKLKDLGDRIEV

SEQ ID NO: 21 - protein, 25 aa, Artificial Sequence
  Description of Artificial Sequence: Synthetic peptide
GVGSNKGAIIGLGGVGSNKGAIIGL

SEQ ID NO: 22 - protein, 25 aa, Artificial Sequence
  Description of Artificial Sequence: Synthetic peptide
GSNKGAIIGLMVGGSNKGAIIGLMV

SEQ ID NO: 23 - protein, 25 aa, Artificial Sequence
  Description of Artificial Sequence: Synthetic peptide
GKGAIIGLMVGGGGKGAIIGLMVGG

SEQ ID NO: 24 - protein, 25 aa, Artificial Sequence
  Description of Artificial Sequence: Synthetic peptide
GAIIGLMVGGVVGGAIIGLMVGGVV

SEQ ID NO: 25 - protein, 25 aa, Artificial Sequence
  Description of Artificial Sequence: Synthetic peptide
GIGLMVGGVVIAGGIGLMVGGVVIA

SEQ ID NO: 26 - protein, 33 aa, Artificial Sequence
  Description of Artificial Sequence: Synthetic polypeptide
GKGAIIGLMVGGVVIAGGKGAIIGLMVGGVVIA

SEQ ID NO: 27 - protein, 25 aa, Artificial Sequence
  Description of Artificial Sequence: Synthetic peptide
GLVFFAGDVGSNGGLVFFAGDVGSN

SEQ ID NO: 28 - protein, 23 aa, Artificial Sequence
  Description of Artificial Sequence: Synthetic peptide
GLVFFAGDVGSNKGAIIGLMVGG

SEQ ID NO: 29 - protein, 27 aa, Artificial Sequence
  Description of Artificial Sequence: Synthetic peptide
GLVFFAGDVGSNKGAIIGLMVGGVVIA

SEQ ID NO: 30 - protein, 23 aa, Artificial Sequence
  Description of Artificial Sequence: Synthetic peptide
GKLVFFADVGSNKGAIIGLMVGG

SEQ ID NO: 31 - protein, 27 aa, Artificial Sequence
  Description of Artificial Sequence: Synthetic peptide
GKLVFFADVGSNKGAIIGLMVGGVVIA

SEQ ID NO: 32 - protein, 24 aa, Artificial Sequence
  Description of Artificial Sequence: Synthetic peptide
GKLVFFAENVGSNKGAIIGLMVGG

SEQ ID NO: 33 - protein, 28 aa, Artificial Sequence
  Description of Artificial Sequence: Synthetic peptide
GKLVFFAENVGSNKGAIIGLMVGGVVIA

SEQ ID NO: 34 - protein, 44 aa, Artificial Sequence
  Description of Artificial Sequence: Synthetic polypeptide
GAIIGLMVGGVVIAGPAIIGLMVGGVVIAGPAIIGLMVGGVVIA

SEQ ID NO: 35 - protein, 44 aa, Artificial Sequence
  Description of Artificial Sequence: Synthetic polypeptide
GAIIGLMVGPVVIAGPAIIGLMVGPVVIAGPAIIGLMVGPVVIA

SEQ ID NO: 36 - protein, 89 aa, Artificial Sequence
  Description of Artificial Sequence: Synthetic polypeptide
GAIIGLMVGGVVIAGPAIIGLMVGGVVIAGPAIIGLMVGGVVIAGPAIIGLMVGGVVIAGPAIIGLMVGGVVIAGPAIIGLMVGGVVIA

SEQ ID NO: 37 - protein, 10 aa, Artificial Sequence
  Description of Artificial Sequence: Synthetic peptide
  N-term Ac
  C-term NH2
AGDVGSNKGA

SEQ ID NO: 38 - protein, 22 aa, Artificial Sequence
  Description of Artificial Sequence: Synthetic peptide
  N-term Ac
  C-term NH2
AGDVGSNKGAGGAGDVGSNKGA

SEQ ID NO: 39 - protein, 11 aa, Artificial Sequence
  Description of Artificial Sequence: Synthetic peptide
  N-term Ac
  C-term NH2
VGSNKGAIIGL

SEQ ID NO: 40 - protein, 24 aa, Artificial Sequence
  Description of Artificial Sequence: Synthetic peptide
  N-term Ac
  C-term NH2
VGSNKGAIIGLGGVGSNKGAIIGL

SEQ ID NO: 41 - protein, 11 aa, Artificial Sequence
  Description of Artificial Sequence: Synthetic peptide
  N-term Ac
  C-term NH2
GSNKGAIIGLM

SEQ ID NO: 42 - protein, 24 aa, Artificial Sequence
  Description of Artificial Sequence: Synthetic peptide
  N-term Ac
  C-term NH2
GSNKGAIIGLMGGGSNKGAIIGLM

SEQ ID NO: 43 - protein, 11 aa, Artificial Sequence
  Description of Artificial Sequence: Synthetic peptide
GSNKGAIIGLM

SEQ ID NO: 44 - protein, 11 aa, Artificial Sequence
  Description of Artificial Sequence: Synthetic peptide
  N-term Ac
  C-term NH2
SNKGAIIGLMV

SEQ ID NO: 45 - protein, 42 aa, Artificial Sequence
  Description of Artificial Sequence: Synthetic polypeptide
DAEFRHDSGYEVHHQKLVFFAEDVGSNKVAIIGLMVGGVVIA

SEQ ID NO: 46 - protein, 42 aa, Artificial Sequence
  Description of Artificial Sequence: Synthetic polypeptide
DAEFRHDSGYEVHHQKLVFFAEDVGSNKGAIIVLMVGGVVIA

SEQ ID NO: 47 - protein, 42 aa, Artificial Sequence
  Description of Artificial Sequence: Synthetic polypeptide
DAEFRHDSGYEVHHQKLVFFAEDVGSNKGAIIGLMVVGVVIA

SEQ ID NO: 48 - protein, 42 aa, Artificial Sequence
  Description of Artificial Sequence: Synthetic polypeptide
DAEFRHDSGYEVHHQKLVFFAEDVGSNKGAIIGLMVGVVVIA

SEQ ID NO: 49 - protein, 25 aa, Artificial Sequence
  Description of Artificial Sequence: Synthetic peptide
GSSNNFGAILSSGGSSNNFGAILSS

SEQ ID NO: 50 - protein, 37 aa, Artificial Sequence [sequence omitted]

SEQ ID NO: 51 - protein, 37 aa, Artificial Sequence
  Description of Artificial Sequence: Synthetic polypeptide
KCNTATCATQRLANFLVHSSNNFVAILSSTNVGSNTY

SEQ ID NO: 52 - protein, 37 aa, Artificial Sequence
  Description of Artificial Sequence: Synthetic polypeptide
KCNTATCATQRLANFLVHSSNNFGAILSSTNVVSNTY

SEQ ID NO: 53 - protein, 37 aa, Artificial Sequence
  Description of Artificial Sequence: Synthetic polypeptide
KCNTATCATQRLANFLVHSSNNFVAILSSTNVVSNTY

SEQ ID NO: 54 - protein, 25 aa, Artificial Sequence
  Description of Artificial Sequence: Synthetic peptide
GAAAGAVVGGLGGGAAAGAVVGGLG

SEQ ID NO: 55 - protein, 25 aa, Artificial Sequence
  Description of Artificial Sequence: Synthetic peptide
GAVVGGLGGYMLGGAVVGGLGGYML

SEQ ID NO: 56 - protein, 24 aa, Artificial Sequence
  Description of Artificial Sequence: Synthetic peptide
GLGGYMLGSAMGGGLGGYMLGSAM

SEQ ID NO: 57 - protein, 25 aa, Artificial Sequence
  Description of Artificial Sequence: Synthetic peptide
GTTTTKGENFTEGGTTTTKGENFTE

SEQ ID NO: 58 - protein, 25 aa, Artificial Sequence
  Description of Artificial Sequence: Synthetic peptide
GMKHMAGAAAAGAVVGGLGGYMLGS

SEQ ID NO: 59 - protein, 23 aa, Artificial Sequence
  Description of Artificial Sequence: Synthetic peptide
GAAAAGAVVGGLGGYMLGSAMSR

SEQ ID NO: 60 - protein, 11 aa, Artificial Sequence
  Description of Artificial Sequence: Synthetic peptide
PVKVWGSIKGL

SEQ ID NO: 61 - protein, 11 aa, Artificial Sequence
  Description of Artificial Sequence: Synthetic peptide
KVKVWGSIKGL

SEQ ID NO: 62 - protein, 25 aa, Artificial Sequence
  Description of Artificial Sequence: Synthetic peptide
GKVWGSIKGLTEGGKVWGSIKGLTE

SEQ ID NO: 63 - protein, 25 aa, Artificial Sequence
  Description of Artificial Sequence: Synthetic peptide
GWGSIKGLTEGLGGWGSIKGLTEGL

SEQ ID NO: 64 - skipped

SEQ ID NO: 65 - protein, 11 aa, Artificial Sequence
  Description of Artificial Sequence: Synthetic peptide
KVWVSIKGLTE

SEQ ID NO: 66 - protein, 11 aa, Artificial Sequence
  Description of Artificial Sequence: Synthetic peptide
KVWGSIKVLTE

SEQ ID NO: 67-68 - skipped (2 entries)

SEQ ID NO: 69 - protein, 25 aa, Artificial Sequence
  Description of Artificial Sequence: Synthetic peptide
GVTNVGGAVVTGGGVTNVGGAVVTG

SEQ ID NO: 70 - protein, 24 aa, Artificial Sequence
  Description of Artificial Sequence: Synthetic peptide
GAVVTGVTAVAGGGAVVTGVTAVA

SEQ ID NO: 71 - protein, 29 aa, Artificial Sequence
  Description of Artificial Sequence: Synthetic peptide
GEGVVHGVATVAEKTKEQVTNVGGAVVTG

SEQ ID NO: 72 - protein, 30 aa, Artificial Sequence
  Description of Artificial Sequence: Synthetic polypeptide
GVTNVGGAVVTGVTAVAQKTVEGAGSIAAA

SEQ ID NO: 73 - protein, 25 aa, Artificial Sequence
  Description of Artificial Sequence: Synthetic peptide
GVTNVGGAVVTGNGTVEGAGSIAAA

SEQ ID NO: 74 - protein, 31 aa, Artificial Sequence
  Description of Artificial Sequence: Synthetic polypeptide
GAVVTGVTAVAQKTVEGAGSIAAATGFVKKD

SEQ ID NO: 75 - protein, 11 aa, Artificial Sequence
  Description of Artificial Sequence: Synthetic peptide
IAAATGFVKKD

SEQ ID NO: 76 - protein, 25 aa, Artificial Sequence
  Description of Artificial Sequence: Synthetic peptide
GRKDQGGYTMHQGGRKDQGGYTMHQ

SEQ ID NO: 77 - protein, 25 aa, Artificial Sequence
  Description of Artificial Sequence: Synthetic peptide
GHQDQEGDTDAGGGHQDQEGDTDAG

SEQ ID NO: 78 - protein, 25 aa, Artificial Sequence
  Description of Artificial Sequence: Synthetic peptide
GEDEAAGHVTQAGGEDEAAGHVTQA

SEQ ID NO: 79 - protein, 25 aa, Artificial Sequence
  Description of Artificial Sequence: Synthetic peptide
GAKGADGKTKIAGGAKGADGKTKIA

SEQ ID NO: 80 - protein, 25 aa, Artificial Sequence
  Description of Artificial Sequence: Synthetic peptide
GAKGADGKTKITGGAKGADGKTKIT

SEQ ID NO: 81 - protein, 11 aa, Artificial Sequence
  Description of Artificial Sequence: Synthetic peptide
AKGADGKTKIA

SEQ ID NO: 82 - protein, 11 aa, Artificial Sequence
  Description of Artificial Sequence: Synthetic peptide
AKGADGKTKIT

SEQ ID NO: 83 - protein, 25 aa, Artificial Sequence
  Description of Artificial Sequence: Synthetic peptide
GSPVVSGDTSPRGGSPVVSGDTSPR

SEQ ID NO: 84 - protein, 25 aa, Artificial Sequence
  Description of Artificial Sequence: Synthetic peptide
GNVSSTGSIDMVGGNVSSTGSIDMV

SEQ ID NO: 85 - protein, 11 aa, Artificial Sequence
  Description of Artificial Sequence: Synthetic peptide
DLIIKGISVHI

SEQ ID NO: 86 - protein, 12 aa, Artificial Sequence
  Description of Artificial Sequence: Synthetic peptide
DLIIKGISVHIE

SEQ ID NO: 87 - protein, 24 aa, Artificial Sequence
  Description of Artificial Sequence: Synthetic peptide
GAMMAAAQAALQSSWGMMGMLASQ

SEQ ID NO: 88 - protein, 24 aa, Artificial Sequence
  Description of Artificial Sequence: Synthetic peptide
GAMMAAAQAALKSSWGMMGMLASQ

SEQ ID NO: 89 - protein, 24 aa, Artificial Sequence
  Description of Artificial Sequence: Synthetic peptide
GAMMAAAQAALQSSWGMVGMLASQ

SEQ ID NO: 90 - protein, 25 aa, Artificial Sequence
  Description of Artificial Sequence: Synthetic peptide
GSWGMMGMLASQGGMAAAQAALQSS

SEQ ID NO: 91 - protein, 25 aa, Artificial Sequence
  Description of Artificial Sequence: Synthetic peptide
GSWGMMGMLASQGGMAAAQAALKSS

SEQ ID NO: 92 - protein, 25 aa, Artificial Sequence
  Description of Artificial Sequence: Synthetic peptide
GSWGMVGMLASQGGMAAAQAALQSS

SEQ ID NO: 93 - protein, 31 aa, Artificial Sequence
  Description of Artificial Sequence: Synthetic polypeptide
GGSNMGGGMNFGAFSINPAMMAAAQAALQSS

SEQ ID NO: 94 - protein, 31 aa, Artificial Sequence
  Description of Artificial Sequence: Synthetic polypeptide
GGSNMGGGMNFGAFSINPAMMAAAQAALKSS

SEQ ID NO: 95 - protein, 31 aa, Artificial Sequence
  Description of Artificial Sequence: Synthetic polypeptide
GGSNMGGGMNFGTFSINPAMMAAAQAALQSS

SEQ ID NO: 96 - protein, 33 aa, Artificial Sequence
  Description of Artificial Sequence: Synthetic polypeptide
GGGMNFGAFSINPAMMAAAQAALQSSWGMMGML

SEQ ID NO: 97 - protein, 33 aa, Artificial Sequence
  Description of Artificial Sequence: Synthetic polypeptide
GGGMNFGAFSINPAMMAAAQAALKSSWGMMGML

SEQ ID NO: 98 - protein, 33 aa, Artificial Sequence
  Description of Artificial Sequence: Synthetic polypeptide
GGGMNFGAFSINPAMMAAAQAALQSSWGMVGML

SEQ ID NO: 99 - protein, 33 aa, Artificial Sequence
  Description of Artificial Sequence: Synthetic polypeptide
GGGMNFGTFSINPAMMAAAQAALQSSWGMMGML

SEQ ID NO: 100 - protein, 31 aa, Artificial Sequence
  Description of Artificial Sequence: Synthetic polypeptide
GSWGMMGMLASQQNQSGPSGNNQNQGNMQRE

SEQ ID NO: 101 - protein, 31 aa, Artificial Sequence
  Description of Artificial Sequence: Synthetic polypeptide
GSWGMVGMLASQQNQSGPSGNNQNQGNMQRE

SEQ ID NO: 102 - protein, 31 aa, Artificial Sequence
  Description of Artificial Sequence: Synthetic polypeptide
GSWGMMGMLASQQKQSGPSGNNQNQGNMQRE

SEQ ID NO: 103 - protein, 25 aa, Artificial Sequence
  Description of Artificial Sequence: Synthetic peptide
GSWGMMGMLASQGGSWGMMGMLASQ

SEQ ID NO: 104 - protein, 25 aa, Artificial Sequence
  Description of Artificial Sequence: Synthetic peptide
GSWGMVGMLASQGGSWGMVGMLASQ

SEQ ID NO: 105 - protein, 25 aa, Artificial Sequence
  Description of Artificial Sequence: Synthetic peptide
GLQSSWGMMGMLGGLQSSWGMMGML

SEQ ID NO: 106 - protein, 25 aa, Artificial Sequence
  Description of Artificial Sequence: Synthetic peptide
GLKSSWGMMGMLGGLKSSWGMMGML

SEQ ID NO: 107 - protein, 25 aa, Artificial Sequence
  Description of Artificial Sequence: Synthetic peptide
GLQSSWGMVGMLGGLQSSWGMVGML

SEQ ID NO: 108 - protein, 25 aa, Artificial Sequence
  Description of Artificial Sequence: Synthetic peptide
GDLIIKGISVHIGGDLIIKGISVHI

SEQ ID NO: 109 - protein, 25 aa, Artificial Sequence
  Description of Artificial Sequence: Synthetic peptide
GKVKVWGSIKGLGGKVKVWGSIKGL

(19) United States
(12) Patent Application Publication
(10) Pub. No.: US 2015/0307573 A1
(43) Pub. Date: Oct. 29, 2015

(54) CYLINDRINS AS ETIOLOGIC AGENTS OF AMYLOID DISEASES

(71) Applicant: The Regents of the University of California, Oakland, CA (US)

(72) Inventors: Arthur David Laganowsky, Los Angeles, CA (US); David S. Eisenberg, Los Angeles, CA (US); Rebecca Anne Villarama, Los Angeles, CA (US)

(21) Appl. No.: 14/373,629

(22) PCT Filed: Jan. 22, 2013
(86) PCT No.: PCT/US13/22574

§ 371 Date: Jul. 21, 2014

Related U.S. Application Data
(60) Provisional application No. 61/588,478, filed on Jan. 19, 2012.
(60) Provisional application No. 61/590,085, filed on Jan. 24, 2012.

(51) Int. Cl. C07K 14/47; G06F 19/12; A61K 38/00; C12N 9/02; G01N 33/50

(52) U.S. Cl. CPC C07K 14/4703; C12N 9/0089; G01N 33/5014; A61K 38/00; G06F 19/12; C12Y 115/01001; G01N 2333/4704; G01N 2333/90283

(57) ABSTRACT

This invention relates, e.g., to a cylindrin, which is a non-covalent assembly of substantially identical chains of an amyloid or amyloid-related protein, which is a non-covalent assembly of substantially identical chains of an amyloid or amyloid-related protein, wherein each chain has a length of about 10-100 amino acid residues and comprises
a single copy of a cylindrin-forming segment, or
tandem adjacent copies of a cylindrin-forming segment, optionally separated by spacers, or
adjacent copies of a first cylindrin-forming segment and a second complementary segment of the first cylindrin-forming segment, optionally separated by spacers,
wherein at least about ⅔ of the amino acid residues in the chain are cylindrin-forming segments,
   wherein the cylindrin is a curved beta sheet formed from anti-parallel out-of-register extended protein strands, which is substantially filled with packed side chains.

Also disclosed are methods for designing and making cylindrins, and methods for using them to identify inhibitors cylindrin-mediated cell toxicity.

This application claims the benefit of the filing date of U.S. Provisional Applications 61/588,478, filed Jan. 19, 2012, and 61/590,085, filed Jan. 24, 2012, each of which is incorporated by reference herein in its entirety.

This invention was made with Government support under Grant No. AG029430 awarded by the National Institutes of Health, Grant No. 0445429, awarded by the National Science Foundation, and grant No. DE-ACO2-06CH11357, awarded by United States Department of Energy. The Government has certain rights in this invention.

### BACKGROUND INFORMATION

Amyloid diseases, including Alzheimer's, Parkinson's, and the prion conditions, are each associated with a particular protein in fibrillar form. Studies from many laboratories have suggested that the molecular agents (toxic entities) in amyloid-related conditions are not the associated protein fibrils that have long been taken as the defining feature of these disorders, but instead are lower molecular weight entities, often termed “small amyloid oligomers” (1-7). These oligomers are not generally stable aggregates; they appear as transient species during the conversion of their monomeric precursors to more massive, stable fibrils, and sometimes they appear as an ensemble of sizes and shapes. This polymorphic and time-dependent nature of small amyloid oligomers has made it difficult to pin down their assembly pathways, their stoichiometries, their atomic-level structures, their relationship to fibrils, and their pathological actions (1, 8-10). What has emerged is a consensus, minimal definition of small amyloid oligomers: they are non-covalent assemblies of several identical chains of proteins known also to form amyloid fibrils; the oligomers exhibit greater cytotoxicity than either the monomer or fibrils formed from the same protein; in many cases the oligomer is recognized by a “conformational” antibody (A11) that binds oligomers but not fibrils, regardless of the sequence of the constituent protein (5). This suggests that oligomers display common conformation features that differ from those of fibrils (11).

There is a need to better define small amyloid oligomers which are important etiologic agents for amyloid diseases or conditions and/or to isolate artificial versions of them which mimic the properties of the small amyloid oligomers, in order to devise reagents and assays for identifying putative agents which reduce toxicity of the small amyloid oligomers. Such agents would be expected to be useful for treating diseases or conditions which are mediated by the small amyloid oligomers.

### DESCRIPTION OF THE DRAWINGS

The patent or application file contains at least one drawing executed in color. It is noted that many of these color drawings are present in the publication, Laganowsky et al. (2012), Atomic view of a toxic small oligomer, Science 335, 1228-31, doi: 10.1126.

FIG. 1 shows that the cylindrins derived from alphaB crystallin (ABC), an amyloid-forming protein, exhibit the properties of oligomeric state, immunoreactivity, and cytotoxicity commonly ascribed to small amyloid oligomers. (A) Ribbon diagram of a single subunit of ABC (16), colored by propensity to form amyloid, with red being the highest and blue the lowest propensity. The segment from residue 90 to 100, termed K11V, forms the cylindrin. (B) Representative electron micrograph of amyloid fibrils formed by the tandem repeat V2L variant of K11V, K11V-TR. (C) Overlaid size exclusion chromatograms showing protein standards (blued dashed curve) and cylindrin segments. K11VV2L (purple curve; 1.2 kDa) and K11V-TR (green curve; 2.5 kDa) cylindrin segments migrate as oligomeric complexes. A mutant form of K11V-TR that disrupts oligomer formation of the cylindrin peptide, K11VV4W-TR (orange curve; 2.7 kDa), migrates as a dimeric/monomeric species. (D) Native nanoelectrospray mass spectrum of K11V-TR peak fractions from SEC-HPLC reveals trimeric tandem repeat cylindrin oligomers, confirming that the oligomeric complexes coincide in mass with the crystallized cylindrins. Expansion of the most abundant ion series of a +5 charge state corresponding to a molecular mass of the three K11V-TR, coinciding with the crystallographic trimeric oligomer with a mass accuracy of 3.93 ppm is shown, with m/z labels. (E) Immuno-dot blot analysis of solutions of K11VV2L and K11V-TR, and K11V-TR fibrils with prefibrillar oligomer-specific, polyclonal antibody, A11 (5), and a mixture of fibril-specific monoclonal antibodies, OC (11). Solutions of cylindrin-forming segments are recognized by A11, whereas not by the OC antibody. In contrast, K11V-TR fibrils are recognized only by the OC antibody. Positive controls are shown to the right (5). (F) Cylindrin K11V-TR is toxic to four mammalian cells lines. Cell viability levels return to nearly 100% when we tested the control variant K11VV4W-TR. All samples were at a final concentration of 100 μM. Results represent mean±SEM. Student's t-test (N=4): **, P<0.01; and ***, P<0.001.

FIG. 2 shows crystal structures of cylindrins and computed free energy change of the simulated structural transition from cylindrin to a fibril. Each colored beta-strand (arrow) is composed of eleven amino acid residues from ABC of sequence KVKVLGDVIEV (K11V). (A) Schematic of unrolled cylindrin (outside view), illustrating strand-to-strand registration. Hydrogen bonds between the main chains of neighboring strands are shown by yellow dashed lines; hydrogen bonds mediated by water bridges or side chains are shown by blue dashed lines. (B) Ribbon representation of the cylindrin crystal structure. Pairs of strands form anti-parallel dimers, which assemble around a three-fold axis down the barrel axis of the cylindrin. The height of the cylindrin is 22 Å. The inner dimension of the cylindrin, around the waist from Ca to Ca, is 12 Å, and at the splayed ends is 22 Å. (C) The cylindrin with sidechains shown as atoms, and hydrogen bonds in yellow. Twelve backbone hydrogen bonds stabilize the strong interface between tightly twisted anti-parallel strands (e.g. between green and purple chains). The weaker interface between the pairs of tightly twisted strands is formed by four main-chain hydrogen bonds, with an additional two hydrogen bonds coming from a water bridge and two hydrogen bonds from side chain interactions (e.g. between purple and blue chains). The dry interior of the cylinder is closed by triplets of Val residues, shown as spheres, at the top and bottom. (D) Crystal structure of K11V-TR formed by three chains of 25 residues each. (E) Schematic of unrolled K11V-TR cylindrin (outside view). Similar hydrogen bonding patterns are formed as in (A). (F) The computed Gibbs free energy at 300K for a cylindrin forced to a fibril. The reaction coordinate (ΔRMSD) measures the difference in root-mean-squared deviation from the two end points: the cylindrin and the in-register anti-parallel beta-sheet (IAB). The cylindrin set the free energy minimum (point 1). The transition was initiated by disrupting the weak interface (points 2-3). As the cylindrin unrolls, the weak interface requires complete dissociation of backbone hydrogen bonds (points 4-5), whereas the strong interfaces maintains hydrogen bonding (point 6). The IAB has a higher free energy than the cylindrin (point 7), and when two associate and interdigitate to form a steric-zipper (point 8) the free energy drops to 5.2 kcal/mol/peptide lower than the cylindrin (Table 4).

FIG. 3 shows G6V (GDVIEV) (SEQ ID NO:2) and cylindrin peptides display amyloid biophysical characteristics. (A) Electron micrographs (EM) of negatively stained fibers formed by G6V (left side). Scale bar is shown. X-ray fibril diffraction pattern of dried G6V fibrils exhibit meridional reflections at 4.8 Å spacing and equatorial reflections at 12 Å (right side) spacing. Reflection rings are labeled. (B) Representative EM of various K11V-related peptides and their fibrils (described in methods). Cylindrin peptide abbreviations (see Table 1) and scale bar are shown. (C) X-ray powder diffraction pattern of K11V-TR fibrils, reflections are consistent with cross-beta sheet structure, as described for (A). (D) K11V-TR fibril sample was incubated with congo-red prior to drying on a cover slip (upper image). The fibrils are congo-red positive, displaying apple-green birefringence under polarized light (lower image). (E) Immuno-dot blot analysis of solutions at equal concentration based on their oligomeric state of K11V-TR and negative control K11VV4W-TR with polyclonal antibody, A11 (14). Positive control Abeta40 prefibrillar oligomers (+) and negative control Abeta40 fibrils (−) are shown.

FIG. 4 shows the crystal structure of G6V (GDVIEV) (SEQ ID NO:2), the last six residues of the cylindrin peptide segment derived from alphaB crystallin (ABC). The segments form two parallel beta-sheets. The interface between the sheets is dry, containing no water. The aspartate residues form hydrogen bonds down the fibril axis (right).

FIG. 5 shows native nanoelectrospray mass spectrometry of K11VV2L and K11V-TR oligomers. The cylindrin peptide abbreviation (see Table 1) is labeled for the respective mass spectrum in the upper right corner of the inset. (A) Mass spectrum of cylindrin peptide, K11VV2L dissolved directly in 200 mM ammonium acetate buffer. Expansion of the hexameric oligomers with n-1 and n-2 dissociation products (see for review (13)), shown in inset. The native ions 1455.91 and 1476.71 correspond to six peptide chains with a +5 charge state. The 1455.91 ion corresponds to a measured monoisotopic mass of 7270.4974 Da with a mass accuracy of 0.55 ppm. The n-1 (pentamer) and n-2 (tetramer) dissociated species are located under the respective label, corresponding to ions with +4 and +3 charge states, respectively. The 1231.76 ion corresponds to the K11VV2L peptide chain with a +1 charge state. (B) Native mass spectrum of K11V-TR purified by size exclusion chromatography (see methods for details). The oligomer of three peptide chains, 1547.75 m/z with +5 charge state, has a measured monoisotopic mass of 7729.6852 Da with a mass accuracy of 3.93 ppm (FIG. 1D). The labeled +2 and +6 charge state ions correspond to one and four peptide chains, respectively. As only hexamers were observed for the single chain peptides, we suspected the K11V-TR tetramer may arise from non-specific aggregation during ion formation (see for review (37)). Native nanoelectrospray was performed on diluted samples of K11V-TR resulting in spectra with only trimers present (data not shown), being consistent with the higher order oligomers resulting from non-specific aggregation during ionization.

FIG. 6 shows native nanoelectrospray mass spectrometry and collision induced dissociation (CID) of the K11V-TR cylindrin complex in the gas phase. (A) Ion isolation of the parent ion of 1548 of +5 charge (shown in FIG. 1D), corresponding to the oligomeric complex of three K11V-TR peptides, was subject to CID. The resulting dissociating products were a monomer and dimer corresponding to the ions of 1289 (shaded in green, zoom shown in panel B) and 1719 (shaded in purple, zoom shown in panel C), respectively. (D) Ion isolation of the +3 dimer ion of 1719 (panel A, shaded in purple) was subjected to CID. The dimer dissociated into monomeric units of +2 (shaded in orange, zoom shown in panel E) and +1 (shaded in yellow, zoom shown in panel F) charge. The K11V-TR cylindrin complex of three peptide chains in the gas phase followed charge state reduction into monomeric units, demonstrating the SEC-HPLC purified complex is composed of three peptides chains consistent with our crystal structures.

FIG. 7 shows cell toxicity of cylindrin peptides in HEK293, HeLa, PC12, and SH-SY5Y cell culture lines. (A) The cylindrin forming peptide, K11V-TR, displays concentration dependent cell toxicity in all four cell lines. The mutant cylindrin peptide, K11VV4W-TR, designed to disrupt oligomer formation, show little to no cell toxicity. The cylindrin forming peptides in oligomeric form display cell toxicity, while the non-oligomer forming peptide displays no toxicity. Bars are color-coded for different peptide concentrations as shown in the figure legend on the right with cylindrin peptide abbreviations as listed in Table 1. Each bar represents the mean and SEM of twelve replicates from three independent tests. (B) A summary table of statistical significance for comparison of K11V-TR to the non-cylindrin forming peptide K11VV4W-TR at similar peptide concentrations. Student's t-test (N=12): *, P<0.05; **, P<0.01; and ***, P<0.001.

FIG. 8 shows that cylindrin tandem repeat peptides do not induce membrane leakage. Liposome dye-release experiments were performed with K11V-TR (wild-type), K11VV2L-TR (contains V2L mutation in each repeat), or hIAPP8-37 (residues 8-37) peptides. The concentrations used in experiments are shown (inset). Peptides were incubated with calcein-containing liposomes (details provided in experimental methods), and calcein fluorescence was measured over time. The hIAPP8-37 was a positive control (15, 16), and leakage was observed up to 60%. The K11V-TR or K11VV2L-TR peptides reached a maximum leakage of 10%, despite the concentration being 10 times higher compared to the hIAPP8-37 peptide. This suggests that membrane disruption is not the main mechanism of toxicity for cylindrin.

FIG. 9 shows representative purification and purity of recombinant tandem repeat cylindrin peptides. (A) Reverse phase HPLC (RP-HPLC) chromatogram of tandem repeat cylindrin peptide, K11V-TR, post TEV protease treatment. Absorbance at 220 nm and 280 nm are shown by green and dashed red lines, respectively. The peak absorbing at 220 nm corresponding to K11V-TR is highlighted by a shaded yellow box. Peak fractions were pooled and lyophilized. (B) Lyophilized peptide was dissolved in buffer (40% Acetonitrile, 0.1% TFA) and subject to nanoelectrospray mass spectrometry. The two most abundant ions, with charge states labeled, correspond to a molecular mass consistent with the K11V-TR peptide. Under these conditions ions of a +5 and +3 charge were observed, labeled by red stars, corresponding to oligomeric molecular masses consistent with three and two K11V-TR peptides, respectively.

FIG. 10 shows schematics of unrolled anti-parallel regular beta-sheet barrels and cylindrin. Shown in each schematic is shear number, S, and the mean slope of the strands to the central axis of the barrel, in degrees, as described by Murzin et al. 1994 (38), not drawn to scale. (A) For ideal regular beta barrels of six strands with a shear number of 12 and 6, the mean slope is 56° and 37° (38), respectively. Hydrogen bonds are shown by dashed yellow lines. (B) The cylindrin with a shear number of 6 has a mean slope of ˜35°. Hydrogen bonds are shown as described in FIG. 2. The mean slope of cylindrin is similar to that for the regular S=6 mode, but the sheet-to-sheet offset and hydrogen bonding differ. The sequence shown in the figure, KVKVLGDVIEV is SEQ ID NO:3.

FIG. 11 shows the two reference structures used in the molecular dynamics simulations: (A) cylindrin and (B) cylindrin in-register anti-parallel beta sheet amyloid fibril model (only half modeled). The root mean squared deviation (RMSD) between these two structures is 8.5 Å.

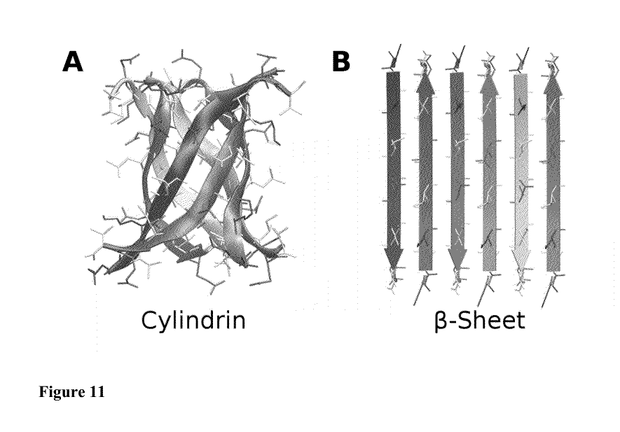

FIG. 12 shows MD simulation setup and fibril model of cylindrin, K11V. (A) The cylindrin in-register fibril model was placed in a periodic solvation box. (B) A snapshot of the bilayer after 10 ns MD simulation. The bilayer interface was dehydrated throughout the simulation period.

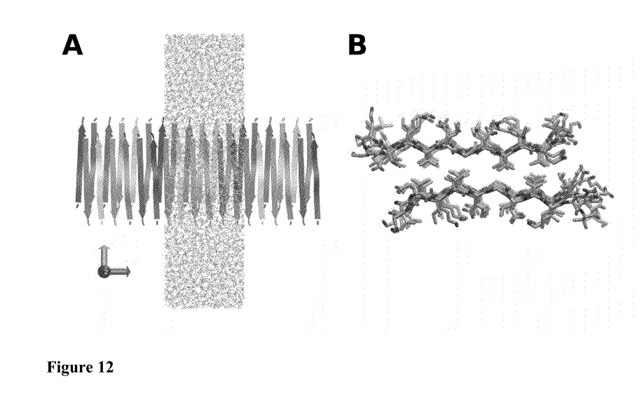

FIG. 13 shows a cylindrin model of Abeta, viewed both perpendicular to (left) and down (right) the cylindrin axis. This model, one of several possible, is built from three identical antiparallel pairs of Abeta segments: Abeta(26-40) and Abeta(28-42). In the view perpendicular to the cylindrin axis, apolar sidechains are green. The view down the cylindrin axis shows apolar sidechains filling the cylinder.

FIG. 14 shows the structure of the SOD1-derived peptide KVKVWGSIKGL (SEQ ID NO:61). a. Four peptide strands, showing how pairs of strands align out-of-register (compare green and red ‘down’ arrows) and how bonded pairs have their C-termini pointing toward one-another (red strands). b. One turn of the antiparallel beta-sheet corkscrew. Each peptide contributes one beta-strand, and 16 such strands H-bond out-of-register to form one turn of the corkscrew. c. Surface rendering of approximately two turns of the corkscrew, showing the strip of hydrophobic (red) tryptophan residues on the exterior, and the hydrophobic residues just barely visible in the interior (concave strip of orange). d. Section of the interior showing the importance of Gly6 (purple). Valines (green sticks) flank the glycine position and would overlap with a side chain if the glycine were mutated to another residue like valine.

### DESCRIPTION

This application relates, e.g., to the design, isolation and characterization of stable, artificially generated small amyloid oligomers named “cylindrins,” to methods of designing and making them, and to methods of using them to isolate putative agents which inhibit the cell toxicity (cytotoxicity) of the cylindrins.

A “cylindrin” of the invention, as used herein, is a non-covalent assembly of substantially identical chains of an amyloid or amyloid-related protein,

wherein each chain has a length of about 10-100 amino acid residues and comprises
a single copy of a cylindrin-forming segment, or
tandem adjacent copies (e.g., 1, 2, 3, 6 tandem copies) of a cylindrin-forming sequence, optionally separated by spacers, or
adjacent copies of a first cylindrin-forming segment and a second complementary segment of the first cylindrin-forming segment, optionally separated by spacers,
wherein at least about ⅔ of the amino acid residues in the chain are cylindrin-forming segments,

wherein the cylindrin is a curved beta sheet formed from anti-parallel out-of-register extended protein strands, which is substantially filled with packed side chains.

The cylindrins of the present invention, which are artificially derived, differ from naturally occurring cylindrins, at least because the cylindrins of the present invention are produced synthetically (e.g. by chemical synthesis or by expression from a synthetic or recombinant gene) rather than being naturally occurring; are less complex and more homogenous (at least ⅔ of the sequences are cylindrin-forming sequences, in repeated copies, whereas naturally occurring cylindrins contain many additional regions, which are not involved in the aggregation required for the formation of cylindrins); generally are considerably smaller (the chains having a total length of only about 10-100 amino acids, compared to the lengths of the chains of naturally occurring cylindrins, which are often significantly larger); and often are considerably more stable.

Cylindrins of the present invention also differ from the “steric zippers” which have previously been described for amyloid or amyloid-related proteins, at least because the two types of molecular assemblies have completely different structures. For example, cylindrins are cylindrical whereas steric zippers are nearly flat. Furthermore, cylindrins are not adhesive, whereas steric zippers are adhesive and form fibrils.

One aspect of the invention is a cylindrin (an artificially derived cylindrin), as defined above. In one embodiment of the invention (e.g. the ABC cylindrin structure shown herein), the curved beta sheet is a cylindrical barrel formed from antiparallel protein strands. In another embodiment of the invention (e.g., the SOD1 cylindrin structure shown herein), the curved beta sheet is an antiparallel beta-sheet corkscrew.

Another aspect of the invention is a method for making a cylindrin, comprising

identifying a cylindrin-forming segment from a amyloid or an amyloid-like protein of interest, by using the cylindrin structure of a known cylindrin as a profiled structure in a method of 3D profiling,

synthesizing copies of the cylindrin-forming segment (e.g. as individual or as tandem copies), and

allowing the copies to form oligomers (e.g., in solution),

thereby forming a cylindrin.

Not all of the preceding steps need be carried out in order to make a cylindrin. For example, in some embodiments of the invention, the sequence of the cylindrin-forming segment has already been determined, and/or a cylindrin has already been synthesized, before the copies of the cylindrin-forming segments are allowed to form oligomers in solution.

The preceding methods of making a cylindrin can further comprise (a) testing the cylindrin for properties of a cylindrin, e.g. for its ability to inhibit cylindrin-mediated cell toxicity; and/or (b) crystallizing the cylindrin and/or characterizing (determining the 3D structure of) the cylindrin by X-ray crystallography.

Other aspects of the invention include: a polynucleotide encoding a cylindrin-forming segment or tandem copies thereof; an expression vector, comprising the polynucleotide, operably linked to a regulatory control sequence (e.g., a promoter or an enhancer); a cell comprising the expression vector; and a method of making a cylindrin or segment of cylindrin, comprising cultivating the cell and harvesting the polypeptide thus generated.

Another aspect of the invention is a method for identifying (designing, selecting, screening for) a putative agent that inhibits or reduces cylindrin-mediated cell toxicity, comprising

contacting cells with a cytotoxic cylindrin and with a putative inhibitory agent, and

measuring (determining) viability of the cells which were contacted with the putative agent compared to the viability of control cells which were not contacted with the putative inhibitory agent,

wherein a putative agent that results in a statistically significantly greater viability of the cells that were contacted with the putative agent than the control cells is a candidate for an agent that inhibits cylindrin-mediated toxicity.

In some embodiments of the invention, a 3D structure of a cylindrin determined by a method of the invention (e.g., the SOD1 structure described herein) can be used as a profiled structure for identifying cylindrin-forming sequences of amyloid or amyloid-related proteins.

Another aspect of the invention is a computer-readable medium, providing the structural representation of a cylindrin of the invention, as described herein.

Another aspect of the invention is a kit for making and/or characterizing a cylindrin, or for carrying out a method of the invention, such as method for making a cylindrin or a method screening for cylindrin inhibitors.

In initial studies, the present inventors chose to work with alphaB crystallin (ABC), a protein that is a chaperone (12-14) which forms amyloid fibrils (15). This protein was selected because the fibrils form more slowly than those of, e.g., the Amyloid beta peptide (Abeta) of Alzheimer's disease or Islet Amyloid polypeptide (IAPP), so that the oligomeric state may be trapped prior to the onset of fibrillization. The inventors first identified a segment of this amyloid-forming protein which forms an oligomeric complex exhibiting properties of other amyloid oligomers: beta-sheet-rich structure, cytotoxicity, and recognition by an anti-oligomer antibody. That is, the structures satisfy the definition of a small amyloid oligomer set forth in the Background section above. The ABC cylindrin binds to a conformational antibody which also binds to Abeta oligomers, indicating that the two have similar conformations.

The X-ray-derived atomic structure of this artificially derived ABC amyloid oligomer reveals a cylindrical barrel, formed from six anti-parallel, protein strands. This ABC structure is representative of the generic class of structures which the inventors have named cylindrins. Cylindrins offer models for the hitherto elusive structures of amyloid oligomers. These cylindrins, which are small toxic protein oligomers (toxic agents), are believed to be the etiologic agents of several amyloid diseases, including Alzheimer's, Parkinson's, diabetes type 2, and the prion conditions. The peptide elements which form the chains of an oligomeric cylindrin complex are sometimes referred to herein as “cylindrin-forming segments” or “cylindrin-forming sequences” or “cylindrin-forming peptides.” Example I discusses the design, isolation and characterization of the ABC cylindrin.

The inventors subsequently identified cylindrin-forming segments (sequences) of Abeta, using the 3D structure (e.g. the molecular coordinates of the 3D structure) of the ABC cylindrin as a profiled structure in the 3D profiling method described in Bowie et al. (1991) Science 253, 164-170, and showed that the cylindrin structure for ABC is compatible with the sequence segment from the Abeta protein. These studies are presented in Example II.

In a further expansion of the method, the inventors, again using the ABC cylindrin structure as a profiled structure, identified cylindrin-forming segments from superoxide dismutase I (SODI), a protein which has been implicated in Amyotrophic lateral sclerosis. The cylindrins formed from these segments exhibit a structure similar to, but somewhat different from, the ABC cylindrin 3D structure. The SOD1 cylindrin structure is an antiparallel beta-sheet corkscrew. These studies are presented in Example III.

Using comparable techniques, based, e.g., on the 3D structure described herein of the ABC cylindrin, one of skill in the art can readily identify cylindrin-forming sequences for a variety of other amyloid or amyloid-related proteins. For example, Example IV shows cylindrin-forming sequences determined for the additional, representative, amyloid proteins IAPP, prion protein (PrP), α-synuclein, Tau, and TDP43. Using conventional techniques, including some described herein, a skilled worker can readily synthesize (or express) such peptides, generate cylindrins from them; confirm that they exhibit toxic properties; and use them to identify putative agents which inhibit cylindrin-mediated functions, such as cell toxicity. Furthermore, 3D structures determined as described herein can be used as profiled structures for identifying cylindrin-forming sequences from additional amyloid or amyloid-like proteins.

A cylindrin comprises substantially identical chains of an amyloid or an amyloid-related protein. A cylindrin, as used herein, comprises a cylindrin-forming segment of an amyloid or of an amyloid-related protein. In some places herein, the terms “amyloid” and “amyloid-related” are used interchangeably. It is to be understood that a cylindrin from an amyloid-related protein has similar properties to a cylindrin from an amyloid protein.

An “amyloid-related protein,” as used herein, refers a polypeptide having the common properties of an amyloid protein, but not yet officially recognized by the Nomenclature Committee of the International Society of Amyloidosis, who define amyloid diseases and proteins. An “amyloid protein,” as used herein, is one of a class of proteins having common properties, including, e.g., the ability to polymerize to form a cross-beta structure, in vivo, or in vitro. Many of these amyloid and amyloid-related proteins exhibit classic histopathological characteristics such as Congo red birefringence. Inappropriately folded (misfolded) versions of the proteins interact with one another or other cell components to form insoluble fibrils (e.g. plaques or tangles). A skilled worker will recognize a wide variety of amyloid or amyloid-related proteins that can be used to derive cylindrins by a method as described herein and/or to carry out a method of the invention, such as a method to select inhibitors of cylindrins. These proteins have been implicated in the etiology of a variety of diseases or conditions, including neurodegenerative ones, and include, e.g., beta amyloid (Alzheimer's disease, cerebral amyloid angiopathy), tau (Alzheimer's disease and a large number of tauopathies, including frontotemporal dementia and progressive supranuclear palsy), amylin (diabetes type 2), Prion protein (PrP-Creutzfeldt-Jacob Disease, fatal familial insomnia, other prior-based conditions), Superoxide dismutase1 (SOD1-ALS), TAR DNA-binding protein-43 (TDP-43-ALS), RNA-binding protein FUS (Fused in Sarcoma (FUS-ALS), alpha-synuclein (Parkinson's disease), p53 (many cancers), transthyretin (several different amyloidosis conditions), beta 2 microglobulin (dialysis related amyloidosis), insulin (injection amyloidosis) and lysozyme (lysozyme amyloidosis). For additional amyloid and amyloid-related proteins from which a skilled worker can derive cylindrins according to a method of the invention, and diseases or conditions mediated by them, see Sipe et al. (2012) Amyloid 19(4), 167-170, which is incorporated by reference herein. It is to be understood that the discussion herein with regard to amyloid-related proteins, e.g., in the context of cylindrins derived from them, is also applicable to amyloid proteins, and vice versa. Such conditions are sometimes referred to herein as “amyloid-mediated” or “cylindrin-mediated” conditions or diseases. A disease or condition that is “mediated” by, or “associated with” an amyloid or cylindrin is one in which the amyloid or cylindrin plays a biological role. The role may be direct or indirect, and may be necessary and/or sufficient for the manifestation of the symptoms of the disease or condition. It need not necessarily be the proximal cause of the disease or condition.

“Fibrillation” or “fibrillization” refers to the aggregation of amyloid molecules to form fibers.

A “cylindrin-forming segment” (sometimes referred to herein as a cylindrin-forming peptide or cylindrin-forming sequence) of an amyloid or amyloid-related protein is a segment of about 7-15 amino acids of the amyloid or amyloid-related protein which self-aggregates or aggregates with a complementary sequence to form a cylindrin structure, in vitro or in vivo.

“Complementary,” as used herein, is defined as follows: A cylindrin formed from two distinct segments, a first segment and a second segment, which are complementary to each other, will have an alternating pattern of these segments, and the side chains of these segments will pack to mostly fill the internal space of the cylindrin. Thus, the second, complementary sequence is one of similar length as the first segment, whose side chains can pack with those of the first sequence to substantially fill the internal space of the cylindrin.

Each “chain” of an amyloid or amyloid-related protein in a cylindrin has a length of about 10-100 amino acid residues. In embodiments of the invention, these peptide chains contain, for example, a single copy of a cylindrin-forming segment; or tandem adjacent copies of a cylindrin-forming sequence; or adjacent copies of a first cylindrin-forming segment and a second complementary segment of the first cylindrin-forming segment. In embodiments of the invention, tandem sequences are separated by suitable spacer sequences.

In embodiments of the invention, the chain comprises one copy, or 2, 3, 4, 5, 6 or more tandem copies, of a cylindrin-forming segment of the invention. For example, in the case of an 11 amino acid cylindrin-forming segment in a cylindrin having the shape of a cylindrical barrel, the cylindrin can contain 6 chains of the 11 amino acid cylindrin-forming segment; 3 chains of an about 25 amino acid peptide comprising two adjacent tandem copies of the 11 amino acid cylindrin-forming segment, optionally separated by suitable spacers, or one copy of the segment adjacent to a complementary copy of the segment, optionally separated by suitable spacers; 2 chains of an about 45 amino acid peptide consisting of a three adjacent tandem copies of the 11 amino acid cylindrin-forming segment, optionally separated by spacers, or alternating complementary copies, optionally separated by suitable spacers; or 1 chain containing six adjacent tandem copies of the 11 amino acid cylindrin-forming segment, or alternating complementary copies, optionally separated by suitable spacers. Some such structures are described herein. The chains are arranged in an anti-parallel fashion, so the chains containing two copies of a segment, for example, contain one segment in one orientation and a second segment in the opposite (complementary) orientation; when the two segments fold to form a hairpin-like structure, the two segments line up in an antiparallel fashion.

In some embodiments of the invention, in which more than one (e.g., two) different cylindrin-forming segments are identified in an amyloid or amyloid-related protein, a chain can comprise both of these segments, for example arranged in tandem. See, e.g., some of the SOD1 peptides shown in Table 7. In artificial cylindrins comprising, e.g., two different cylindrin-forming segments, the same sorts of arrangements of segments can be formed as described above. In addition, a chain can have mixtures of the two types of segments, e.g., two copies of one segment and four copies of the other.

Suitable spacers will be evident to a skilled worker who is familiar with structural biology. One function of the spacers is to allow portions of the chains (e.g., a segment and a complementary segment) to fold back upon one another to allow the formation of antiparallel strands in a cylindrin. When a tight turn is desired, such as for the formation of a cylindrical barrel, amino acids which allow for great flexibility can be used. These include, e.g., various combinations of at least two amino acids selected from, e.g., glycine, asparagine and proline. Typical spacers include Gly-Gly, Gly-Pro and Asn-Gly. When looser turns are possible, such as in the corkscrew structures formed by SOD1 cylindrins, larger “loops” of as many as about 20 amino acids intervening between cylindrin-forming segments can be tolerated. See, e.g., some of the SOD1 peptides in Table 7. The upper case letters represent the cylindrin-forming segments which are involved in the formation of the cylindrin, whereas the lower case letters represent spacers, including such loops.

The chains in a cylindrin are “substantially” identical. By “substantially identical” is meant that the chains contain identical cylindrin-forming segments, but may differ in other respects. For example, when tandem copies or complementary segments of cylindrin-forming segments are present, amino acid spacers between them may differ in the chains forming the cylindrin. In general, such variation will not significantly affect the structure of the cylindrin. Spacers, including variant spacers, are selected so that properties of the cylindrins, such as their cytotoxicity, are not negatively impacted. The chains of a cylindrin “consist essentially of” the cylindrin-forming segments. The additional sequences, such as intervening sequences and/or spacers, do not materially affect the basic and novel characteristic(s) of the cylindrin, such as its cytotoxicity.

In a cylindrin of the invention, at least about ⅔ of the amino acid residues in each chain are cylindrin-forming segments, which can be repeated in a regular, symmetric, fashion. This is one of the features which distinguishes the artificially generated cylindrins of the invention from naturally occurring cylindrins. In naturally occurring cylindrins, the cylindrin-forming segments are buried within a longer protein sequence, including many regions which are not related to the formation of a cylindrin; so the naturally occurring cylindrins are far more complex than the artificially generated cylindrins of the invention.

Some of the cylindrins of the invention, such as the ABC cylindrin described herein, contain six chains of a substantially identical cylindrin-forming sequence. In other embodiments of the invention, such as the beta sheet corkscrew of the SOD1 cylindrin, the number of chains is potentially infinite. Other cylindrins of the invention can have intermediate numbers of chains.

A cylindrin is formed from anti-parallel out-of-register extended protein strands. By “extended” is meant that the peptide backbone is nearly flat, e.g. that the cylindrin-forming segments adopt a beta-strand structure in which the backbone torsion angles approach ±180°. (In an “ideal” antiparallel beta-strand, the phi angle is roughly −140°, and the psi angle is roughly +140°, but in real beta-strands, such as those in a cylindrin, these values can vary by)±40°.

A cylindrin is a “curved” beta sheet. By “curved” is meant that the sheet is closed or partially closed on itself, such that amino acid side chains from one face of the sheet are at least partially buried and shielded from the solvent.

A cylindrin of the invention is “substantially” filled with packed side chains. As used herein, this term means that water molecules do not occupy more than about 15% of the volume of the interior of the cylindrin.

In many cylindrins of the invention, there is an important glycine (Gly) residue which occupies a central location in the cylindrin-forming segment and points toward the interior of the cylindrin (the interior of the curvature). The importance of the Gly residue being in this position is that glycine's lack of a side chain provides space for other side chains to pack without overlapping. A residue other than glycine would take up too much space, forcing other side chains apart, and reducing or eliminating the curvature of the beta-sheet (disrupting the cylindrin) in the center of the barrel or corkscrew, which is important for the toxicity of the cylindrins. Support for the importance of the glycine residue is provided by the mutagenesis studies of ABC discussed herein, and by the studies of SOD1 in patients with ALS, which show that although a wide variety of mutations are observed among the patient population, the glycine residue at this position is invariantly present.

In one embodiment of the invention, a cylindrin-forming segment, chain or cylindrin is isolated or purified, using conventional techniques such as the methods described herein. By “isolated” is meant separated from components with which it is normally associated, e.g., components present after the cylindrin is synthesized. A “purified” cylindrin can be, e.g., greater than 90%, 95%, 98% or 99% pure.

In embodiments of the invention, the cylindrin is detectably labeled. Labeled cylindrins can be used, e.g., to better understand the mechanism of action and/or the cellular location of cylindrins. Suitable labels which enable detection (e.g., provide a detectable signal, or can be detected) are conventional and well-known to those of skill in the art. Suitable detectable labels include, e.g., radioactive active agents, fluorescent labels, and the like. Methods for attaching such labels to a protein, or assays for detecting their presence and/or amount, are conventional and well-known.

A method of the invention can comprise using the molecular structure of a known cylindrin (e.g. the ABC cylindrin or the SOD1 cylindrin described herein) as a profiled structure in a method of 3D profiling, in order to identify a cylindrin-forming segment from another amyloid or amyloid-related protein of interest.

A skilled worker, after having become aware of the cylindrin structures and methods described herein, can identify cylindrin-forming segments of any amyloid or amyloid-related protein of interest, using, e.g., the 3D profiling method described in the paper of Bowie et al (1991) “A method to Identify Protein Sequences That Fold into a Known Three-Dimensional Structure” Science 253, 164-170. A computer program to carry out this procedure is available from the inventors' laboratory. Other methods for identifying cylindrin-forming segments from scratch might be molecular modeling and calculating energies, or running molecular dynamics simulations, but the method of Bowie et al is preferred.

Briefly: If a given 3D structure (e.g. the atomic coordinates) is known, one can use a 3D profile method to find amino acid sequences which are compatible with that 3D structure. The 3D structure is referred to herein as a “profiled structure.” That is, these sequences, which may be segments of full proteins, can fold into the given profiled structure. In the profiling method of Bowie et al., amino acid sequences are identified which are most compatible with the environments of the residues in the 3D structure. These environments can be described by: (i) the area of the residue buried in the protein and inaccessible to solvent; (ii) the fraction of side-chain area that is covered by polar atoms (0 and N); and (iii) the local secondary structure.

For steric zippers, the procedure of Thompson et al. (2006) Proc Natl Acad Sci USA 103, 4074-4078 was one application of 3D profiling. In that case, the profiled structure was the first steric zipper structure described, which consists of two sheets, each a stack of 6-residue segments which are self-complementary. Using this structure as the profile, other six residue segments were found that were predicted to form the same sort of steric-zipper structure, but that have different amino acid sequence.

For cylindrins, the same procedure is used, but now the profiled structure is a cylindrin structure described herein (such as a cylindrin from alpha B crystalline). This profiling procedure can be used to identify cylindrin-forming sequences from amyloid proteins, such as, e.g., Abeta, tau, SOD1, alpha-synuclein, and IAPP. Example II shows the application of this method for AP; Example III shows its application to SOD1; and Example IV shows its application to a variety of other amyloid proteins. Many other cylindrin-forming segments can be identified from other amyloid proteins by one of skill in the art, using comparable procedures.

In the representative method described in Example III, the profiled structure is the ABC cylindrin 3D structure. This can be found, for example, at the world wide web site rcsb.org/pdb/files/3SGO.pdb.; the atomic coordinates are provided in Table 5. In another embodiment of the invention, the profiled structure is the SOD1 cylindrin structure that is determined herein. This can be found, for example, at the world wide web site kv11_corkscrew_new_asu.pdb; the atomic coordinates are provided in Table 6.

Once the sequence of a cylindrin-forming segment has been determined, a peptide comprising that sequence, or multiple copies of the peptide as described elsewhere herein, can be synthesized (e.g., chemically or by recombinant expression in a suitable host cell) by any of a variety of art-recognized methods. In order to generate sufficient quantities of cylindrins for use in a method of the invention, such as for use in a cell toxicity assay, a practitioner can, for example, using conventional techniques, generate nucleic acid (e.g., DNA) encoding the peptide and insert it into an expression vector, in which the sequence is under the control of an expression control sequence such as a promoter or an enhancer, which can then direct the synthesis of the peptide. For example, one can (a) synthesize the DNA de novo, with suitable linkers at the ends to clone it into the vector; (b) clone the entire DNA sequence into the vector; or (c) starting with overlapping oligonucleotides, join them by conventional PCR-based gene synthesis methods and insert the resulting DNA into the vector. Suitable expression vectors (e.g., plasmid vectors, viral, including phage, vectors, artificial vectors, yeast vectors, eukaryiotic vectors, etc.) will be evident to skilled workers, as will methods for making the vectors, inserting sequences of interest, expressing the proteins encoded by the nucleic acid, and isolating or purifying the expressed proteins.

Peptides synthesized as above (e.g. individual, single copy cylindrin-forming segments, or chains comprising one or more cylindrin-forming segments) can be purified by conventional techniques such as the exemplary ones described herein. Generally, the peptides are lyophilized before storage.

In order to form cylindrins from the peptides, in some embodiments of the invention the peptides are reconstituted by dissolving the lyophilized peptide in water or buffer and are allowed to form oligomers in solution, under conditions in which the aggregates spontaneously form. The conditions for forming particular cylindrins can vary, depending on the cylindrin. Suitable conditions can be determined readily by a skilled worker, using empirical procedures. In some embodiments, the peptides are incubated under close to physiological conditions (e.g., a temperature between about 20-37° C., about neutral pH, mM or μM monovalent and/or divalent salts). In other embodiments, more “extreme” conditions are required (e.g., a temperature as low as 4° C. or as high as 65° C. or more; pH as low as about 2 or as high as about 11). Depending on the cylindrin and the conditions employed, a cylindrin may form within a matter of minutes, or it may take a considerably longer period of time, e.g., days, weeks or months. Some typical conditions for forming cylindrins are shown in the Examples.

Cylindrins of the invention can be tested for cell toxicity, using conventional methods that are well-known to those of skill in the art. Assays include those for measuring a variety of different markers that indicate the number of dead cells (cyototoxicity assay), the number of live cells (viability assay), the total number of cells, or the mechanism of cell death (e.g. apoptosis, necrosis, membrane leakage, etc.) In one embodiment of the invention, cylindrin-mediated cell toxicity is monitored by assaying for cell viability. For example, protease biomarkers have been identified that allow researchers to measure relative numbers of live and dead cells within the same cell population. The live-cell protease is only active in cells that have a healthy cell membrane, and loses activity once the cell is compromised and the protease is exposed to the external environment. The dead-cell protease cannot cross the cell membrane, and can only be measured in culture media after cells have lost their membrane integrity (Niles et al. (2007) Anal. Biochem. 366, 197-206). Cytotoxicity can also be monitored using the 3-(4,5-Dimethyl-2-thiazolyl)-2,5-diphenyl-2H-tetrazolium bromide (MTT) or MTS assay. This assay measures the reducing potential of the cell using a colorimetric reaction. Viable cells will reduce the MTS reagent to a colored formazan product. This assay is described in the Examples herein. A similar redox-based assay has also been developed using the fluorescent dye, resazurin. In addition to using dyes to indicate the redox potential of cells in order to monitor their viability, researchers have developed assays that use ATP content as a marker of viability (Riss et al. (2004) Assay Drug Dev Technol 2, 51-62). Such ATP-based assays include bioluminescent assays in which ATP is the limiting reagent for the luciferase reaction (Fan et al. (2007) Assay Drug Dev Technol 5, 127-36). Cytotoxicity can also be measured by the sulforhodamine B (SRB) assay, WST assay and clonogenic assay. A label-free approach to follow the cytotoxic response of adherent animal cells in real-time is based on electric impedance measurements when the cells are grown on gold-film electrodes. This technology is referred to as electric cell-substrate impedance sensing (ECIS). Label-free real-time techniques provide the kinetics of the cytotoxic response rather than just a snapshot like many colorimetric endpoint assays. Other suitable assays will be evident to those of skill in the art.

The cells used in assays for cylindrin toxicity can be any of a variety of cell types which will be evident to a skilled worker. For example the cells can be eukaryotic, vertebrate, mammalian, such as the four mammalian cell lines discussed in the Examples (HeLa, HEK293, PC-12, and SH SY5Y), or other types of cells that will be evident to a skilled worker. In one embodiment, the cell type which is used is appropriate for the particular cylindrin being tested. For example, pancreatic cells or cell lines can be used to test for toxicity of the IAPP cylindrin. In some embodiments of the invention, neuronal cell lines are used. In other embodiments, motor neurons are generated by differentiating iPS cells (or other stem cells) with suitable agents.

In one embodiment of the invention, expression vectors in which cylindrins are expressed are introduced (e.g., with a phage or other viral vector) into cells, such as motor neurons; cylindrins are allowed to form in vivo, and toxicity of the cylindrins is assessed. These cells can also be used to assay for putative agents that inhibit cytotoxicity of cylindrins.

In some embodiments, cells are proliferating when they are contacted with the cylindrin.

In some embodiments, cells are cultured in a suitable culture medium, e.g. as is described in Example I, and after a suitable period of time, a cylindrin which has been allowed to form in solution is added to the culture medium. In other embodiments, a peptide or chain comprising one or more copies of a cylindrin-forming segment is added directly to the culture medium and is allowed to form a cylindrin in the medium or after it has entered a cell.

Cylindrins which have been shown to be cytotoxic can be used in screening assays to design and/or select (screen for) putative agents (drugs) which inhibit or reduce their toxic effects. These agents are sometimes referred to herein as “cylindrin-inhibitors” or “cylindrin-inhibitory agents.”

One aspect of the invention is a method for identifying (designing, selecting, and/or screening for) a putative agent that inhibits or reduces cylindrin-mediated cell toxicity, comprising contacting a cell with both a cylindrin of interest and a putative inhibitory agent, and determining if the agent inhibits or reduces the cytotoxicity brought about by the cylindrin to a statistically significant degree compared to the cytotoxicity when the putative agent is not contacted with the cell.

As used herein, the singular forms “a,” “an,” and “the” include plural referents unless the context clearly dictates otherwise. Thus, for example, “a” cell, as used above, can be two or more cells.

The cell can be contacted with the putative inhibitory agent concomitantly, after, or before it is contacted with the cylindrin. The timing and relative order of the addition of the cylindrin and the putative agent, and of the measurement of cytotoxicity, can be optimized empirically, following conventional procedures.

Suitable controls will be evident to a skilled worker. For example, cells can be cultivated in parallel with the treated cells, but not contacted with a cylindrin, to determine a base line for cell viability in the absence of a toxic cylindrin. Furthermore, instead of being contacted with a putative cylindrin inhibitor (a test substance), cells which are contacted with a cylindrin can also be contacted with a control substance, such as water, buffer, or cell culture medium which is known not to inhibit cylindrin-mediated toxicity, or they cannot be contacted (treated) at all.

Cytotoxicity can be measured by any of a variety of conventional assays, including those discussed above. In embodiments of the invention, the method comprises measuring (determining) the viability of cells which were contacted with the putative agent compared to the viability of control cells which were not contacted with the putative inhibitory agent.

Assays other than toxicity assays can also be used to determine if a putative agent inhibits a function of a cylindrin. These assays can measure, e.g., the ability of a putative agent to bind specifically (preferentially compared to a control) to a cylindrin; or the ability of a putative agent to alter the distribution of oligomer sizes in a cell. In other embodiments, the assay can measure the ability of a putative agent to bind specifically to a nucleic acid encoding a cylindrin segment or chain; to inhibit the synthesis of a cylindrin peptide or chain, or a nucleic acid encoding it; to inhibit or enhance aggregation of cylindrin-forming segments into a cylindrin, directly or indirectly; to cleave or otherwise inactivate the protein or nucleic acid; or to otherwise interfere with (inhibit) an activity that is responsible for, or contributes to, symptoms or other manifestations of the amyloid disease or condition.

A putative agent which results in a statistically significant amount of inhibition of one or more functions of a cylindrin (e.g. the inhibition or reduction cylindrin-mediated or induced cellular toxicity, leading to greater viability; the specific binding to a cylindrin molecule, etc.) compared to a suitable control which lacks such inhibitory activity, is a candidate for an agent that inhibits cylindrin-mediated toxicity. Conventional methods for statistical analysis can be used.

Any of a variety of types of putative agents can be tested in a method of the invention. Because many amyloid or amyloid-related conditions are neurodegenerative conditions or diseases, it is desirable that the agents can cross the blood-brain barrier. Small molecules are particularly suitable in this respect. The term “small molecule” refers to a low molecular weight organic compound, e.g. having a molecular weight of less than about 800 Daltons (e.g. <700, 600, 500, 400, 300 Daltons). As used throughout this application, “about” means plus or minus 5% of a value.

The test compounds may be known compounds or based on known compounds. Suitable libraries of small molecule compounds will be evident to a skilled worker. These include, for example, the ZINCPharmer (world wide web site zincpharmer.csb.pitt.edu) library, which is an online interface for searching for purchasable compounds; or the following libraries: BioMol (world wide web site mssr.ucla.edu/biomol.html), Chem Div (chemdiv.com), SPECS (specs.net), Chembridge (chembridge.com) or combinatorial libraries from ChemRx (combi.chemlab.com).

Other agents that can be tested include peptides, such as circular peptides, conformational antibodies, etc.

The invention also includes computer-related embodiments, such as a computer-readable medium, providing the structural representation of a cylindrin of the invention, or for storing and/or evaluating the assay results s described herein.

Another aspect of the invention is a kit for carrying out any of the methods described herein (e.g., methods for identifying cylindrin-forming peptides, for screening for compounds which inhibit cylindrin-mediated cellular toxicity, etc).

The storage medium (computer readable medium) in which the cylindrin structural representation is provided may be, e.g., random-access memory (RAM), read-only memory (ROM e.g. CDROM), a diskette, magnetic storage media, hybrids of these categories, etc. The storage medium may be local to the computer, or may be remote (e.g. a networked storage medium, including the internet). The present invention also provides methods of producing computer readable databases containing coordinates of 3-D cylindrin structures of the invention; computer readable media embedded with or containing information regarding the 3-D structure of a cylindrin of the invention; a computer programmed to carry out a method of the invention (e.g. for characterizing the structure of a cylindrin, or for designing and/or selecting small molecule cylindrin binders or inhibitors), and data carriers having a program saved thereon for carrying out a method as described herein.

Any suitable computer can be used in the present invention.

Another aspect of the invention is a kit for carrying out any one of the methods described herein (e.g., methods for identifying cylindrin-forming segments, for screening for compounds that inhibit cylindrin-mediated cellular toxicity, etc.)

The kit may comprise a suitable amount of a cylindrin of the invention; reagents for generating a cylindrin (e.g. oligonucleotides, primers, vectors, cells etc.); reagents for assays to measure cylindrin-mediated functions or activities, such as cylindrin cytotoxicity, or to screen for agents that inhibit or reduce such activities; or the like. Kits of the invention may comprise instructions for performing a method, such as a method for screening for inhibitors. Other optional elements of a kit of the invention include suitable buffers, media components, or the like; a computer or computer-readable medium for providing profiled structures for identifying cylindrn-forming segments, or for storing and/or evaluating the assay results; containers; or packaging materials. Reagents for performing suitable controls may also be included. The reagents of the kit may be in containers in which the reagents are stable, e.g., in lyophilized form or stabilized liquids. The reagents may also be in single use form, e.g., in single reaction form for screening assays.

In the foregoing and in the following examples, all temperatures are set forth in uncorrected degrees Celsius; and, unless otherwise indicated, all parts and percentages are by weight.

### EXAMPLES

### Example I

### Cylindrins as Etiologic Agents of Amyloid Diseases

### A. Materials and Methods

### Peptide Crystallization

Synthetic peptides were purchased from CS BIO (Menlo Park, Calif.). All peptides were filtered through a 0.22 μm Ultrafree-MC centrifugal filter device (AMICON, Bedford, Mass., USA) prior to crystallization in hanging drop plates. All crystallization was performed at room temperature. KVKVLGDVIEV (SEQ ID NO:3) (K11V, see Table 1) was dissolved in water to a final concentration of 10 mM and mixed with 5 mM OrangeG (Product No. 861286, Sigma-Aldrich, St. Louis, Mo.), for a final concentration of 4 mM K11V and 3 mM OrangeG. This peptide mixture was crystallized in 0.1 M BIS-TRIS (pH 6.5), 45% 2-methyl-2,4-pentanediol (MPD), 0.2 M ammonium acetate (Index #51, Hampton Research, Aliso Viejo, Calif.). K11V-Br2, (2-Bromoallyl)-glycine substitution at position 2, was dissolved in water at 15 mg/mL, crystallized in 0.1 M TRIS (pH 7.0), 35% MPD, 0.2 M sodium chloride (Wizard #24, Emerald BioSystems, Bainbridge Island, Wash.) and crystals appeared in 1-3 days. K11V-Br8, (2-bromoallyl)-glycine substitution at position 8, was dissolved in water at 15 mg/mL and crystallized in 0.1 M HEPES (pH 7.5), 30% MPD, 0.2 M sodium citratre tribasic dihydrate (Crystal Screen #5, Hampton Research, Aliso Viejo, Calif.). KLKVLGDVIEV (SEQ ID NO:3) (K11VV2L) was dissolved in water at 10-15 mg/mL and crystallized in 0.1M TRIS (pH 7.0), 35% MPD, 0.2M sodium chloride (Wizard #24, Emerald BioSystems, Bainbridge Island, Wash.). GDVIEV (G6V) was dissolved in water at 6 mg/mL and crystallized in 2.1 M DL-Malic acid pH 7.0 (JCSG+#68, Qiagen, Valencia, Calif.).

### Recombinant Beta Cylindrin Tandem Repeat Peptide Plasmid Construction

A tandem repeat beta cylindrin peptide, K11V-TR, synthetic gene, codon optimized for E. coli, was designed using DNAWorks (1) and constructed using PCR-based gene synthesis as described (1). The synthetic gene was PCR amplified with Platinum Pfx polymerase (Invitrogen, Carlsbad, Calif.) with the N-terminal primer containing a Sad restriction and TEV protease site, and a C-terminal primer containing a stop codon and XhoI restriction site. Agarose gel purified PCR product, K11V-TR, was extracted using the QIAquick Gel Extraction Kit (Qiagen, Valencia, Calif.). Gel purified PCR product and custom vector, p15-MBP (described below), were digested with Sad and XhoI according to manufacturer's protocol (New England Biolabs, Ipswich, Mass.). The p15-MBP custom vector is a chimera constructed from the NdeI and XhoI digestion products pET15b (Novagen, Gibbstown, N.J.), and the maltose binding protein (MBP) gene from pMAL-C2X (New England Biolabs, Ipswich, Mass.), resulting in an N-terminal His-tag MBP fusion vector. Digested vector products were gel purified and extracted (as described above). DNA concentrations were determined using BioPhotometer UV/VIS Photometer (Eppendorf, Westbury, N.Y.). A ligation mixture was performed using a Quick Ligation kit (New England Biolabs, Ipswich, Mass.) according to manufacturer protocol and transformed into E. coli cell line TOP10 (Invitrogen, Carlsbad, Calif.). Several colonies were grown overnight, and plasmid containing the synthetic K11V-TR gene were purified using QIAprep Spin Miniprep Kit (Qiagen, Valencia, Calif.). The final construct p15-MBP-K11V-TR was sequenced prior to transformation into E. coli expression cell line BL21 (DE3) gold cells (Agilent Technologies, Santa Clara, Calif.).

### Recombinant Beta Cylindrin Peptide Mutant Constructs

All mutations in the DNA sequence were performed on the p15-MBP-K11V-TR plasmid using a Site-Directed Mutagenesis kit (QuickChange XL, Stratagene, La Jolla, Calif.) with site-directed primers designed using manufacturers QuickChange Primer Design Program available on-line (Stratagene, La Jolla, Calif.) according to manufacturer's protocol. The K11VV2L construct was achieved by mutation of the first glycine residue coding sequence in the linker region to a stop codon. The K11VV4W-TR was achieved by two-rounds of site-directed mutagenesis. The final constructs were sequenced prior to transformation into E. coli expression cell line BL21 (DE3) gold cells (Agilent Technologies, Santa Clara, Calif.).

### Recombinant Beta Cylindrin Peptide Expression

A single colony was inoculated into 50 mL LB Miller broth (Fisher Scientific, Pittsburgh, Pa.) supplemented with 100 μg/mL ampicillin (Fisher Scientific, Pittsburgh, Pa.) and grown overnight at 37° C. One liter of LB Miller supplemented with 100 μg/mL ampicillin in 2 L shaker flasks was inoculated with 7 mL of overnight culture and grown at 37° C. until the culture reached an OD600˜0.6-0.8 using a BioPhotometer UV/VIS Photometer (Eppendorf, Westbury, N.Y.). IPTG (Isopropyl β-D-1-thiogalactopyranoside) was added to a final concentration of 0.5 mM, and grown for 3-4 hours at 34° C. Cells were harvested by centrifugation at 5,000×g for 10 minutes at 4° C. The cell pellet was frozen and stored at −80° C.

### Recombinant Beta Cylindrin Peptide Purification

The cell pellet was thawed on ice and re-suspended in buffer A (50 mM sodium phosphate, 0.3 M sodium chloride, 20 mM imidazole, pH 8.0) supplemented with Halt Protease Inhibitor Cocktail (Thermo Scientific, Rockford, Ill.) at 50 mL per 2 L of culture volume. The re-suspended culture was incubated on ice for 15 minutes prior to sonication. Crude cell lysate was clarified by centrifugation at 14,000×g for 25 minutes at 4° C. The clarified cell lysate was filtered through a 0.45 μm syringe filtration device (HPF Millex-HV, Millipore, Billerica, Mass.) before loading onto a 5 mL HisTrap-HP column (GE Healthcare, Piscataway, N.J.). The HisTrap-HP column was washed with five column volumes of buffer A and protein eluted with linear gradient to 100% in four column volumes of buffer B (50 mM sodium phosphate, 0.3 M sodium chloride, 500 mM imidazole, pH 8.0). Protein eluted around 50-70% buffer B and peak fractions pooled. A final concentration of 5 mM beta-mercaptoethanol (BME) and 1 mM ethylenediaminetetraacetic acid (EDTA) was added to the pooled sample prior to transferring to a Slide-A-Lyzer 10,000 MWCO dialysis cassette (Pierce, Thermo Fisher Scientific, Rockford, Ill.), and dialyzed against buffer C (25 mM sodium phosphate pH 8.0, 20 mM imidazole, 200 mM sodium chloride) at room temperature overnight. The dialyzed sample was pooled and 1/500 volume of TEV protease stock (2) was added. The TEV protease reaction was incubated overnight at room temperature before loading over a 5 mL HisTrap-HP column equilibrated in buffer A. The flow through was collected, containing the recombinant beta cylindrin peptide with an additional N-terminal glycine residue resulting from TEV protease cleavage. Pooled recombinant beta cylindrin peptide was 0.22 μm filtered (Steriflip, Millipore, Billerica, Mass.) and further purified by reverse phase high performance liquid chromatography (RP-HPLC) on a 2.2×25 cm Vydac 214TP101522 column equilibrated in buffer RA (0.1% trifluroacetic acid (TFA)/water) and eluted over a linear gradient from 0% to 100% buffer RB (Acetonitrile/0.1% TFA) in 40 minutes at a flow rate of 9 mL/min. Absorbance at 220 nm and 280 nm were recorded using a Waters 2487 dual A absorbance detector (Waters, Milford, Mass.). Peak fractions containing peptide were assessed for purity by either a MALDI-TOF mass spectrometry (Voyager-DE-STR, Applied Biosystems, Carlsbad, Calif.) or direct infusion nanoelectrospray mass spectrometry using a hybrid linear ion-trap/FT-ICR mass spectrometer (7T, LTQ FT Ultra, Thermo Scientific, Bremen, Germany). Pooled fractions were frozen in liquid nitrogen and lyophilized. Dried peptide powders were stored in desiccant jars at −20° C.

### Size Exclusion Chromatography HPLC (SEC-HPLC)

One to five milligrams of lyophilized peptide was dissolved in 1 mL of water and filtered through a 0.22 or 0.45 μm Centrex MF filter (Whatman, Florham Park, N.J.). Filtered samples were injected on a 21.5 mm×60 cm Tosohaas G3000SW column (Tosoh Bioscience, King of Prussia, Pa.) equilibrated in SEC buffer (25 mM sodium phosphate, 100 mM sodium sulfate pH 6.5) at a flow rate of 3 mL/min. Absorbance at 220 nm and 280 nm were recorded using a Waters 2487 dual λ absorbance detector (Waters, Milford, Mass.). Protein standards were monitored by absorbance at 280 nm, and cylindrin peptides monitored by absorbance at 220 nm. For native nanoelectrospray mass spectrometry experiments the SEC buffer was changed to 200 mM ammonium acetate, pH adjusted to 6.5 with acetic acid.

### Recombinant K11V-TR Beta Cylindrin Peptide Crystallization

Crystals of K11V-TR were grown in hanging drop VDX plates (Hampton Research, Aliso, Viejo, Calif.) from either (i) purified oligomeric complexes or (ii) freshly dissolved peptide preparations. i) Peak fractions from SEC-HPLC in SEC buffer containing the oligomeric K11V-TR complex was concentrated using a 3,500 MWCO concentrator (Millipore, Billerica, Mass.) at 4° C. The concentrated K11V-TR buffer was exchanged by several washes in buffer (100 mM sodium chloride, 20 mM HEPES pH 7.5) followed by concentration. The buffer exchanged K11V-TR complex was concentrated to a concentration of ˜2.5 mg/mL, as judged by the Bradford assay (Bio-Rad, Hercules, Calif.) using known solutions of K11V-TR for a standard curve. ii) A microfuge tube containing a pre-weighed quantity of K11V-TR, usually a few milligrams, was chilled on ice. A given volume of ice cold water was gently added to yield a final peptide concentration of 2.5 mg/mL, and stored on ice until dissolution of peptide was complete without disturbance. Both preparations of the K11V-TR complex were either used immediately or stored at 4° C. prior to use. Crystals of K11V-TR were grown using ice cold components of a K11V-TR preparation with crystallization solution 30% MPD, 0.2M magnesium acetate, 0.1M sodium cacodylate pH 6.5 (Crystal Screen #21, Hampton Research, Aliso Viejo, Calif.). Crystallization was carried out at 10° C. Crystals from either starting preparations displayed similar X-ray diffraction quality.

### X-Ray Diffraction Data Collection

All data were collected at 100K at Advanced Light Source (Berkeley, Calif.) beam line 8.2.1, Advanced Photon Source (Chicago, Ill.) beam lines 24-ID-C and 24-ID-E, and in-house on a Rigaku Raxis-IV++ imaging plate detector using Cu K(alpha) radiation from a Rigaku FRE+ rotating anode generator with confocal optics (Table 2). Single crystals were mounted with CrystalCap HT Cryoloops (Hampton Research, Aliso Viejo, Calif.). K11V, K11VV2L, K11V-Br2, K11V-Br8, and K11V-TR crystals were flash frozen in liquid nitrogen prior to data collection. For experimental phases, K11V-TR crystals were soaked briefly in a mother liquor solution containing potassium iodide and flash frozen in liquid nitrogen. G6V crystals were cryoprotected in mother liquor solution containing 20% glycerol and flash frozen in liquid nitrogen.

### X-Ray Diffraction Data Processing and Refinement

All data were processed using DENZO (3) and SCALEPACK (3) or XDS (4). G6V initial phases were found by molecular replacement of a poly-alanine beta sheet template peptide. K11V-Br2 and K11V-Br8 were phased using HKL2MAP (5), and models built using COOT (6). K11V and K11VV2L were phased by molecular replacement using PHASER (7) with the K11V-Br2 structure. Low resolution (˜2.9 Å) experimental phases for K11V-TR was obtained from iodo soaked crystal diffraction data collected in-house using HKL2MAP (5), and followed by model building using COOT (6). All model refinement was done using REFMAC (8) and PHENIX (9).

### Surface Area Buried and Surface Complementarity Calculations

Surface area (10) and shape complementarity (11) calculations were performed with AREAIMOL and SC programs distributed by CCP4 (12).

### Native Nanoelectrospray Mass Spectrometry

Peak fractions containing the K11V-TR complex from SEC-HPLC in buffer (0.2M ammonium acetate, pH 6.5) were analyzed by direct nanospray injection (for review see (13)). Fractions were individually loaded into a 2-μm internal diameter externally coated nanospray emitter (ES380, Thermo) and desorbed by adjusting the spray voltage to maintain an ion current between 0.1 and 0.2 μA. A hybrid linear ion-trap/FTICR mass spectrometer was used for the analysis (7T, LTQ FT Ultra, Thermo Scientific, Bremen, Germany). Individual charge states of multiply protonated K11V-TR complex ions were selected for isolation and collisional activation in the linear ion trap followed by detection of the resulting product ions in the FTICR cell. Xtract software (Thermo Scientific, Bremen, Germany) was used to compute monoisotopic mass from the measured isotopomer profile.

### Dot Blot Analysis

Briefly, a small aliquot of cylindrin peptide samples, at a concentration of a few mg/mL, were spotted onto a nitrocellulose membrane (Trans-Blot, Bio-Rad, Hercules, Calif.). After blocking with 10% fat free milk in TBST buffer (50 mM Tris, 150 mM NaCl, 0.05% Tween20), the membranes were incubated with polyclonal antibody or monoclonal antibody (˜1:250 dilution in 5% fat free milk, TBST buffer) at room temperature for 1 hour. The membranes were washed three times in TBST buffer before incubating with anti-rabbit HRP-linked antibody (1:5000 dilution in 5% fat free milk, TBST buffer) (Invitrogen, Carlsbad, Calif.) at room temperature for 1 hour. After washing the membranes three times in TBST buffer, the films were developed following the protocol as described in the Kit (Thermo Scientific Pierce ECL Western Blotting Substrate, #32209). Positive controls for A11 and OC were prefibrillar oligomers and fibrils, respectively (14).

### Fibril Formation and Electron Microscopy

Fibrillation assays were initially carried out in fifteen different fibrillation conditions, then narrowed down to four conditions: A—phosphate buffered saline, B—25 mM TRIS pH 8.5, 150 mM sodium chloride, C—10% dimethyl sulfoxide (DMSO), 25 mM TRIS pH 8.5, 150 mM sodium chloride, and D—10 mM CAPS pH 11.0, 150 mM sodium chloride, 1 mM EDTA. Beta cylindrin peptides stock solutions (10 mg/mL in water) were diluted in a fibrillation buffer to a final concentration of 1 mg/mL in a microfuge. Samples were incubated at 50° C. with vigorous shaking (Torrey Pines Scientific, Carlsbad, Calif.) for one week. Most cylindrin peptides grew fibrils in buffer D, and some in buffers B-C. Fibrils did not appear in buffer A, but served as a negative control.

### Cell Culture and Viability Assay

Cell viability was investigated using a CellTiter 96 aqueous non-radioactive cell proliferation assay kit (MTT) (Promega cat. #G4100). SH-SY5Y (ATCC; cat. # CRL-2266), PC-12(ATCC; cat. # CRL-1721), HeLa and HEK293 were used to assess the toxic effect of clyindrin peptides. HeLa and HEK293 cells were cultured in DMEM medium with 10% fetal bovine serum. SH-SY5Y cells were cultured in F12/DMEM 1:1 medium with 10% fetal bovine serum, PC-12 cells were cultured in ATCC-formulated RPMI 1640 medium (ATCC; cat.#30-2001) with 10% heat-inactivated horse serum and 5% fetal bovine serum. Cells were maintained at 37° C. in 5% CO₂. For all toxicity experiments, 96-well plates (Costar cat. #3596) were used. HeLa, HEK293 and PC-12 cells were plated out at 10,000 cells per well and SH-SY5Y cells were plated at 25,000 cells per well. Cells were cultured for 20 h at 37° C. in 5% CO₂ prior to addition of peptide samples. 10 μl of sample was added to each well containing 90 μL medium, and allowed to incubate for 24 h prior to adding 15 μl Dye solution (Promega. cat. #G4102) into each well, followed by incubation for 4 h at 37° C. in 5% CO₂. After incubation, 100 μl solubilization Solution/Stop Mix (Promega cat. #G4101) was added to each well. After 12 h incubation at room temperature, the absorbance was measured at 570 nm. Background absorbance was recorded at 700 nm. Each of the experiments was repeated 3 times with 4 replicates per sample per concentration. The concentration for cylindrin peptides were based on their oligomeric state. That is a trimer for K11V-TR and monomer for K11VV4W-TR. Abeta at 0.5 μM was a positive control. The results were normalized by using the buffer treated cell as 100% viability and cell treated with 0.2% SDS as 0% viability.

### Preparation of Large Unilamellar Vesicles (LUVs)

Calcein-containing LUVs were prepared as described previously (19), with minor modifications. 1-palmitoyl-2-oleoyl-sn-glycero-3-phosphocholine (POPC) and 1-palmitoyl-2-oleoyl-sn glycero-3-phospho-L-glycerol (POPG) were obtained from Avanti Polar Lipids (Alabaster, Ala.). Mixtures of POPC and POPG in a 7:3 molar ratio were dissolved in 1:1 chloroform:methanol. The solvent was evaporated under dry nitrogen gas to yield a lipid film that was further dried under vacuum for at least 24 hours to remove any residual organic solvent. The film was then hydrated in 70 mM calcein (Sigma Aldrich, St. Louis, Mo.) and 10 mM Tris-HCl (pH 7.4) at a lipid concentration of 5 mM. The suspensions were subjected to 10 freeze-thaw cycles of temperatures of −80 and 50° C., followed by extrusion through two 0.2 μm pore size filters (Whatman, Florham Park, N.J.). Non-encapsulated calcein was separated from calcein-filled LUVs by size exclusion using a Sephadex G-75 (GE Healthcare, Piscataway, N.J.) equilibrated in buffer (10 mM Tris-HCl pH 7.4, 100 mM sodium chloride). The calcein-containing LUVs were concentrated to 3-5 mM and stored at 4° C. Phospholipid content and concentrations of the LUVs were determined by RP-HPLC using 100% Methanol and 100 mM TEAC (tetraethylammonium chloride) pH 7.8 as the solvent and a C18 column. Diameter of LUVs were determined using a Microtrac UPA 150 (York, Pa.). LUV preparations displayed diameters between 170-200 nm for at least 10 days. LUVs were used for experiments within 5 days of preparation.

### Membrane Leakage Experiments

Pure, lyophilized K11V-TR and K11VV2L-TR peptides were solubilized in water. Three different stock solutions were made at concentrations of 0.2, 0.8, and 2 mM. 5 uL of each peptide stock solution was added to a well containing 195 uL of 7:3 calcein-containing POPC:POPG LUVs in a buffer (10 mM Tris-HCL pH 7.4, 100 mM sodium chloride). Dye leakage was measured using a SpectraMax5 (Molecular Devices, Sunnyvale, Calif.) at 485 nm excitation and a 535 nm emission. Measurements were taken every 10 minutes for 30 hours. Each well contains a final LUV concentration of 100 μM with final K11V-TR and K11VV2L-TR construct concentrations of 5, 20, and 50 μM. Synthetic human Islet Amyloid Polypeptide (hIAPP) residues 8-37, purchased from CS-Bio (Menlo Park, Calif.), was used as a positive control; since hIAPP has been previously shown to interact and disrupt membranes (15, 16). Lyophilized hIAPP was dissolved in 100% hexafluoro-2-propanol (HFIP) and put under vacuum to evaporate the HFIP. hIAPP was then reconstituted in water at 0.2 mM, filtered using a 0.2 μm filter, and added to six wells containing 100 μM Calcein-containing LUVs to a final concentration of 5 μM hIAPP. Fluorescence at a given time point was normalized as described previously (20), using the equation: Fnormalized=(Ft−Fmin)/(Fmax−Fmin) Ft is the measured fluorescence intensity, Fmin the fluorescence of 100 μM calcein-containing LUVs alone, and Fmax is the maximum fluorescence determined by incubation of 100 μM LUVs in the presence of 0.5% Triton X-100.

### SOM Text

### Proposed and Similar Cylindrin Models

Considering conformational and geometrical properties of anti-parallel beta-sheets, Salemme and Weatherford attempted to model a six stranded barrel with a minimum of four interchain hydrogen bonds (17). They were unable to present a model, but observed one or more beta-bulges when maintaining beta-sheet twist, and a chain tendency to diverge at the ends of the barrel (17).

A Dali (18) and MATRAS (19) search was performed using the cylindrin as the search model to find similar structures. A cylindrin containing six chains, all with the same chain identification, was used to overcome the minimum chain length limit of 30 amino acids in Dali. The top scoring search result for Dali was alpha-amino acid ester hydrolase (PDB 1RRY) and for MATRAS was cytokine receptor common beta chain (PDB 1EGJ) residues 339-437. The Dali top result has a Z score of 2.7 and rmsd of 3.2, and MATRAS a Z score of 13.74. The MATRAS top scoring structure has the greater similarity, containing seven beta strands in a cylindrical type fashion. The Dali top scoring model has little similarity, and poor alignment to the alpha-amino acid ester hydrolase sequence, which may explain the low Z score of 2.7. Interestingly, a similar structure can be found in the PduU shell protein (PDB 3CGI), but not found using the above search engines. The PduU shell proteins N-termini form a six stranded parallel barrel (20). The parallel beta barrel, containing residues 7-17, has an Ab of 1050 Å2 and Sc of 0.76. Exhibiting similar molecular properties to the anti-parallel cylindrin structure, we classify this as a parallel cylindrin. A similar beta barrel with a shear number of 6 has been observed in the tetrahydrodipicolinate-N-succinyltransferase protein (PDB 1TDT) (21). This barrel is formed from C-terminal hairpins of 3 chains, adopting an antiparallel structure. But unlike the cylindrin, the interior seems to be quite polar, containing H-bonded rings of Asn and Ser sidechains and some waters.

### Cylindrin Crystal Packing

A different crystal packing was observed with the K11V-Br2 peptide, in which valine at position 2 is substituted by (2-bromoallyl)-glycine. In this derivatized structure, the asymmetric unit is two entire neighboring cylindrins. In contrast, cylindrins formed from the wildtype sequence K11V and the derivatized segment K11V-Br8, contain only one crystallographically unique cylindrin. The RMSD of the wildtype structure superimposed on the bromo-derivative structures is 0.76 and 0.37 Å for K11V-Br2 and K11V-Br8, respectively; RMSD of the wild-type backbone on the bromo-derivative backbones is 0.33 and 0.29 Å for K11V-Br2 and K11V-Br8, respectively.

### Molecular Dynamic Simulations and Calculations

Molecular dynamics (MD) simulations were carried out to examine the structural transition pathway and the energetics associated with the conversion between the native conformation (cylindrin; FIG. S9A) and a cylindrin fibril model (discussed below; FIG. S9B). These two models are the two target structures used in the molecular simulation. NAMD software was used to integrate classical equations of motion of model systems (22), and the Charmm22 all-atom force field with CMAP correction (23) was used. Van der Waals and electrostatic interactions were switched with a 12 Å cutoff distance. The SHAKE algorithm constrained the covalent bond length of a polar hydrogen atom to its donor, which enabled 2 fs integration step. The native model was solvated with TIP3 explicit water molecules in an 80 Å³ cubic box. Initially each target conformation was energy minimized in 1200 steps, heated to 300 K in 100 ps and equilibrated in 500 ps by rescaling temperature periodically. During the equilibration and the production period, Langevin piston algorithm controlled the pressure at 1 atm and the temperature at 300 K. Next, we adopted targeted MD (TMD) simulation (24) to elucidate intermediate conformations in the middle of the transition pathway. After the equilibrating period, cylindrin was gradually transformed to the fibril model in 20 ns by applying a constraining potential on Cα atoms (forward simulation);

U(\vec{x},t)=½k(R(\vec{x})−R*(t))²

where [symbol] is the coordinate vector of Cα atoms, t is the current simulation time, k is strength of the constraint which was set to 20 kcal/mol/A R( [symbol] ) is the root-mean-squared deviation (RMSD) of Cα atoms to the β-sheet, and R*(t) is the target RMSD at t which was linearly reduced from the initial RMSD between two end structures (10.04 Å) to zero as the simulation progressed. We also performed a reverse TMD simulation starting from the last snapshot of the forward simulation, and gradually transformed the molecule to cylindrin (backward simulation). The TMD simulations successfully converted the cylindrin to the fibril and vice versa.

TMD simulation is susceptible to hysteresis effect in energy changes which hampers accurate estimation of free energy difference and the transition state energy between two end structures (24). Therefore we employed free energy perturbation (FEP) simulation aimed at an accurate estimation of the free energy change associated with the structural conversion from the cylindrin to the fibril model (25, 26). The relative difference in RMSD (ΔRMSD) of the two end structures was chosen as the reaction coordinate of the transition. The reaction coordinate varied from −10.0 Å to +10.0 Å, which was divided by 40 equally spaced windows. Initial conformers for the FEP simulation were chosen from the previous TMD simulation; for each window iε{n|1δnδ 40}, two of lowest energy conformation from each of the forward and the backward simulation were selected. We applied an umbrella potential to each initial conformation, whose energy minimum was located at the center of the window;

$${U}_{i} ( {x}^{r} , t ) = \frac{1}{2} {k ( {R}_{1} ( {x}^{r} ) - {R}_{2} ( {x}^{r} ) - {R}_{i}^{*} )}^{2}$$

where i is the index of windows, R₁( [symbol] ) and R₂( [symbol] ) are RMSD of Cα atoms to cylindrin and to the fibril model respectively, k is 20 kcal/mol/Å², and Rᵢ* is the offset of the umbrella potential aligned with the center of each bin. For each window, the initial conformation was heated to 300 K in 100 ps, while employing a harmonic constraint to Cα atoms to prevent abrupt structural changes. The Langevin piston algorithm was applied to maintain pressure at 1 atm with a temperature of 300 K. During the production period, the offset of the umbrella potential was shifted from i to i+1, i+1 to i+2, i to i−1 and i−1 to i−2 positions respectively. Simulation period at each offset value varied from 0.75 to 1.5 ns depending on convergence of the simulation. The total energy, constraining energy, and reaction coordinate were saved every 0.2 ps, and coordinates were saved every 2.0 ps.

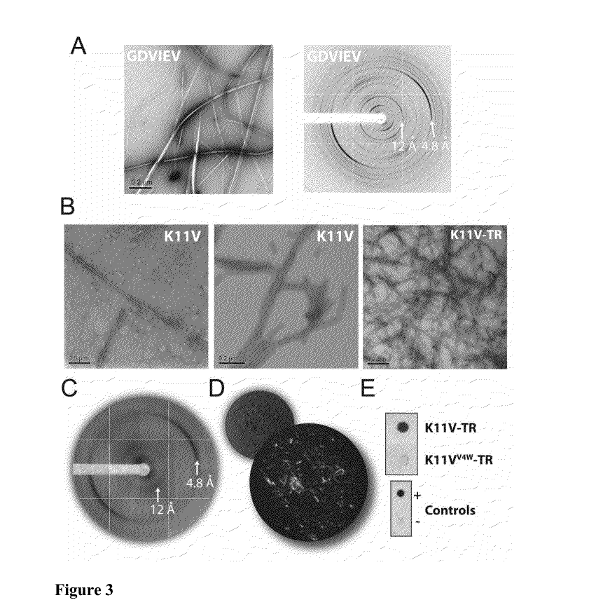

After finishing the FEP simulation we generated an energy histogram of the entire FEP simulation, using weighted histogram analysis method (WHAM) (27). The energy density of state (DOS) information was utilized to compute Gibbs free-energy of each window along the reaction coordinate (ΔRMSD), which was plotted in FIG. 3A. The free energy of a reaction coordinate window (i) is defined:

$${F}_{i} = {\sum }_{α ∈ \{ i \}} Ω ( {E}_{α} + {U}_{α} ) {e}^{- β {E}_{α}} dE$$

where α is index of snapshots, E and dE are discretized energy level and its width (2 kcal/mol), Ω is energy density of state, and Ua is constraining potential of the window. In addition, a hierarchical clustering algorithm was used to define a representative conformation of each reaction coordinate window. We used a Cα RMSD distance of 3.0 Å as the cutoff for defining a structural cluster. The first 3000 snapshots were analyzed for lowest free energy within each window. A representative configuration in the most populated cluster is plotted in FIG. 3A. For free energy and clustering analyses, we used the MD Analysis package (28) and the Scipy-cluster package (29).

The total free energy of cylindrin and the steric-zipper fibril model were compared within the Molecular Mechanics-Generalized Born/Surface Area approximation method (MM-GB/SA) (30). The Generalized-Born solvation model and the surface area dependent hydrophobic energy were incorporated as functions of the solvation effect. This technique has been successfully applied for comparing the energetic stability of amyloid fiber models (31). The cylindrin steric-zipper fibril model, consisted of a steric-zipper interface wherein the hydrophobic residues (Val 2, Val 4, and Val 8) buried within the bilayer forming the dehydrated interface (FIG. S10). This model was solvated in a tetragonal solvation box (29.15×100×100 Å³). The X dimension of the solvation box was aligned parallel to the fiber axis, as to represent an infinitely long fiber. The model was heated and equilibrated in 600 ps at 300 K, and simulated for 10 ns without structural constraints. The coordinates were saved every 2 ps. The steric-zpper interface was intact during the simulation period. In contrast, the cylindrin was solvated in an 80×80×80 Å³ solvation box, and simulated for 10 ns. After completion of MD simulations, simulated snapshots were analyzed without solvent molecules and analyzed using an implementation of Generalized-Born solvation model in Charmm v31 (32, 33). In the GBSA approximation, the total free energy of a molecule is sum of individual contributions (30);

E_{Total}=Eᵢₙₜ+E_{vdW}+E_{Elec}+E_{GB}+E_{ASP},

where Eᵢₙₜ is summation of covalent bonding energy terms (bond, angle, dihedral, improper dihedral, and CMAP correction), E_{vdW} is Van der Waals energy, E_{Elec} is vacuum electrostatic energy, E_{GB} is Generalized-Born solvation energy, E_{ASP} is surface area dependent hydrophobic energy, with a surface tension coefficient σ=5 cal/mol/Å², S_{Trans} is translational entropy, and S_{Rot} is rotational entropy. The trans-rotational entropy of the steric-zipper fibril was set to zero, since it precipitates in vitro. Unlike the original method, the vibrational entropic contribution was ignored which contributes only a small fraction to the total energy (31). The density of cylindrin was set to 1 mM/L, and any change in this density did not affect our conclusion qualitatively; for example, when the density is set to 1 nM/L resulted in −TS_{Trans}=−4.83 kcal/mol. The MM-GB/SA analysis determined the steric-zipper fibril model has −5.2 kcal/mol/peptide lower free energy than the cylindrin (Table 4).

TABLE 4
Results of GBSA calculations for cylindrin and fibril models at
300K. The energy unit is kcal/mol/peptide.

| Structure | | | | | | | | |
|---|---|---|---|---|---|---|---|---|
| Type | Eᵢₙₜ | E_{vdW} | E_{Elec} | E_{GB} | E_{ASP} | −TS_{Trans} | −TS_{Rot} | Total |
| cylindrin | 164.8 | −39.4 | −179.7 | −254.8 | 4.8 | −3.68 | −2.58 | −310.6 |
| fibril | 172.6 | −52.1 | −410.6 | −29.4 | 3.8 | 0 | 0 | −315.7 |

### Potential Cylindrin A11 Epitopes

Because the polyclonal A11 antibody was affinity purified on an AB containing matrix (Kayed et al. Conformation-dependent anti-amyloid oligomer antibodies. Methods Enzymol. 2006; 413:326-44. PubMed PMID: 17046404), cylindrin and AB prefibrillar oligomers presumably share an epitope(s) that is also shared by other toxic oligomers which the A11 antibody recognizes. Several structural features are shared by our current models of cylindrin. They are the radius of the cylindrin, water mediated backbone H-bonds at the ends of the cylindrin, and helical grooves between side chains on the outside surface of the cylindrin. These grooves are akin to the linear grooves between side chains on the outside surface of steric zippers, but are more pronounced because the cylindrin side chains project from a convex surface of the cylindrin.

### Fibril Model of the Cylindrin Sequence

Fourier transform infrared spectroscopy of cylindrin, K11V-TR dried fibrils display anti-parallel beta sheet characteristics. In the FTIR spectrum (data not shown) we observed peaks at 1628 cm-1 and 1685 cm-1, characteristic of intermolecular and anti-parallel beta sheet (34, 35), respectively. Therefore, an anti-parallel model for cylindrin fibrils was constructed similar to that observed for short steric zippers (36), and subsequently used in targeted molecular dynamics simulation (discussed above).

Results of GBSA calculations for cylindrin and fibril models at 300 K. The energy unit is kcal/mol/peptide.

| Structure | | | | | | | | |
|---|---|---|---|---|---|---|---|---|
| Type | Eᵢₙₜ | E_{vdW} | E_{Elec} | E_{GB} | E_{ASP} | −TS_{Trans} | −TS_{Rot} | Total |
| cylindrin | 164.8 | −39.4 | −179.7 | −254.8 | 4.8 | −3.68 | −2.58 | −310.6 |
| fibril | 172.6 | −52.1 | −410.6 | −29.4 | 3.8 | 0 | 0 | −315.7 |

### Potential Cylindrin A11 Epitopes

Because the polyclonal A11 antibody was affinity purified on an AB containing matrix (Kayed et al. Conformation-dependent anti-amyloid oligomer antibodies. Methods Enzymol. 2006; 413:326-44. PubMed PMID: 17046404), cylindrin and AB prefibrillar oligomers presumably share an epitope(s) that is also shared by other toxic oligomers which the A11 antibody recognizes. Several structural features are shared by our current models of cylindrin. They are the radius of the cylindrin, water mediated backbone H-bonds at the ends of the cylindrin, and helical grooves between side chains on the outside surface of the cylindrin. These grooves are akin to the linear grooves between side chains on the outside surface of steric zippers, but are more pronounced because the cylindrin side chains project from a convex surface of the cylindrin.

### Fibril Model of the Cylindrin Sequence

Fourier transform infrared spectroscopy of cylindrin, K11V-TR dried fibrils display anti-parallel beta sheet characteristics. In the FTIR spectrum (data not shown) we observed peaks at 1628 cm-1 and 1685 cm-1, characteristic of intermolecular and anti-parallel beta sheet (34, 35), respectively. Therefore, an anti-parallel model for cylindrin fibrils was constructed similar to that observed for short steric zippers (36), and subsequently used in targeted molecular dynamics simulation (discussed above).

### B. Results

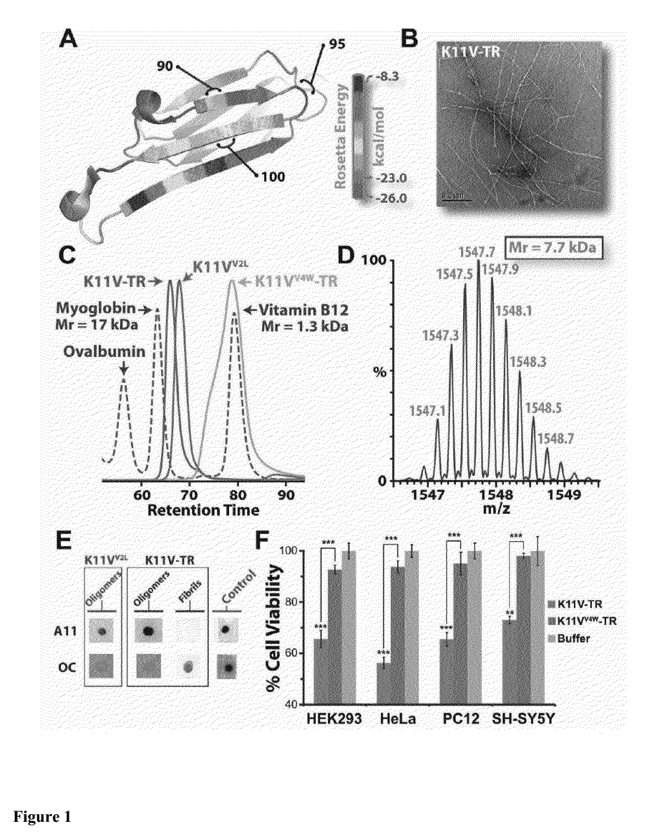

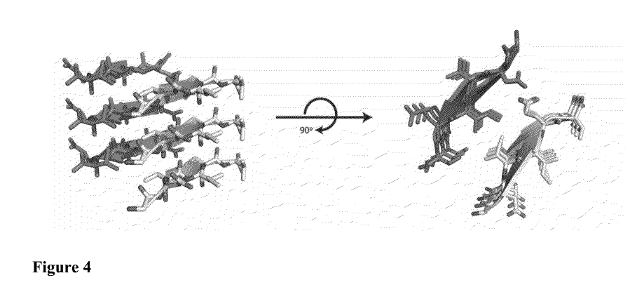

We identified the oligomer-forming segment of ABC by inspection of its 3D structure (16) and by applying the Rosetta-Profile algorithm to its sequence. This algorithm identifies sequence segments that form the steric-zipper spines of amyloid fibrils (17, 18). We noted that two segments of high amyloidogenic propensity, with sequences KVKVLG (SEQ ID NO:1) and GDVIEV (SEQ ID NO:2), share the same Gly residue 95 at the C-terminus of the first segment and the N-terminus of the second; moreover, the entire 11-residue segment KVKVLGDVIEV (SEQ ID NO:3) forms a hairpin loop in the 3D structure of ABC (FIG. 1A), with Gly at its center. As predicted, the second six-residue segment GDVIEV (SEQ ID NO:2), termed G6V (see Table I which defines the structures described in this report), forms fibrils and microcrystals (FIG. 3). The microcrystals enabled us to determine the atomic structure of G6V (FIG. 4), which proved to be a standard Class 2 steric zipper (19), essentially an amyloid-like protofilament.

The hairpin, segment KVKVLGDVIEV (SEQ ID NO:3) (termed K11V) formed both amyloid fibrils and oligomers. Upon shaking at elevated temperature, K11V forms fibrils similar to those of the parent protein (ABC) from which the segment is derived (15) and similar to those of a tandem repeat of K11V (K11V-TR) (FIG. 1B, FIG. 3B-C, and Table 1). The fibrils range from 20 to 100 nm in diameter as viewed by electron microscopy (FIG. 3). X-ray diffraction of dried fibrils displayed rings at 4.8 and 12 Å resolution, consistent with the signature cross-beta pattern of amyloid fibrils (FIG. 3C). The amyloid fibrils of K11V-TR bind the specific amyloid dye congo-red, producing apple-green birefringence under polarized light (FIG. 3D), and are immunoreactive with the fibril-specific, conformation-dependent antibody, OC (FIG. 1E and FIG. 3E) (20). Together these results prove that the segments G6V, K11V, and K11V-TR are all capable of converting to the amyloid state (21, 22), as is their parent protein, ABC.

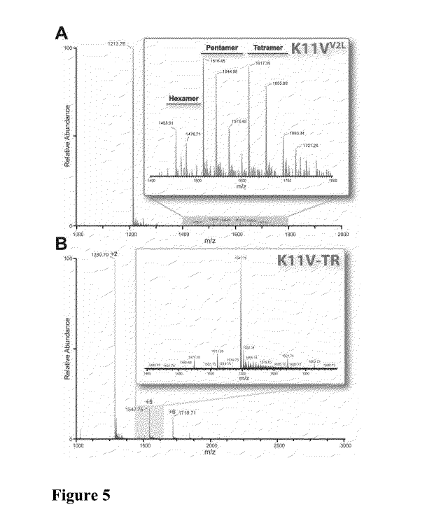

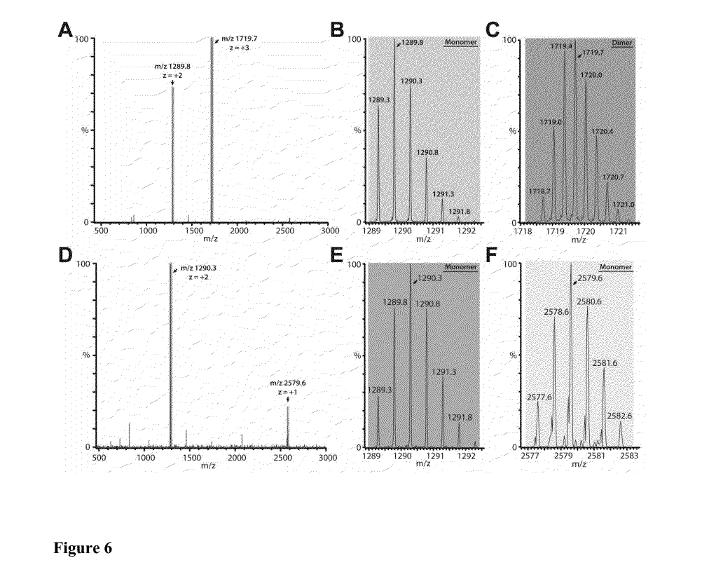

Under physiological conditions, the segment K11V, K11V-TR, and a sequence variant with Leu replacing Val at position 2 (K11VV2L), all form stable small oligomers, intermediate in size between monomer and fiber. For each sequence, we determined the number of molecules in the oligomers by size exclusion chromatography (SEC-HPLC) and native mass spectrometry experiments. Purified recombinant K11VV2L, and K11V-TR, a tandem repeat of K11VV2L eluted as oligomeric complexes by SEC (FIG. 1C). For example, the K11V-TR complex was estimated to be ˜8 kDa in mass, corresponding roughly to three tandem segment chains. As an additional check on the stoichiometry of the tandem repeat K11V-TR oligomer, we subjected peak fractions to native nanoelectrospray mass spectrometry. Mass spectra clearly showed abundant ions of K11V-TR oligomers with masses corresponding to three peptide chains (FIGS. 1D and 5). Furthermore, we were able to isolate ions of the K11V-TR oligomer and perform collision induced dissociation (CID) of this trimeric peptide complex into monomeric units of mass equal to the K11V-TR peptide (FIG. 6). Similar experiments show that K11V and K11VV2L form hexameric oligomers (Table 1 and FIG. 5). Thus native mass spectrometry is consistent with SEC-HPLC in suggesting a stoichiometry of a K11V oligomer of six chains and a K11V-TR oligomer of three tandem chains. These results are consistent with crystallography and energetic considerations (see below).

TABLE 1
Cylindrin single chain and tandem repeat peptide
abbreviations and amino acid sequences.

| Peptide Abbreviation | Peptide Sequence |
|---|---|
| G6V | GDVIEV |
| | (SEQ ID NO: 2) |
| K11V | KVKVLGDVIEV |
| | (SEQ ID NO: 3) |
| K11V-Br2 | KBKVLGDVIEV |
| | (SEQ ID NO: 4) |
| K11V-Br8 | KVKVLGDVBEV |
| | (SEQ ID NO: 5) |
| K11VV2L | KLKVLGDVIEV |
| | (SEQ ID NO: 6) |
| K11V-TR | GKVKVLGDVIEVGGKVKVLGDVIEV |
| | (SEQ ID NO: 7) |
| K11VV2L-TR* | GKLKVLGDVIEVGGKLKVLGDVIEV |
| | (SEQ ID NO: 8) |
| K11VV4W-TR | GKLKWLGDVIEVGGKLKWLGDVIEV |
| | (SEQ ID NO: 9) |

B-residue substitution with (2-bromoallyl)-glycine a non-natural amino acid;
*This peptide sequence has been denoted as K11V-TR in the text.

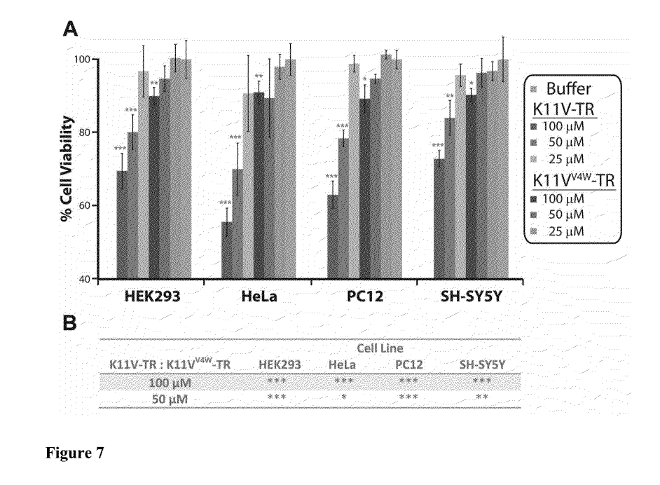

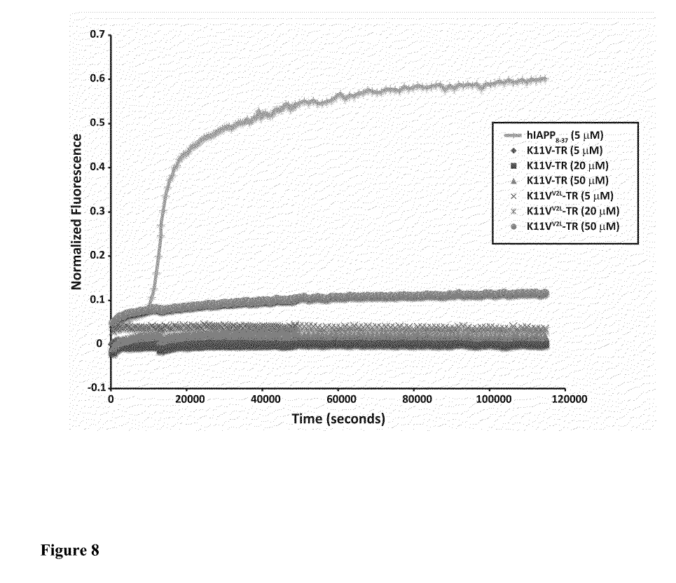

These ABC K11V oligomers exhibit molecular properties in common with amyloid oligomers from other disease-related proteins. We probed blots of the recombinant segments with the polyclonal A11, amyloid-oligomer-specific conformational antibody (5). Both single and tandem repeat segments are recognized by the A11 antibody (FIG. 1E and FIG. 3E). Using a cell viability assay on mammalian cells, we observed oligomers to be toxic, displaying dose-response effects similar to those of Abeta involved in Alzheimer's disease (2, 23, 24) (FIG. 1F and FIG. 7). To test if membrane disruption is responsible for this toxicity, as suggested for human Islet Amyloid Polypeptide (hIAPP) (25, 26), we performed liposome dye-release experiments. The hIAPP peptide clearly diminished liposome integrity leading to dye release, but the K11V-TR did not exhibit this trend (FIG. 8). In contrast to oligomeric solutions, no toxicity was observed for the fibrils of G6V. Thus ABC segments in oligomeric form are cytotoxic, but suggest a more complicated mechanism of toxicity than membrane disruption.

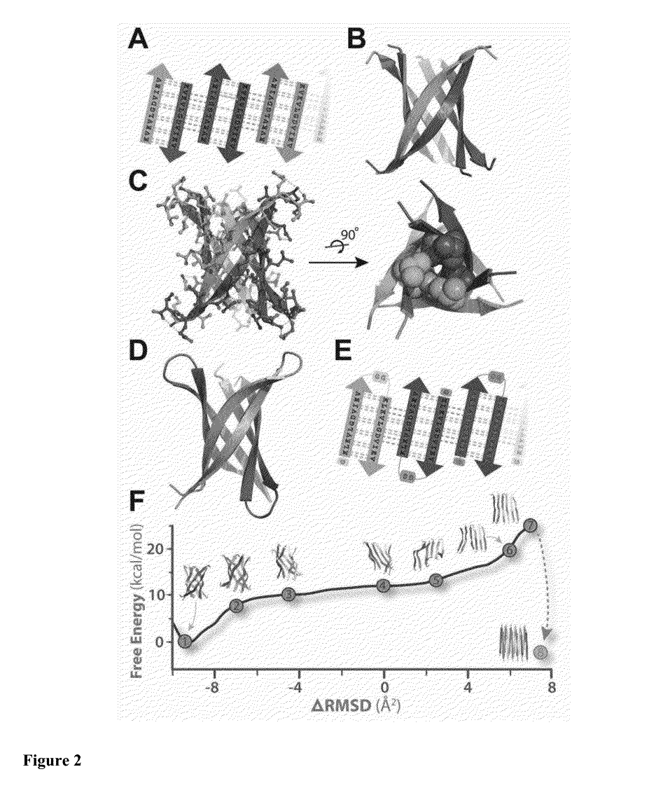

We next determined the crystal structures of various ABC K11V oligomers. A screen produced X-ray grade crystals of K11V, but structure determination by molecular replacement with fiber-like probes failed, suggesting that the ABC segment oligomers possess a previously unobserved type of amyloid structure. Turning to the SAD method for phase determination, we synthesized K11V derivatives with Br substitutions at positions 2 or 8 of the K11V sequence, K11V-Br2 and K11V-Br8, with the leucine-resembling non-natural amino acid (2-bromoallyl)-glycine. Both derivatives crystallized and led to structure determinations (Table 2) at 1.4 Å resolution. Molecular replacement based on these structures led to the closely related structures of K11V itself, as well as K11V-TR and K11VV2L The structure of K11V, the amyloid-related oligomer, is a six-stranded anti-parallel barrel of cylindrical shape, consistent in mass with our solution studies, which we term a cylindrin. The cylindrin (FIG. 2) is distinctly different in structure from either the native structure of ABC (FIG. 1A) or from its G6V segment (FIG. 4), a dual beta-sheet steric zipper. It is also distinct from other structures currently in the Protein Data Bank (see SOM Text, Proposed and Similar Cylindrin Models), but resembles several previously proposed beta-barrel models (27-31). Each strand of the cylindrin is bonded to one neighboring strand by a strong interface and to a second by a weak interface. The strong interface (between purple and green chains, FIG. 2B-C) is formed by twelve hydrogen bonds, and splays outward at the ends. The weak interface is formed by eight hydrogen bonds, four from the main-chain, two mediated through sidechain interactions, and two through a water bridge (FIG. 2C). The axial channel of the cylindrin is closed by the hydrophobic interactions of two inward pointing sets of three valine sidechains, and is devoid of water (FIG. 2C). The surface area buried per residue (Ab) in the strand packing interface of the cylindrin is 87 Å2, smaller than the 131 Å2 value for the strand-to-strand interface of the steric zipper of GNNQQNY (SEQ ID NO:10) (32). Similarly the cylindrin packing interface has a shape complementarity (Sc) value of 0.75, somewhat smaller than the value of 0.80 for the GNNQQNY interface (Table 3). Thus the cylindrin structure has features in common with a steric zipper in being formed from hydrogen bonded beta-strands and having a dry interior, but it is cylindrical rather than nearly flat, and is probably less stable, as suggested by the lower Ab and Sc values.

TABLE 2
X-ray Data Collection and Refinement Statistics ^{a}.

| | K11V | K11V-Br2^{b} | K11V-Br8^{b} | K11V^{V2L} | K11V-TR | GDVIEV |
|---|---|---|---|---|---|---|
| Data Collection | | | | | | |
| Synchrotron Beam line | APS 24-ID-C | ALS 8.2.1 | APS 24-ID-C | APS 24-ID-C | APS 24-ID-C | APS 24-ID-E |
| Reflections observed | 7,715 | 21,693 | 10,088 | 84,426 | 23,465 | 2,148 |
| Unique reflections | 973 | 6,043 | 1,509 | 9,431 | 6,428 | 438 |
| Wavelength (Å) | 0.97918 | 1.00 | 0.91963 | 0.9794 | 0.97918 | 0.97915 |
| Resolution (Å) | 2.55 | 1.62 | 2.8 | 1.4 | 2.16 | 1.7 |
| Highest Resolution Shell (Å) | 2.64-2.55 | 1.68-1.62 | 2.9-2.8 | 1.6-1.4 | 2.21-2.16 | 1.83-1.7 |
| Space Group | I4₁32 | I2₁3 | I2₁3 | I2₁3 | P6₁ | P2₁ |
| R_{sym} (%) ^{c} | 7.5 (60) | 3.3 (28.6) | 9.0 (66.3) | 7.2 (49.9) | 4.5 (41.6) | 17.8 (38.6) |
| l/σ | 16.8 (1.6) | 25.0 (3.2) | 15.9 (2.5) | 25.3 (3.1) | 14 (3.7) | 5.1 (5.0) |
| Completeness (%) | 92 (98) | 97.2 (98.7) | 100 (100) | 99.8 (100) | 99.3 (4.7) | 97.6 (100) |
| Unit cell dimensions | | | | | | |
| a, b, c (Å) | 69.2, 69.2, 69.2 | 65.9, 65.9, 65.9 | 70.3, 70.3, 70.3 | 65.8, 65.8, 65.8 | 52.34, 52.34, 87.33 | 4.8, 19.5, 21.0 |
| α, β, λ (°) | 90, 90, 90 | 90, 90, 90 | 90, 90, 90 | 90, 90, 90 | 90, 90, 120 | 90, 94.2, 90 |
| Refinement | | | | | | |
| Resolution (Å) | 48-2.5 | 33-1.7 | 49-2.8 | 46-1.4 | 19.7-2.17 | 14-1.7 |
| Reflections Used | 914 | 5,409 | 1,373 | 9,412 | 6,421 | 389 |
| R_{work} (%) | 24.4 (38.5) | 18.7 (17.1) | 23.3 (24.5) | 17.8 (19.7) | 18.4 (22.0) | 21.23 (29.2) |
| R_{free} (%) | 26.9 (46.8) ^{d} | 22.9 ^{d} | 23.6 (37.4) ^{d} | 24.0 (22.6) ^{d} | 23.4 (25.5) ^{e} | 22.4 (20.1) ^{e} |
| Peptides in Asymmetric Unit | 1 | 4 | 2 | 4 | 6 | 1 |
| Number of non-H atoms | | | | | | |
| Protein | 84 | 345 | 170 | 404 | 1,078 | 46 |
| Non-protein | 1 | 27 | — | 54 | 86 | 2 |
| RMS deviations | | | | | | |
| Bond lengths (Å) | 0.022 | 0.012 | 0.016 | 0.007 | 0.01 | 0.011 |
| Bond angles (°) | 1.7 | 1.5 | 17 | 1.1 | 1.4 | 1.1 |
| Average B-factor (Å²) | | | | | | |
| Protein atoms | 40.8 | 20.6 | 71.0 | 19.6 | 63.8 | 9.2 |
| Non-protein atoms | 33.8 | 37.2 | — | 38.1 | 74.0 | 25.4 |
| PDB accession code | 3SGO | 3SGM | 3SGN | 3SGP | 3SGR | 3SGS |

^{a} Highest resolution shell shown in parenthesis.
^{b}Number corresponds to position of (2-Bromoallyl)Glycine residue substitution in eleven amino acid peptide sequence, see Table 1.
^{c} R_{sym} = Σ | I-<I> | / ΣI.
^{d} Rfree calculated using 5% of the data.
^{e} Rfree calculated using 10% of the data.

TABLE 3
Surface area buried (SA) and surface complementarity
(Sc) for cylindrin, G6V, and Sup35 peptide segment,
GNNQQNY. Values for the fibril were calculated by
using one chain buried within the extended fibril.

| | | | | SA/ |
|---|---|---|---|---|
| Peptide Segment | Structure Type | S_{c} | SA | residue |
| K11V | Cylindrin | 0.75 | 959 | 87 |
| G6V | Two interacting | 0.82 | 112 | 19 |
| | strands | | | |
| | Fibril | 0.72 | 623 | 104 |
| GNNQQNY | Two interacting | 0.86/0.80* | 147/157* | 25/26* |
| (SEQ ID NO: 12) | strands | | | |
| | Fibril | 0.82 | 787 | 131 |

*From Sawaya, et al. 2007 (36).

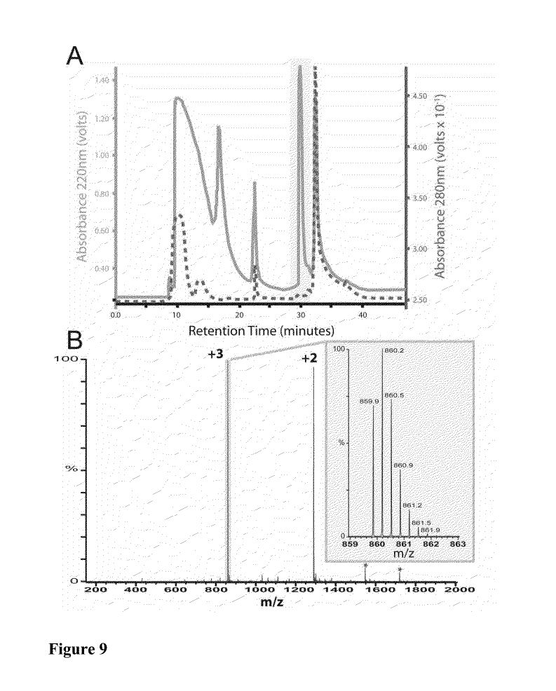

To provide adequate cylindrin material for biochemical and toxicological studies, we generated a synthetic gene to express in bacteria a tandem repeat, K11V-TR, of the well diffracting K11VV2L segment, covalently linked through a double glycine linker and containing an additional N-terminal glycine (FIG. 9 and Table 1). This K11V-TR peptide reduces the complexity of the cylindrin assembly process from six to three chains (FIG. 2D-E). We were able to determine the K11V-TR crystal structure, even though the glycine linkers produce some disorder in the crystals (Table 2). Other than the glycine linkers and the Val to Leu replacement, the cylindrical bodies of the six-stranded K11V and the three double-stranded K11V-TR oligomers are essentially identical. Energetic considerations suggest that the cylindrin should be stable in solution: the surface area buried per interchain interface of K11V-TR is 841 Å2, nearly as much as for the foldon trimerization domain, 1092 Å2 (PDB 1RFO), and cylindrin forms twice as many hydrogen bonds between neighboring chains as does the foldon domain.

For a negative control of cylindrin structure and properties, we generated a variant form of the tandem segment, K11VV4W-TR in which the V4W substitution occurs in both repeats (Table 1). This substitution was predicted on the basis of the K11V crystal structure to disrupt oligomer formation through steric clash of core, buried residues. This variant peptide eluted in the mass range of a dimeric/monomeric species by SEC-HPLC and displayed dramatically reduced cell toxicity (FIGS. 1F and 7).

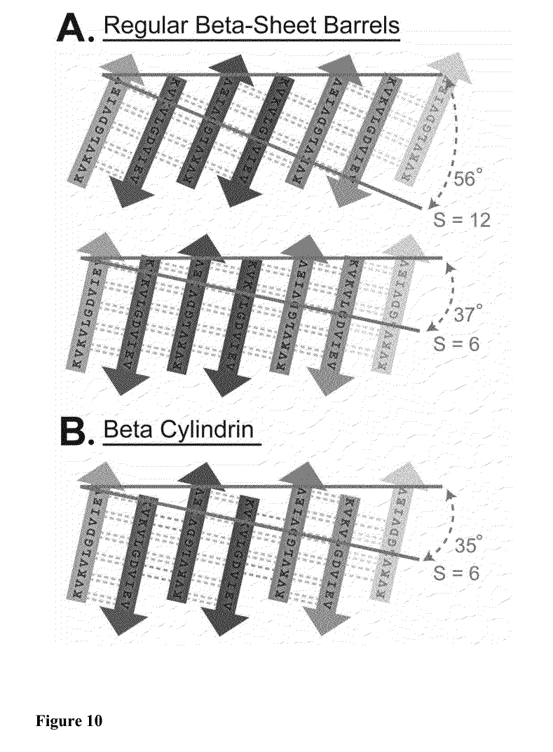

To compare cylindrins to fibers, we consider a cylindrin to be a toxic, amyloid-related, oligomeric, cylindrically shaped beta-barrel formed from anti-parallel, extended protein strands and having the cylinder filled with packed sidechains. A cylindrin resembles a steric zipper in having a packed interior, but differs from a steric zipper in an important respect which may illuminate the reaction pathway from oligomers to fibrils. When unrolled into a beta-sheet, each anti-parallel pair of strands in the cylindrin sheet (FIG. 2A) is out of register with neighboring pairs by 6 residues (shear number is 6) (FIG. 10) (33). In contrast, the beta-strands in full amyloid fibers (22, 34, 35) and short steric-zippers (19) are in-register. This means that a cylindrin unrolled into a sheet, would not be an in-register structure, ready to bond with an identical sheet to form the steric zipper spine of an amyloid fiber. The transition from cylindrin to steric zipper involves breaking of hydrogen bonds, and re-registration of the strands into an in-register structure, as we have simulated by targeted molecular dynamics, followed by free energy perturbation in explicit solvent (FIG. 2F and SOM Text, Molecular Dynamic Simulations and Calculations). We chose the end target as an antiparallel sheet, based on FTIR experiments (SOM Text, Fibril Model of the Cylindrin Sequence). These calculations suggest that the transition from cylindrin to an anti-parallel fiber-like structure involves crossing a high free energy implying that fibers may nucleate from monomers without passing through cylindrin-like oligomeric states (36-38); that is, cylindrin is likely to be off-pathway to fiber formation.

The atomic coordinates of ABC cylindrin are shown in Table 5.

TABLE 5

| | | | |
|---|---|---|---|
| HEADER | PROTEIN FIBRIL | 15-JUN-11 | 3SGO |

| | | |
|---|---|---|
| TITLE | | AMYLOID-RELATED SEGMENT OF ALPHAB-CRYSTALLIN RESIDUES 90-100 |
| COMPND | | MOL_ID: 1; |
| COMPND | 2 | MOLECULE: ALPHA-CRYSTALLIN B CHAIN; |
| COMPND | 3 | CHAIN: A; |
| COMPND | 4 | SYNONYM: ALPHA(B)-CRYSTALLIN, HEAT SHOCK PROTEIN BETA-5, HSPB5, |
| RENAL | | |
| COMPND | 5 | CARCINOMA ANTIGEN NY-REN-27, ROSENTHAL FIBER COMPONENT; |
| COMPND | 6 | ENGINEERED: YES |
| SOURCE | | MOL_ID: 1; |
| SOURCE | 2 | SYNTHETIC: YES; |
| SOURCE | 3 | ORGANISM_SCIENTIFIC: HOMO SAPIENS; |
| SOURCE | 4 | ORGANISM_COMMON: HUMAN; |
| SOURCE | 5 | ORGANISM_TAXID: 9606; |
| SOURCE | 6 | OTHER_DETAILS: SYNTHETIC PEPTIDE |
| KEYWDS | | AMYLOID, AMYLOID OLIGOMER, BETA CYLINDRIN, PROTEIN FIBRIL |
| EXPDTA | | X-RAY DIFFRACTION |
| AUTHOR | | A. LAGANOWSKY, M.R. SAWAYA, D. CASCIO, D. EISENBERG |
| REVDAT | 1 | 21-MAR-12 3SGO 0 |
| JRNL | | AUTH |

A. LAGANOWSKY, C. LIU, M.R. SAWAYA, J.P. WHITELEGGE, J. PARK, M. ZHAO,

| | |
|---|---|
| JRNL | AUTH 2 |

A. PENSALFINI, A.B. SORIAGA, M. LANDAU, P.K. TENG, D. CASCIO, C. GLABE,

| JRNL | AUTH 3 D. EISENBERG | | |
|---|---|---|---|
| JRNL | TITL | ATOMIC VIEW OF A TOXIC AMYLOID SMALL OLIGOMER. | |
| JRNL | REF | SCIENCE | V. 335 1228 2012 |

| JRNL | REFN ISSN 0036-8075 | |
|---|---|---|
| JRNL | PMID | 22403391 |
| JRNL | DOI | 10.1126/SCIENCE.1213151 |

| REMARK | 2 | | | | |
|---|---|---|---|---|---|
| REMARK | 2 | RESOLUTION. 2.56 ANGSTROMS. | | | |
| REMARK | 3 | | | | |
| REMARK | 3 | REFINEMENT. | | | |
| REMARK | 3 | PROGRAM : REFMAC 5.4.0061 | | | |
| REMARK | 3 | AUTHORS : MURSHUDOV, VAGIN, DODSON | | | |
| REMARK | 3 | | | | |
| REMARK | 3 | REFINEMENT TARGET : MAXIMUM LIKELIHOOD | | | |
| REMARK | 3 | | | | |
| REMARK | 3 | DATA USED IN REFINEMENT. | | | |
| REMARK | 3 | RESOLUTION RANGE HIGH | (ANGSTROMS) | : | 2.56 |
| REMARK | 3 | RESOLUTION RANGE LOW | (ANGSTROMS) | : | 48.97 |
| REMARK | 3 | DATA CUTOFF | (SIGMA(F)) | : | 0.000 |
| REMARK | 3 | COMPLETENESS FOR RANGE | (%) | : | 92.7 |
| REMARK | 3 | NUMBER OF REFLECTIONS | | : | 972 |
| REMARK | 3 | | | | |
| REMARK | 3 | FIT TO DATA USED IN REFINEMENT. | | | |
| REMARK | 3 | CROSS-VALIDATION METHOD | | : | THROUGHOUT |
| REMARK | 3 | FREE R VALUE TEST SET SELECTION | | : | RANDOM |
| REMARK | 3 | R VALUE (WORKING + TEST SET) | | : | 0.245 |
| REMARK | 3 | R VALUE (WORKING SET) | | : | 0.244 |
| REMARK | 3 | FREE R VALUE | | : | 0.270 |
| REMARK | 3 | FREE R VALUE TEST SET SIZE | (%) | : | 6.000 |
| REMARK | 3 | FREE R VALUE TEST SET COUNT | | : | 58 |
| REMARK | 3 | | | | |
| REMARK | 3 | FIT IN THE HIGHEST RESOLUTION BIN. | | | |
| REMARK | 3 | TOTAL NUMBER OF BINS USED | | : | 20 |
| REMARK | 3 | BIN RESOLUTION RANGE HIGH | (A) | : | 2.56 |
| REMARK | 3 | BIN RESOLUTION RANGE LOW | (A) | : | 2.62 |
| REMARK | 3 | REFLECTION IN BIN (WORKING SET) | | : | 73 |
| REMARK | 3 | BIN COMPLETENESS (WORKING + TEST) | (%) | : | 98.72 |
| REMARK | 3 | BIN R VALUE (WORKING SET) | | : | 0.3850 |
| REMARK | 3 | BIN FREE R VALUE SET COUNT | | : | 4 |
| REMARK | 3 | BIN FREE R VALUE | | : | 0.4680 |
| REMARK | 3 | | | | |

| REMARK | 3 | NUMBER OF NON-HYDROGEN ATOMS USED IN REFINEMENT. | | |
|---|---|---|---|---|
| REMARK | 3 | PROTEIN ATOMS | : | 84 |
| REMARK | 3 | NUCLEIC ACID ATOMS | : | 0 |
| REMARK | 3 | HETEROGEN ATOMS | : | 0 |
| REMARK | 3 | SOLVENT ATOMS | : | 1 |

| REMARK | 3 | | | | |
|---|---|---|---|---|---|
| REMARK | 3 | B VALUES. | | | |
| REMARK | 3 | FROM WILSON PLOT | (A**2) | : | NULL |
| REMARK | 3 | MEAN B VALUE | (OVERALL, A**2) | : | 40.50 |

| REMARK | 3 | OVERALL ANISOTROPIC B VALUE. | | | |
|---|---|---|---|---|---|
| REMARK | 3 | B11 | (A**2) | : | NULL |
| REMARK | 3 | B22 | (A**2) | : | NULL |
| REMARK | 3 | B33 | (A**2) | : | NULL |
| REMARK | 3 | B12 | (A**2) | : | NULL |
| REMARK | 3 | B13 | (A**2) | : | NULL |
| REMARK | 3 | B23 | (A**2) | : | NULL |

| REMARK | 3 | | | | |
|---|---|---|---|---|---|
| REMARK | 3 | ESTIMATED OVERALL COORDINATE ERROR. | | | |
| REMARK | 3 | ESU BASED ON R VALUE | (A) | : | 0.252 |
| REMARK | 3 | ESU BASED ON FREE R VALUE | (A) | : | 0.220 |
| REMARK | 3 | ESU BASED ON MAXIMUM LIKELIHOOD | (A) | : | 0.107 |
| REMARK | 3 | ESU FOR B VALUES BASED ON MAXIMUM LIKELIHOOD | (A**2) | : | 4.615 |

| REMARK | 3 | | | | | | | | |
|---|---|---|---|---|---|---|---|---|---|
| REMARK | 3 | CORRELATION COEFFICIENTS. | | | | | | | |
| REMARK | 3 | CORRELATION COEFFICIENT FO-FC : 0.916 | | | | | | | |
| REMARK | 3 | CORRELATION COEFFICIENT FO-FC FREE : 0.937 | | | | | | | |
| REMARK | 3 | | | | | | | | |
| REMARK | 3 | RMS DEVIATIONS FROM IDEAL VALUES | | | COUNT | | RMS | | WEIGHT |
| REMARK | 3 | BOND LENGTHS REFINED ATOMS | (A) | : | 83 | ; | 0.022 | ; | 0.023 |
| REMARK | 3 | BOND LENGTHS OTHERS | (A) | : | 54 | ; | 0.001 | ; | 0.020 |
| REMARK | 3 | BOND ANGLES REFINED ATOMS | (DEGREES) | : | 111 | ; | 1.718 | ; | 2.066 |
| REMARK | 3 | BOND ANGLES OTHERS | (DEGREES) | : | 138 | ; | 0.731 | ; | 3.000 |
| REMARK | 3 | TORSION ANGLES, PERIOD 1 | (DEGREES) | : | 10 | ; | 6.020 | ; | 5.000 |
| REMARK | 3 | TORSION ANGLES, PERIOD 2 | (DEGREES) | : | 2 | ; | 68.399 | ; | 30.000 |
| REMARK | 3 | TORSION ANGLES, PERIOD 3 | (DEGREES | : | 19 | ; | 21.339 | ; | 15.000 |
| REMARK | 3 | TORSION ANGLES, PERIOD 4 | (DEGREES) | : | NULL | ; | NULL | ; | NULL |
| REMARK | 3 | CHIRAL-CENTER RESTRAINTS | (A**3) | : | 16 | ; | 0.115 | ; | 0.200 |
| REMARK | 3 | GENERAL PLANES REFINED ATOMS | (A) | : | 82 | ; | 0.005 | ; | 0.020 |
| REMARK | 3 | GENERAL PLANES OTHERS | (A) | : | 10 | ; | 0.000 | ; | 0.020 |
| REMARK | 3 | NON-BONDED CONTACTS REFINED ATOMS | (A) | : | NULL | ; | NULL | ; | NULL |
| REMARK | 3 | NON-BONDED CONTACTS OTHERS | (A) | : | NULL | ; | NULL | ; | NULL |
| REMARK | 3 | NON-BONDED TORSION REFINED ATOMS | (A) | : | NULL | ; | NULL | ; | NULL |
| REMARK | 3 | NON-BONDED TORSION OTHERS | (A) | : | NULL | ; | NULL | ; | NULL |
| REMARK | 3 | H-BOND (X...Y) REFINED ATOMS | (A) | : | NULL | ; | NULL | ; | NULL |
| REMARK | 3 | H-BOND (X...Y) OTHERS | (A) | : | NULL | ; | NULL | ; | NULL |
| REMARK | 3 | POTENTIAL METAL-ION REFINED ATOMS | (A) | : | NULL | ; | NULL | ; | NULL |
| REMARK | 3 | POTENTIAL METAL-ION OTHERS | (A) | : | NULL | ; | NULL | ; | NULL |
| REMARK | 3 | SYMMETRY VDW REFINED ATOMS | (A) | : | NULL | ; | NULL | ; | NULL |
| REMARK | 3 | SYMMETRY VDW OTHERS | (A) | : | NULL | ; | NULL | ; | NULL |
| REMARK | 3 | SYMMETRY H-BOND REFINED ATOMS | (A) | : | NULL | ; | NULL | ; | NULL |
| REMARK | 3 | SYMMETRY H-BOND OTHERS | (A) | : | NULL | ; | NULL | ; | NULL |
| REMARK | 3 | SYMMETRY METAL-ION REFINED ATOMS | (A) | : | NULL | ; | NULL | ; | NULL |
| REMARK | 3 | SYMMETRY METAL-ION OTHERS | (A) | : | NULL | ; | NULL | ; | NULL |
| REMARK | 3 | | | | | | | | |
| REMARK | 3 | ISOTROPIC THERMAL FACTOR RESTRAINTS. | | | COUNT | | RMS | | WEIGHT |
| REMARK | 3 | MAIN-CHAIN BOND REFINED ATOMS | (A**2) | : | 54 | ; | 2.984 | ; | 2.000 |
| REMARK | 3 | MAIN-CHAIN BOND OTHER ATOMS | (A**2) | : | 22 | ; | 0.890 | ; | 2.000 |
| REMARK | 3 | MAIN-CHAIN ANGLE REFINED ATOMS | (A**2) | : | 89 | ; | 4.638 | ; | 3.000 |
| REMARK | 3 | SIDE-CHAIN BOND REFINED ATOMS | (A**2) | : | 29 | ; | 3.534 | ; | 2.000 |
| REMARK | 3 | SIDE-CHAIN ANGLE REFINED ATOMS | (A**2) | : | 22 | ; | 5.685 | ; | 3.000 |
| REMARK | 3 | | | | | | | | |
| REMARK | 3 | ANISOTROPIC THERMAL FACTOR RESTRAINTS. | | | COUNT | | RMS | | WEIGHT |
| REMARK | 3 | RIGID-BOND RESTRAINTS | (A**2) | : | NULL | ; | NULL | ; | NULL |
| REMARK | 3 | SPHERICITY; FREE ATOMS | (A**2) | : | NULL | ; | NULL | ; | NULL |
| REMARK | 3 | SPHERICITY; BONDED ATOMS | (A**2) | : | NULL | ; | NULL | ; | NULL |

| REMARK | 3 | | |
|---|---|---|---|
| REMARK | 3 | NCS RESTRAINTS STATISTICS | |
| REMARK | 3 | NUMBER OF DIFFERENT NCS GROUPS : NULL | |
| REMARK | 3 | | |
| REMARK | 3 | TLS DETAILS | |
| REMARK | 3 | NUMBER OF TLS GROUPS : NULL | |
| REMARK | 3 | | |
| REMARK | 3 | BULK SOLVENT MODELLING. | |
| REMARK | 3 | METHOD USED : MASK | |
| REMARK | 3 | PARAMETERS FOR MASK CALCULATION | |
| REMARK | 3 | VDW PROBE RADIUS | : 1.40 |
| REMARK | 3 | ION PROBE RADIUS | : 0.80 |
| REMARK | 3 | SHRINKAGE RADIUS | : 0.80 |

| REMARK | 3 | | | | |
|---|---|---|---|---|---|
| REMARK | 3 | OTHER REFINEMENT REMARKS: HYDROGENS HAVE BEEN ADDED IN THE RIDING | | | |
| REMARK | 3 | POSITIONS | | | |
| REMARK | 4 | | | | |
| REMARK | 4 | 3SGO COMPLIES WITH FORMAT V. 3.30, 13-JUL-11 | | | |
| REMARK | 100 | | | | |
| REMARK | 100 | THIS ENTRY HAS BEEN PROCESSED BY RCSB ON 24-JUN-11. | | | |
| REMARK | 100 | THE RCSB ID CODE IS RCSB066179. | | | |
| REMARK | 200 | | | | |
| REMARK | 200 | EXPERIMENTAL DETAILS | | | |
| REMARK | 200 | EXPERIMENT TYPE | | : | X-RAY DIFFRACTION |
| REMARK | 200 | DATE OF DATA COLLECTION | | : | 01-MAR-09 |
| REMARK | 200 | TEMPERATURE | (KELVIN) | : | 100 |
| REMARK | 200 | PH | | : | 6.5 |
| REMARK | 200 | NUMBER OF CRYSTALS USED | | : | 1 |
| REMARK | 200 | | | | |
| REMARK | 200 | SYNCHROTRON | (Y/N) | : | Y |
| REMARK | 200 | RADIATION SOURCE | | : | APS |
| REMARK | 200 | BEAMLINE | | : | 24-ID-E |
| REMARK | 200 | X-RAY GENERATOR MODEL | | : | NULL |
| REMARK | 200 | MONOCHROMATIC OR LAUE | (M/L) | : | M |
| REMARK | 200 | WAVELENGTH OR RANGE | (A) | : | 0.97918 |
| REMARK | 200 | MONOCHROMATOR | | : | NULL |
| REMARK | 200 | OPTICS | | : | NULL |
| REMARK | 200 | | | | |
| REMARK | 200 | DETECTOR TYPE | | : | CCD |
| REMARK | 200 | DETECTOR MANUFACTURER | | : | ADSC QUANTUM 315 |
| REMARK | 200 | INTENSITY-INTEGRATION SOFTWARE | | : | DENZO |
| REMARK | 200 | DATA SCALING SOFTWARE | | : | SCALEPACK |
| REMARK | 200 | | | | |
| REMARK | 200 | NUMBER OF UNIQUE REFLECTIONS | | : | 973 |
| REMARK | 200 | RESOLUTION RANGE HIGH | (A) | : | 2.550 |
| REMARK | 200 | RESOLUTION RANGE LOW | (A) | : | 50.000 |
| REMARK | 200 | REJECTION CRITERIA | (SIGMA(I)) | : | NULL |
| REMARK | 200 | | | | |
| REMARK | 200 | OVERALL. | | | |
| REMARK | 200 | COMPLETENESS FOR RANGE | (%) | : | 92.0 |
| REMARK | 200 | DATA REDUNDANCY | | : | 7.900 |
| REMARK | 200 | R MERGE | (I) | : | 0.07500 |
| REMARK | 200 | R SYM | (I) | : | NULL |
| REMARK | 200 | <I/SIGMA(I)> FOR THE DATA SET | | : | 15.5000 |

| REMARK | 200 | | | | |
|---|---|---|---|---|---|
| REMARK | 200 | IN THE HIGHEST RESOLUTION SHELL. | | | |
| REMARK | 200 | HIGHEST RESOLUTION SHELL, RANGE HIGH | (A) | : | 2.55 |
| REMARK | 200 | HIGHEST RESOLUTION SHELL, RANGE LOW | (A) | : | 2.64 |
| REMARK | 200 | COMPLETENESS FOR SHELL | (%) | : | 98.0 |
| REMARK | 200 | DATA REDUNDANCY IN SHELL | | : | 8.60 |
| REMARK | 200 | R MERGE FOR SHELL | (I) | : | 0.60000 |
| REMARK | 200 | R SYM FOR SHELL | (I) | : | NULL |
| REMARK | 200 | <I/SIGMA(I)>FOR SHELL | | : | NULL |

| REMARK | 200 | | |
|---|---|---|---|
| REMARK | 200 | DIFFRACTION PROTOCOL: SINGLE WAVELENGTH | |
| REMARK | 200 | METHOD USED TO DETERMINE THE STRUCTURE: MOLECULAR REPLACEMENT | |
| REMARK | 200 | SOFTWARE USED: PHASER | |
| REMARK | 200 | STARTING MODEL: NULL | |
| REMARK | 200 | | |
| REMARK | 200 | REMARK: NULL | |
| REMARK | 280 | | |
| REMARK | 280 | CRYSTAL | |
| REMARK | 280 | SOLVENT CONTENT, VS (%) : NULL | |
| REMARK | 280 | MATTHEWS COEFFICIENT, VM (ANGSTROMS**3/DA) : NULL | |
| REMARK | 280 | | |
| REMARK | 280 | CRYSTALLIZATION CONDITIONS: 0.1M BIS-TRIS PH 6.5, 45% MPD, 0.2M | |
| REMARK | 280 | AMMONIUM ACETATE, VAPOR DIFFUSION, HANGING DROP, TEMPERATURE 298K | |
| REMARK | 290 | | |
| REMARK | 290 | CRYSTALLOGRAPHIC SYMMETRY | |
| REMARK | 290 | SYMMETRY OPERATORS FOR SPACE GROUP: I 41 3 2 | |
| REMARK | 290 | | |
| REMARK | 290 | SYMOP | SYMMETRY |
| REMARK | 290 | NNNMMM | OPERATOR |
| REMARK | 290 | 1555 | X, Y, Z |
| REMARK | 290 | 2555 | −X+1/2, −Y, Z+1/2 |
| REMARK | 290 | 3555 | −X, Y+1/2, −Z+1/2 |
| REMARK | 290 | 4555 | X+1/2, −Y+1/2, −Z |
| REMARK | 290 | 5555 | Z, X, Y |
| REMARK | 290 | 6555 | Z+1/2, −X+1/2, −Y |
| REMARK | 290 | 7555 | −Z+1/2, −X, Y+1/2 |
| REMARK | 290 | 8555 | −Z, X+1/2, −Y+1/2 |
| REMARK | 290 | 9555 | Y, Z, X |
| REMARK | 290 | 10555 | −Y, Z+1/2, −X+1/2 |
| REMARK | 290 | 11555 | Y+1/2, −Z+1/2, −X |
| REMARK | 290 | 12555 | −Y+1/2, −Z, X+1/2 |
| REMARK | 290 | 13555 | Y+3/4, X+1/4, −Z+1/4 |
| REMARK | 290 | 14555 | −Y+3/4, −X+3/4, −Z+3/4 |
| REMARK | 290 | 15555 | Y+1/4, −X+1/4, Z+3/4 |
| REMARK | 290 | 16555 | −Y+1/4, X+3/4, Z+1/4 |
| REMARK | 290 | 17555 | X+3/4, Z+1/4, −Y+1/4 |
| REMARK | 290 | 18555 | −X+1/4, Z+3/4, Y+1/4 |
| REMARK | 290 | 19555 | −X+3/4, −Z+3/4, −Y+3/4 |
| REMARK | 290 | 20555 | X+1/4, −Z+1/4, Y+3/4 |
| REMARK | 290 | 21555 | Z+3/4, Y+1/4, −X+1/4 |
| REMARK | 290 | 22555 | Z+1/4, −Y+1/4, X+3/4 |
| REMARK | 290 | 23555 | −Z+1/4, Y+3/4, X+1/4 |
| REMARK | 290 | 24555 | −Z+3/4, −Y+3/4, −X+3/4 |
| REMARK | 290 | 25555 | X+1/2, Y+1/2, Z+1/2 |
| REMARK | 290 | 26555 | −X, −Y+1/2, Z |
| REMARK | 290 | 27555 | −X+1/2, Y, −Z |
| REMARK | 290 | 28555 | X, −Y, −Z+1/2 |
| REMARK | 290 | 29555 | Z+1/2, X+1/2, Y+1/2 |
| REMARK | 290 | 30555 | Z, −X, −Y+1/2 |
| REMARK | 290 | 31555 | −Z, −X+1/2, Y |
| REMARK | 290 | 32555 | −Z+1/2, X, −Y |
| REMARK | 290 | 33555 | Y+1/2, Z+1/2, X+1/2 |
| REMARK | 290 | 34555 | −Y+1/2, Z, −X |
| REMARK | 290 | 35555 | Y, −Z, −X+1/2 |
| REMARK | 290 | 36555 | −Y, −Z+1/2, X |
| REMARK | 290 | 37555 | Y+1/4, X+3/4, −Z+3/4 |
| REMARK | 290 | 38555 | −Y+1/4, −X+1/4, −Z+1/4 |
| REMARK | 290 | 39555 | Y+3/4, −X+3/4, Z+1/4 |
| REMARK | 290 | 40555 | −Y+3/4, X+1/4, Z+3/4 |
| REMARK | 290 | 41555 | X+1/4, Z+3/4, −Y+3/4 |
| REMARK | 290 | 42555 | −X+3/4, Z+1/4, Y+3/4 |
| REMARK | 290 | 43555 | −X+1/4, −Z+1/4, −Y+1/4 |
| REMARK | 290 | 44555 | X+3/4, −Z+3/4, Y+1/4 |
| REMARK | 290 | 45555 | Z+1/4, Y+3/4, −X+3/4 |
| REMARK | 290 | 46555 | Z+3/4, −Y+3/4, X+1/4 |
| REMARK | 290 | 47555 | −Z+3/4, Y+1/4, X+3/4 |
| REMARK | 290 | 48555 | −Z+1/4, −Y+1/4, −X+1/4 |

| REMARK | 290 | | | | | | |
|---|---|---|---|---|---|---|---|
| REMARK | 290 | WHERE NNN -> OPERATOR NUMBER | | | | | |
| REMARK | 290 | MMM -> TRANSLATION VECTOR | | | | | |
| REMARK | 290 | | | | | | |
| REMARK | 290 | CRYSTALLOGRAPHIC SYMMETRY TRANSFORMATIONS | | | | | |
| REMARK | 290 | THE FOLLOWING TRANSFORMATIONS OPERATE ON THE ATOM/HETATM | | | | | |
| REMARK | 290 | RECORDS IN THIS ENTRY TO PRODUCE CRYSTALLOGRAPHICALLY | | | | | |
| REMARK | 290 | RELATED MOLECULES. | | | | | |
| REMARK | 290 | SMTRY1 | 1 | 1.000000 | 0.000000 | 0.000000 | 0.00000 |
| REMARK | 290 | SMTRY2 | 1 | 0.000000 | 1.000000 | 0.000000 | 0.00000 |
| REMARK | 290 | SMTRY3 | 1 | 0.000000 | 0.000000 | 1.000000 | 0.00000 |
| REMARK | 290 | SMTRY1 | 2 | −1.000000 | 0.000000 | 0.000000 | 34.64200 |
| REMARK | 290 | SMTRY2 | 2 | 0.000000 | −1.000000 | 0.000000 | 0.00000 |
| REMARK | 290 | SMTRY3 | 2 | 0.000000 | 0.000000 | 1.000000 | 34.64200 |
| REMARK | 290 | SMTRY1 | 3 | −1.000000 | 0.000000 | 0.000000 | 0.00000 |
| REMARK | 290 | SMTRY2 | 3 | 0.000000 | 1.000000 | 0.000000 | 34.64200 |
| REMARK | 290 | SMTRY3 | 3 | 0.000000 | 0.000000 | −1.000000 | 34.64200 |
| REMARK | 290 | SMTRY1 | 4 | 1.000000 | 0.000000 | 0.000000 | 34.64200 |
| REMARK | 290 | SMTRY2 | 4 | 0.000000 | −1.000000 | 0.000000 | 34.64200 |
| REMARK | 290 | SMTRY3 | 4 | 0.000000 | 0.000000 | −1.000000 | 0.00000 |
| REMARK | 290 | SMTRY1 | 5 | 0.000000 | 0.000000 | 1.000000 | 0.00000 |
| REMARK | 290 | SMTRY2 | 5 | 1.000000 | 0.000000 | 0.000000 | 0.00000 |
| REMARK | 290 | SMTRY3 | 5 | 0.000000 | 1.000000 | 0.000000 | 0.00000 |
| REMARK | 290 | SMTRY1 | 6 | 0.000000 | 0.000000 | 1.000000 | 34.64200 |
| REMARK | 290 | SMTRY2 | 6 | −1.000000 | 0.000000 | 0.000000 | 34.64200 |
| REMARK | 290 | SMTRY3 | 6 | 0.000000 | −1.000000 | 0.000000 | 0.00000 |
| REMARK | 290 | SMTRY1 | 7 | 0.000000 | 0.000000 | −1.000000 | 34.64200 |
| REMARK | 290 | SMTRY2 | 7 | −1.000000 | 0.000000 | 0.000000 | 0.00000 |
| REMARK | 290 | SMTRY3 | 7 | 0.000000 | 1.000000 | 0.000000 | 34.64200 |
| REMARK | 290 | SMTRY1 | 8 | 0.000000 | 0.000000 | −1.000000 | 0.00000 |
| REMARK | 290 | SMTRY2 | 8 | 1.000000 | 0.000000 | 0.000000 | 34.64200 |
| REMARK | 290 | SMTRY3 | 8 | 0.000000 | −1.000000 | 0.000000 | 34.64200 |
| REMARK | 290 | SMTRY1 | 9 | 0.000000 | 1.000000 | 0.000000 | 0.00000 |
| REMARK | 290 | SMTRY2 | 9 | 0.000000 | 0.000000 | 1.000000 | 0.00000 |
| REMARK | 290 | SMTRY3 | 9 | 1.000000 | 0.000000 | 0.000000 | 0.00000 |
| REMARK | 290 | SMTRY1 | 10 | 0.000000 | −1.000000 | 0.000000 | 0.00000 |
| REMARK | 290 | SMTRY2 | 10 | 0.000000 | 0.000000 | 1.000000 | 34.64200 |
| REMARK | 290 | SMTRY3 | 10 | −1.000000 | 0.000000 | 0.000000 | 34.64200 |
| REMARK | 290 | SMTRY1 | 11 | 0.000000 | 1.000000 | 0.000000 | 34.64200 |
| REMARK | 290 | SMTRY2 | 11 | 0.000000 | 0.000000 | −1.000000 | 34.64200 |
| REMARK | 290 | SMTRY3 | 11 | −1.000000 | 0.000000 | 0.000000 | 0.00000 |
| REMARK | 290 | SMTRY1 | 12 | 0.000000 | −1.000000 | 0.000000 | 34.64200 |
| REMARK | 290 | SMTRY2 | 12 | 0.000000 | 0.000000 | −1.000000 | 0.00000 |
| REMARK | 290 | SMTRY3 | 12 | 1.000000 | 0.000000 | 0.000000 | 34.64200 |
| REMARK | 290 | SMTRY1 | 13 | 0.000000 | 1.000000 | 0.000000 | 51.96300 |
| REMARK | 290 | SMTRY2 | 13 | 1.000000 | 0.000000 | 0.000000 | 17.32100 |
| REMARK | 290 | SMTRY3 | 13 | 0.000000 | 0.000000 | −1.000000 | 17.32100 |
| REMARK | 290 | SMTRY1 | 14 | 0.000000 | −1.000000 | 0.000000 | 51.96300 |
| REMARK | 290 | SMTRY2 | 14 | −1.000000 | 0.000000 | 0.000000 | 51.96300 |
| REMARK | 290 | SMTRY3 | 14 | 0.000000 | 0.000000 | −1.000000 | 51.96300 |
| REMARK | 290 | SMTRY1 | 15 | 0.000000 | 1.000000 | 0.000000 | 17.32100 |
| REMARK | 290 | SMTRY2 | 15 | −1.000000 | 0.000000 | 0.000000 | 17.32100 |
| REMARK | 290 | SMTRY3 | 15 | 0.000000 | 0.000000 | 1.000000 | 51.96300 |
| REMARK | 290 | SMTRY1 | 16 | 0.000000 | −1.000000 | 0.000000 | 17.32100 |
| REMARK | 290 | SMTRY2 | 16 | 1.000000 | 0.000000 | 0.000000 | 51.96300 |
| REMARK | 290 | SMTRY3 | 16 | 0.000000 | 0.000000 | 1.000000 | 17.32100 |
| REMARK | 290 | SMTRY1 | 17 | 1.000000 | 0.000000 | 0.000000 | 51.96300 |
| REMARK | 290 | SMTRY2 | 17 | 0.000000 | 0.000000 | 1.000000 | 17.32100 |
| REMARK | 290 | SMTRY3 | 17 | 0.000000 | −1.000000 | 0.000000 | 17.32100 |
| REMARK | 290 | SMTRY1 | 18 | −1.000000 | 0.000000 | 0.000000 | 17.32100 |
| REMARK | 290 | SMTRY2 | 18 | 0.000000 | 0.000000 | 1.000000 | 51.96300 |
| REMARK | 290 | SMTRY3 | 18 | 0.000000 | 1.000000 | 0.000000 | 17.32100 |
| REMARK | 290 | SMTRY1 | 19 | −1.000000 | 0.000000 | 0.000000 | 51.96300 |
| REMARK | 290 | SMTRY2 | 19 | 0.000000 | 0.000000 | −1.000000 | 51.96300 |
| REMARK | 290 | SMTRY3 | 19 | 0.000000 | −1.000000 | 0.000000 | 51.96300 |
| REMARK | 290 | SMTRY1 | 20 | 1.000000 | 0.000000 | 0.000000 | 17.32100 |
| REMARK | 290 | SMTRY2 | 20 | 0.000000 | 0.000000 | −1.000000 | 17.32100 |
| REMARK | 290 | SMTRY3 | 20 | 0.000000 | 1.000000 | 0.000000 | 51.96300 |
| REMARK | 290 | SMTRY1 | 21 | 0.000000 | 0.000000 | 1.000000 | 51.96300 |
| REMARK | 290 | SMTRY2 | 21 | 0.000000 | 1.000000 | 0.000000 | 17.32100 |
| REMARK | 290 | SMTRY3 | 21 | −1.000000 | 0.000000 | 0.000000 | 17.32100 |
| REMARK | 290 | SMTRY1 | 22 | 0.000000 | 0.000000 | 1.000000 | 17.32100 |
| REMARK | 290 | SMTRY2 | 22 | 0.000000 | −1.000000 | 0.000000 | 17.32100 |
| REMARK | 290 | SMTRY3 | 22 | 1.000000 | 0.000000 | 0.000000 | 51.96300 |
| REMARK | 290 | SMTRY1 | 23 | 0.000000 | 0.000000 | −1.000000 | 17.32100 |
| REMARK | 290 | SMTRY2 | 23 | 0.000000 | 1.000000 | 0.000000 | 51.96300 |
| REMARK | 290 | SMTRY3 | 23 | 1.000000 | 0.000000 | 0.000000 | 17.32100 |
| REMARK | 290 | SMTRY1 | 24 | 0.000000 | 0.000000 | −1.000000 | 51.96300 |
| REMARK | 290 | SMTRY2 | 24 | 0.000000 | −1.000000 | 0.000000 | 51.96300 |
| REMARK | 290 | SMTRY3 | 24 | −1.000000 | 0.000000 | 0.000000 | 51.96300 |
| REMARK | 290 | SMTRY1 | 25 | 1.000000 | 0.000000 | 0.000000 | 34.64200 |
| REMARK | 290 | SMTRY2 | 25 | 0.000000 | 1.000000 | 0.000000 | 34.64200 |
| REMARK | 290 | SMTRY3 | 25 | 0.000000 | 0.000000 | 1.000000 | 34.64200 |
| REMARK | 290 | SMTRY1 | 26 | −1.000000 | 0.000000 | 0.000000 | 0.00000 |
| REMARK | 290 | SMTRY2 | 26 | 0.000000 | −1.000000 | 0.000000 | 34.64200 |
| REMARK | 290 | SMTRY3 | 26 | 0.000000 | 0.000000 | 1.000000 | 0.00000 |
| REMARK | 290 | SMTRY1 | 27 | −1.000000 | 0.000000 | 0.000000 | 34.64200 |
| REMARK | 290 | SMTRY2 | 27 | 0.000000 | 1.000000 | 0.000000 | 0.00000 |
| REMARK | 290 | SMTRY3 | 27 | 0.000000 | 0.000000 | −1.000000 | 0.00000 |
| REMARK | 290 | SMTRY1 | 28 | 1.000000 | 0.000000 | 0.000000 | 0.00000 |
| REMARK | 290 | SMTRY2 | 28 | 0.000000 | −1.000000 | 0.000000 | 0.00000 |
| REMARK | 290 | SMTRY3 | 28 | 0.000000 | 0.000000 | −1.000000 | 34.64200 |
| REMARK | 290 | SMTRY1 | 29 | 0.000000 | 0.000000 | 1.000000 | 34.64200 |
| REMARK | 290 | SMTRY2 | 29 | 1.000000 | 0.000000 | 0.000000 | 34.64200 |
| REMARK | 290 | SMTRY3 | 29 | 0.000000 | 1.000000 | 0.000000 | 34.64200 |
| REMARK | 290 | SMTRY1 | 30 | 0.000000 | 0.000000 | 1.000000 | 0.00000 |
| REMARK | 290 | SMTRY2 | 30 | −1.000000 | 0.000000 | 0.000000 | 0.00000 |
| REMARK | 290 | SMTRY3 | 30 | 0.000000 | −1.000000 | 0.000000 | 34.64200 |
| REMARK | 290 | SMTRY1 | 31 | 0.000000 | 0.000000 | −1.000000 | 0.00000 |
| REMARK | 290 | SMTRY2 | 31 | −1.000000 | 0.000000 | 0.000000 | 34.64200 |
| REMARK | 290 | SMTRY3 | 31 | 0.000000 | 1.000000 | 0.000000 | 0.00000 |
| REMARK | 290 | SMTRY1 | 32 | 0.000000 | 0.000000 | −1.000000 | 34.64200 |
| REMARK | 290 | SMTRY2 | 32 | 1.000000 | 0.000000 | 0.000000 | 0.00000 |
| REMARK | 290 | SMTRY3 | 32 | 0.000000 | −1.000000 | 0.000000 | 0.00000 |
| REMARK | 290 | SMTRY1 | 33 | 0.000000 | 1.000000 | 0.000000 | 34.64200 |
| REMARK | 290 | SMTRY2 | 33 | 0.000000 | 0.000000 | 1.000000 | 34.64200 |
| REMARK | 290 | SMTRY3 | 33 | 1.000000 | 0.000000 | 0.000000 | 34.64200 |
| REMARK | 290 | SMTRY1 | 34 | 0.000000 | −1.000000 | 0.000000 | 34.64200 |
| REMARK | 290 | SMTRY2 | 34 | 0.000000 | 0.000000 | 1.000000 | 0.00000 |
| REMARK | 290 | SMTRY3 | 34 | −1.000000 | 0.000000 | 0.000000 | 0.00000 |
| REMARK | 290 | SMTRY1 | 35 | 0.000000 | 1.000000 | 0.000000 | 0.00000 |
| REMARK | 290 | SMTRY2 | 35 | 0.000000 | 0.000000 | −1.000000 | 0.00000 |
| REMARK | 290 | SMTRY3 | 35 | −1.000000 | 0.000000 | 0.000000 | 34.64200 |
| REMARK | 290 | SMTRY1 | 36 | 0.000000 | −1.000000 | 0.000000 | 0.00000 |
| REMARK | 290 | SMTRY2 | 36 | 0.000000 | 0.000000 | −1.000000 | 34.64200 |
| REMARK | 290 | SMTRY3 | 36 | 1.000000 | 0.000000 | 0.000000 | 0.00000 |
| REMARK | 290 | SMTRY1 | 37 | 0.000000 | 1.000000 | 0.000000 | 17.32100 |
| REMARK | 290 | SMTRY2 | 37 | 1.000000 | 0.000000 | 0.000000 | 51.96300 |
| REMARK | 290 | SMTRY3 | 37 | 0.000000 | 0.000000 | −1.000000 | 51.96300 |
| REMARK | 290 | SMTRY1 | 38 | 0.000000 | −1.000000 | 0.000000 | 17.32100 |
| REMARK | 290 | SMTRY2 | 38 | −1.000000 | 0.000000 | 0.000000 | 17.32100 |
| REMARK | 290 | SMTRY3 | 38 | 0.000000 | 0.000000 | −1.000000 | 17.32100 |
| REMARK | 290 | SMTRY1 | 39 | 0.000000 | 1.000000 | 0.000000 | 51.96300 |
| REMARK | 290 | SMTRY2 | 39 | −1.000000 | 0.000000 | 0.000000 | 51.96300 |
| REMARK | 290 | SMTRY3 | 39 | 0.000000 | 0.000000 | 1.000000 | 17.32100 |
| REMARK | 290 | SMTRY1 | 40 | 0.000000 | −1.000000 | 0.000000 | 51.96300 |
| REMARK | 290 | SMTRY2 | 40 | 1.000000 | 0.000000 | 0.000000 | 17.32100 |
| REMARK | 290 | SMTRY3 | 40 | 0.000000 | 0.000000 | 1.000000 | 51.96300 |
| REMARK | 290 | SMTRY1 | 41 | 1.000000 | 0.000000 | 0.000000 | 17.32100 |
| REMARK | 290 | SMTRY2 | 41 | 0.000000 | 0.000000 | 1.000000 | 51.96300 |
| REMARK | 290 | SMTRY3 | 41 | 0.000000 | −1.000000 | 0.000000 | 51.96300 |
| REMARK | 290 | SMTRY1 | 42 | −1.000000 | 0.000000 | 0.000000 | 51.96300 |
| REMARK | 290 | SMTRY2 | 42 | 0.000000 | 0.000000 | 1.000000 | 17.32100 |
| REMARK | 290 | SMTRY3 | 42 | 0.000000 | 1.000000 | 0.000000 | 51.96300 |
| REMARK | 290 | SMTRY1 | 43 | −1.000000 | 0.000000 | 0.000000 | 17.32100 |
| REMARK | 290 | SMTRY2 | 43 | 0.000000 | 0.000000 | −1.000000 | 17.32100 |
| REMARK | 290 | SMTRY3 | 43 | 0.000000 | −1.000000 | 0.000000 | 17.32100 |
| REMARK | 290 | SMTRY1 | 44 | 1.000000 | 0.000000 | 0.000000 | 51.96300 |
| REMARK | 290 | SMTRY2 | 44 | 0.000000 | 0.000000 | −1.000000 | 51.96300 |
| REMARK | 290 | SMTRY3 | 44 | 0.000000 | 1.000000 | 0.000000 | 17.32100 |
| REMARK | 290 | SMTRY1 | 45 | 0.000000 | 0.000000 | 1.000000 | 17.32100 |
| REMARK | 290 | SMTRY2 | 45 | 0.000000 | 1.000000 | 0.000000 | 51.96300 |
| REMARK | 290 | SMTRY3 | 45 | −1.000000 | 0.000000 | 0.000000 | 51.96300 |
| REMARK | 290 | SMTRY1 | 46 | 0.000000 | 0.000000 | 1.000000 | 51.96300 |
| REMARK | 290 | SMTRY2 | 46 | 0.000000 | −1.000000 | 0.000000 | 51.96300 |
| REMARK | 290 | SMTRY3 | 46 | 1.000000 | 0.000000 | 0.000000 | 17.32100 |
| REMARK | 290 | SMTRY1 | 47 | 0.000000 | 0.000000 | −1.000000 | 51.96300 |
| REMARK | 290 | SMTRY2 | 47 | 0.000000 | 1.000000 | 0.000000 | 17.32100 |
| REMARK | 290 | SMTRY3 | 47 | 1.000000 | 0.000000 | 0.000000 | 51.96300 |
| REMARK | 290 | SMTRY1 | 48 | 0.000000 | 0.000000 | −1.000000 | 17.32100 |
| REMARK | 290 | SMTRY2 | 48 | 0.000000 | −1.000000 | 0.000000 | 17.32100 |
| REMARK | 290 | SMTRY3 | 48 | −1.000000 | 0.000000 | 0.000000 | 17.32100 |

| REMARK | 290 | | | | | | |
|---|---|---|---|---|---|---|---|
| REMARK | 290 | REMARK: NULL | | | | | |
| REMARK | 300 | | | | | | |
| REMARK | 300 | BIOMOLECULE: 1 | | | | | |
| REMARK | 300 | SEE REMARK 350 FOR THE AUTHOR PROVIDED AND/OR PROGRAM | | | | | |
| REMARK | 300 | GENERATED ASSEMBLY INFORMATION FOR THE STRUCTURE IN | | | | | |
| REMARK | 300 | THIS ENTRY. THE REMARK MAY ALSO PROVIDE INFORMATION ON | | | | | |
| REMARK | 300 | BURIED SURFACE AREA. | | | | | |
| REMARK | 350 | | | | | | |
| REMARK | 350 | COORDINATES FOR A COMPLETE MULTIMER REPRESENTING THE KNOWN | | | | | |
| REMARK | 350 | BIOLOGICALLY SIGNIFICANT OLIGOMERIZATION STATE OF THE | | | | | |
| REMARK | 350 | MOLECULE CAN BE GENERATED BY APPLYING BIOMT TRANSFORMATIONS | | | | | |
| REMARK | 350 | GIVEN BELOW. BOTH NON-CRYSTALLOGRAPHIC AND | | | | | |
| REMARK | 350 | CRYSTALLOGRAPHIC OPERATIONS ARE GIVEN. | | | | | |
| REMARK | 350 | | | | | | |
| REMARK | 350 | BIOMOLECULE: 1 | | | | | |
| REMARK | 350 | AUTHOR DETERMINED BIOLOGICAL UNIT: HEXAMERIC | | | | | |
| REMARK | 350 | SOFTWARE DETERMINED QUATERNARY STRUCTURE: HEXAMERIC | | | | | |
| REMARK | 350 | SOFTWARE USED: PISA | | | | | |
| REMARK | 350 | TOTAL BURIED SURFACE AREA: 6120 ANGSTROM**2 | | | | | |
| REMARK | 350 | SURFACE AREA OF THE COMPLEX: 4000 ANGSTROM**2 | | | | | |
| REMARK | 350 | CHANGE IN SOLVENT FREE ENERGY: −38.0 KCAL/MOL | | | | | |
| REMARK | 350 | APPLY THE FOLLOWING TO CHAINS: A | | | | | |
| REMARK | 350 | BIOMT1 | 1 | 1.000000 | 0.000000 | 0.000000 | 0.00000 |
| REMARK | 350 | BIOMT2 | 1 | 0.000000 | 1.000000 | 0.000000 | 0.00000 |
| REMARK | 350 | BIOMT3 | 1 | 0.000000 | 0.000000 | 1.000000 | 0.00000 |
| REMARK | 350 | BIOMT1 | 2 | 0.000000 | 0.000000 | 1.000000 | 0.00000 |
| REMARK | 350 | BIOMT2 | 2 | 1.000000 | 0.000000 | 0.000000 | 0.00000 |
| REMARK | 350 | BIOMT3 | 2 | 0.000000 | 1.000000 | 0.000000 | 0.00000 |
| REMARK | 350 | BIOMT1 | 3 | 0.000000 | 1.000000 | 0.000000 | 0.00000 |
| REMARK | 350 | BIOMT2 | 3 | 0.000000 | 0.000000 | 1.000000 | 0.00000 |
| REMARK | 350 | BIOMT3 | 3 | 1.000000 | 0.000000 | 0.000000 | 0.00000 |
| REMARK | 350 | BIOMT1 | 4 | 0.000000 | −1.000000 | 0.000000 | −17.32100 |
| REMARK | 350 | BIOMT2 | 4 | −1.000000 | 0.000000 | 0.000000 | −17.32100 |
| REMARK | 350 | BIOMT3 | 4 | 0.000000 | 0.000000 | −1.000000 | −17.32100 |
| REMARK | 350 | BIOMT1 | 5 | −1.000000 | 0.000000 | 0.000000 | −17.32100 |
| REMARK | 350 | BIOMT2 | 5 | 0.000000 | 0.000000 | −1.000000 | −17.32100 |
| REMARK | 350 | BIOMT3 | 5 | 0.000000 | −1.000000 | 0.000000 | −17.32100 |
| REMARK | 350 | BIOMT1 | 6 | 0.000000 | 0.000000 | −1.000000 | −17.32100 |
| REMARK | 350 | BIOMT2 | 6 | 0.000000 | −1.000000 | 0.000000 | −17.32100 |
| REMARK | 350 | BIOMT3 | 6 | −1.000000 | 0.000000 | 0.000000 | −17.32100 |

| REMARK | 900 | | | | |
|---|---|---|---|---|---|
| REMARK | 900 | RELATED ENTRIES | | | |
| REMARK | 900 | RELATED ID: 3SGM RELATED DB: PDB | | | |
| REMARK | 900 | BROMODERIVATIVE-2 OF AMYLOID-RELATED SEGMENT OF ALPHAB- | | | |
| REMARK | 900 | CRYSTALLIN RESIDUES 90-100 | | | |
| REMARK | 900 | RELATED ID: 3SGN RELATED DB: PDB | | | |
| REMARK | 900 | BROMODERIVATIVE-8 OF AMYLOID-RELATED SEGMENT OF ALPHAB- | | | |
| REMARK | 900 | CRYSTALLIN RESIDUES 90-100 | | | |
| REMARK | 900 | RELATED ID: 3SGP RELATED DB: PDB | | | |
| REMARK | 900 | AMYLOID-RELATED SEGMENT OF ALPHAB-CRYSTALLIN RESIDUES 90- | | | |
| REMARK | 900 | 100 MUTANT V91L | | | |
| REMARK | 900 | RELATED ID: 3SGR RELATED DB: PDB | | | |
| REMARK | 900 | TANDEM REPEAT OF AMYLOID-RELATED SEGMENT OF ALPHAB- | | | |
| REMARK | 900 | CRYSTALLIN RESIDUES 90-100 MUTANT V91L | | | |
| REMARK | 900 | RELATED ID: 3SGS RELATED DB: PDB | | | |
| REMARK | 900 | AMYLOID-RELATED SEGMENT OF ALPHAB-CRYSTALLIN RESIDUES 95-100 | | | |
| DBREF | 3SGO A 1 11 UNP P02511 CRYAB_HUMAN 90 100 | | | | |
| SEQRES | 1 | A 11 LYS VAL LYS VAL LEU GLY ASP VAL ILE GLU VAL | | | |
| FORMUL | 2 | HOH * (H2 O) | | | |
| CRYST1 | 69.284 69.284 69.284 90.00 90.00 90.00 I 41 3 2 48 | | | | |
| ORIGX1 | | 1.000000 | 0.000000 | 0.000000 | 0.00000 |
| ORIGX2 | | 0.000000 | 1.000000 | 0.000000 | 0.00000 |
| ORIGX3 | | 0.000000 | 0.000000 | 1.000000 | 0.00000 |
| SCALE1 | | 0.014433 | 0.000000 | 0.000000 | 0.00000 |
| SCALE2 | | 0.000000 | 0.014433 | 0.000000 | 0.00000 |
| SCALE3 | | 0.000000 | 0.000000 | 0.014433 | 0.00000 |

| | | | | | | | | | | | |
|---|---|---|---|---|---|---|---|---|---|---|---|
| ATOM | 1 | N | LYS | A | 1 | −24.855 | −12.484 | −8.933 | 1.00 | 40.97 | N |
| ATOM | 2 | CA | LYS | A | 1 | −23.912 | −11.643 | −9.703 | 1.00 | 43.94 | C |
| ATOM | 3 | C | LYS | A | 1 | −22.501 | −12.170 | −9.494 | 1.00 | 43.48 | C |
| ATOM | 4 | O | LYS | A | 1 | −22.143 | −12.633 | −8.419 | 1.00 | 43.29 | O |
| ATOM | 5 | CB | LYS | A | 1 | −24.004 | −10.170 | −9.275 | 1.00 | 46.20 | C |
| ATOM | 6 | CG | LYS | A | 1 | −23.323 | −9.216 | −10.245 | 1.00 | 47.95 | C |
| ATOM | 7 | CD | LYS | A | 1 | −23.228 | −7.777 | −9.738 | 1.00 | 48.38 | C |
| ATOM | 8 | CE | LYS | A | 1 | −22.269 | −6.989 | −10.621 | 1.00 | 50.32 | C |
| ATOM | 9 | NZ | LYS | A | 1 | −22.314 | −5.482 | −10.441 | 1.00 | 49.89 | N |
| ATOM | 10 | N | VAL | A | 2 | −21.706 | −12.122 | −10.551 | 1.00 | 44.49 | N |
| ATOM | 11 | CA | VAL | A | 2 | −20.313 | −12.533 | −10.488 | 1.00 | 39.78 | C |
| ATOM | 12 | C | VAL | A | 2 | −19.492 | −11.322 | −9.996 | 1.00 | 36.63 | C |
| ATOM | 13 | O | VAL | A | 2 | −19.705 | −10.216 | −10.481 | 1.00 | 34.12 | O |
| ATOM | 14 | CB | VAL | A | 2 | −19.892 | −13.012 | −11.882 | 1.00 | 39.42 | C |
| ATOM | 15 | CG1 | VAL | A | 2 | −18.419 | −13.232 | −11.960 | 1.00 | 39.34 | C |
| ATOM | 16 | CG2 | VAL | A | 2 | −20.691 | −14.287 | −12.217 | 1.00 | 41.20 | C |
| ATOM | 17 | N | LYS | A | 3 | −18.581 | −11.539 | −9.044 | 1.00 | 30.64 | N |
| ATOM | 18 | CA | LYS | A | 3 | −17.680 | −10.488 | −8.541 | 1.00 | 33.52 | C |
| ATOM | 19 | C | LYS | A | 3 | −16.317 | −11.094 | −8.295 | 1.00 | 32.53 | C |
| ATOM | 20 | O | LYS | A | 3 | −16.161 | −12.313 | −8.294 | 1.00 | 33.82 | O |
| ATOM | 21 | CB | LYS | A | 3 | −18.191 | −9.897 | −7.205 | 1.00 | 33.77 | C |
| ATOM | 22 | CG | LYS | A | 3 | −19.129 | −8.717 | −7.400 | 1.00 | 36.72 | C |
| ATOM | 23 | CD | LYS | A | 3 | −18.500 | −7.382 | −6.951 | 1.00 | 37.83 | C |
| ATOM | 24 | CE | LYS | A | 3 | −18.717 | −6.351 | −8.008 | 1.00 | 36.07 | C |
| ATOM | 25 | NZ | LYS | A | 3 | −18.645 | −5.037 | −7.464 | 1.00 | 37.97 | N |
| ATOM | 26 | N | VAL | A | 4 | −15.340 | −10.240 | −8.038 | 1.00 | 30.07 | N |
| ATOM | 27 | CA | VAL | A | 4 | −13.998 | −10.700 | −7.716 | 1.00 | 27.12 | C |
| ATOM | 28 | C | VAL | A | 4 | −13.657 | −10.272 | −6.324 | 1.00 | 31.64 | C |
| ATOM | 29 | O | VAL | A | 4 | −13.886 | −9.101 | −5.939 | 1.00 | 34.94 | O |
| ATOM | 30 | CB | VAL | A | 4 | −12.957 | −10.152 | −8.689 | 1.00 | 28.13 | C |
| ATOM | 31 | CG1 | VAL | A | 4 | −11.512 | −10.401 | −8.141 | 1.00 | 25.22 | C |
| ATOM | 32 | CG2 | VAL | A | 4 | −13.178 | −10.795 | −10.117 | 1.00 | 20.64 | C |
| ATOM | 33 | N | LEU | A | 5 | −13.147 | −11.245 | −5.561 | 1.00 | 33.28 | N |
| ATOM | 34 | CA | LEU | A | 5 | −12.686 | −11.049 | −4.194 | 1.00 | 31.90 | C |
| ATOM | 35 | C | LEU | A | 5 | −11.298 | −11.650 | −4.090 | 1.00 | 33.73 | C |
| ATOM | 36 | O | LEU | A | 5 | −11.067 | −12.792 | −4.508 | 1.00 | 35.84 | O |
| ATOM | 37 | CB | LEU | A | 5 | −13.621 | −11.728 | −3.208 | 1.00 | 30.11 | C |
| ATOM | 38 | CG | LEU | A | 5 | −13.380 | −11.486 | −1.708 | 1.00 | 31.08 | C |
| ATOM | 39 | CD1 | LEU | A | 5 | −13.556 | −9.949 | −1.270 | 1.00 | 23.54 | C |
| ATOM | 40 | CD2 | LEU | A | 5 | −14.273 | −12.456 | −0.877 | 1.00 | 29.00 | C |
| ATOM | 41 | N | GLY | A | 6 | −10.361 | −10.886 | −3.547 | 1.00 | 32.12 | N |
| ATOM | 42 | CA | GLY | A | 6 | −9.010 | −11.366 | −3.487 | 1.00 | 32.33 | C |
| ATOM | 43 | C | GLY | A | 6 | −8.181 | −10.624 | −2.488 | 1.00 | 32.43 | C |
| ATOM | 44 | O | GLY | A | 6 | −8.692 | −9.795 | −1.708 | 1.00 | 32.15 | O |
| ATOM | 45 | N | ASP | A | 7 | −6.889 | −10.897 | −2.585 | 1.00 | 30.73 | N |
| ATOM | 46 | CA | ASP | A | 7 | −5.891 | −10.441 | −1.647 | 1.00 | 34.68 | C |
| ATOM | 47 | C | ASP | A | 7 | −4.720 | −9.800 | −2.364 | 1.00 | 35.46 | C |
| ATOM | 48 | O | ASP | A | 7 | −4.394 | −10.154 | −3.515 | 1.00 | 32.87 | O |
| ATOM | 49 | CB | ASP | A | 7 | −5.377 | −11.634 | −0.835 | 1.00 | 36.92 | C |
| ATOM | 50 | CG | ASP | A | 7 | −6.370 | −12.068 | 0.209 | 1.00 | 41.27 | C |
| ATOM | 51 | OD1 | ASP | A | 7 | −6.469 | −11.352 | 1.244 | 1.00 | 43.42 | O |
| ATOM | 52 | OD2 | ASP | A | 7 | −7.065 | −13.083 | −0.025 | 1.00 | 43.67 | O |
| ATOM | 53 | N | VAL | A | 8 | −4.105 | −8.858 | −1.668 | 1.00 | 33.09 | N |
| ATOM | 54 | CA | VAL | A | 8 | −2.832 | −8.269 | −2.066 | 1.00 | 38.38 | C |
| ATOM | 55 | C | VAL | A | 8 | −1.744 | −9.110 | −1.410 | 1.00 | 42.35 | C |
| ATOM | 56 | O | VAL | A | 8 | −1.751 | −9.296 | −0.205 | 1.00 | 49.84 | O |
| ATOM | 57 | CB | VAL | A | 8 | −2.788 | −6.762 | −1.632 | 1.00 | 38.67 | C |
| ATOM | 58 | CG1 | VAL | A | 8 | −1.532 | −6.084 | −2.067 | 1.00 | 40.20 | C |
| ATOM | 59 | CG2 | VAL | A | 8 | −4.012 | −6.021 | −2.246 | 1.00 | 34.26 | C |
| ATOM | 60 | N | ILE | A | 9 | −0.849 | −9.682 | −2.211 | 1.00 | 44.07 | N |
| ATOM | 61 | CA | ILE | A | 9 | 0.199 | −10.592 | −1.717 | 1.00 | 40.69 | C |
| ATOM | 62 | C | ILE | A | 9 | 1.556 | −10.087 | −2.165 | 1.00 | 38.31 | C |
| ATOM | 63 | O | ILE | A | 9 | 1.659 | −9.328 | −3.114 | 1.00 | 38.87 | O |
| ATOM | 64 | CB | ILE | A | 9 | −0.014 | −12.062 | −2.240 | 1.00 | 40.86 | C |
| ATOM | 65 | CG1 | ILE | A | 9 | −0.081 | −12.103 | −3.777 | 1.00 | 41.16 | C |
| ATOM | 66 | CG2 | ILE | A | 9 | −1.324 | −12.641 | −1.698 | 1.00 | 40.92 | C |
| ATOM | 67 | CD1 | ILE | A | 9 | 0.144 | −13.497 | −4.406 | 1.00 | 40.42 | C |
| ATOM | 68 | N | GLU | A | 10 | 2.596 | −10.541 | −1.494 | 1.00 | 45.50 | N |
| ATOM | 69 | CA | GLU | A | 10 | 3.987 | −10.290 | −1.888 | 1.00 | 51.45 | C |
| ATOM | 70 | C | GLU | A | 10 | 4.463 | −11.554 | −2.619 | 1.00 | 48.36 | C |
| ATOM | 71 | O | GLU | A | 10 | 4.295 | −12.641 | −2.107 | 1.00 | 47.13 | O |
| ATOM | 72 | CB | GLU | A | 10 | 4.821 | −10.025 | −0.614 | 1.00 | 58.42 | C |
| ATOM | 73 | CG | GLU | A | 10 | 6.202 | −9.387 | −0.804 | 1.00 | 65.71 | C |
| ATOM | 74 | CD | GLU | A | 10 | 6.174 | −7.845 | −0.818 | 1.00 | 73.16 | C |
| ATOM | 75 | OE1 | GLU | A | 10 | 6.647 | −7.233 | −1.822 | 1.00 | 77.26 | O |
| ATOM | 76 | OE2 | GLU | A | 10 | 5.693 | −7.242 | 0.176 | 1.00 | 75.10 | O |
| ATOM | 77 | N | VAL | A | 11 | 5.000 | −11.426 | −3.830 | 1.00 | 48.91 | N |
| ATOM | 78 | CA | VAL | A | 11 | 5.651 | 12.563 | −4.539 | 1.00 | 48.42 | C |
| ATOM | 79 | C | VAL | A | 11 | 7.088 | −12.195 | −4.927 | 1.00 | 50.41 | C |
| ATOM | 80 | O | VAL | A | 11 | 7.884 | −13.049 | −5.369 | 1.00 | 48.21 | O |
| ATOM | 81 | CB | VAL | A | 11 | 4.892 | −13.004 | −5.851 | 1.00 | 49.48 | C |
| ATOM | 82 | CG1 | VAL | A | 11 | 3.458 | −13.517 | −5.536 | 1.00 | 48.41 | C |
| ATOM | 83 | CG2 | VAL | A | 11 | 4.882 | −11.872 | −6.876 | 1.00 | 44.34 | C |
| ATOM | 84 | OXT | VAL | A | 11 | 7.489 | −11.024 | −4.809 | 1.00 | 49.33 | O |
| TER | 85 | VAL | | A | 11 | | | | | | |
| HETATM | 86 | O | HOH | A | 12 | −18.470 | −8.340 | −12.037 | 1.00 | 33.86 | O |

| | | | | | | | | | | | | |
|---|---|---|---|---|---|---|---|---|---|---|---|---|
| MASTER | 440 | 0 | 0 | 0 | 0 | 0 | 0 | 6 | 85 | 1 | 0 | 1 |

END

### Example II

### Abeta Cylindrin

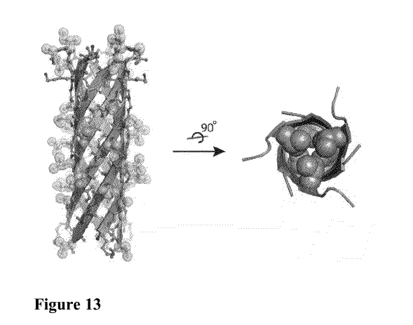

An important question is whether the ABC cylindrin is a possible model for amyloid oligomers formed by well-studied toxic proteins, such as Abeta and hIAPP. There is evidence that amyloid oligomers share common structural features. For example, studies have suggested oligomers are beta-sheet rich (38-40), and several toxic oligomers are recognized by the A11 conformational antibody (41), which also recognizes the cylindrin. A11 also recognizes alpha-hemolysin, a soluble beta barrel protein (42). Thus the cylindrin structure may represent the common structural core of amyloid oligomers. To investigate this possibility, we used the Rosetta-Profile method (18) to ask if other toxic sequences, or segments of them, are compatible with the cylindrin structure. Some of these cylindrin-forming sequences are shown in Table 7. We found that the C-terminal segment of Abeta is reasonably compatible with the cylindrin structure, and with a two-residue registration shift between pairs of anti-parallel strands, a very good fit with the cylindrin structure is obtained. (FIG. 13) This finding is in agreement with the observation of hexamers of Abeta oligomers by native mass spectrometry analysis (43).

Cylindrins of Abeta have also been generated. For example, the tandem 3 (GG) appears to dimerize immediately as it comes off of an MBP trap column, eluted at 200 mM NaCl, 20 mM Tris 7.5 (room temp) as seen by SDS-PAGE. The product, as measured by the size of the oligomer, appears to contain a mixture of dimer/monomer after Q˜400 mM NaCl, 10% glycerol, 20 mM Tris 7.5. On SEC (150 mM NaCl, 20 mM tris 7.5 room temp), it appears to be a mix of monomer and a broad peak of dimer and higher-mers. Abeta cylindrins made by a method of the invention will be tested for toxicity. It is expected that they will be cytotoxic.

### Example III

### Isolation and Characterization of a Cylindrin of SOD1

SOD1 binds copper and zinc ions and is one of three superoxide dismutases responsible for destroying free superoxide radicals in the body. This amyloid protein is involved in several pathological conditions or diseases. For example, mutations (over 150 identified to date) in this gene have been linked to familial amyotrophic lateral sclerosis. Several pieces of evidence also show that wild-type SOD1, under conditions of cellular stress, is implicated in a significant fraction of sporadic ALS cases, which represent 90% of ALS patients.

The SOD1 cylindrin-forming sequences were identified by methods as described herein, using the ABC cylindrin structure obtained in Example I as the profiled structure. Some of the cylindrin-forming segments which were identified, as well as variants thereof, are shown in Table 7.

Copies of the cylindrin-forming segments, KVKVWGSIKGL (SEQ ID NO:61) and PVKVWGSIKGL (SEQ ID NO:60), were chemically synthesized and purified by HPLC. The peptides were allowed to form aggregates as follows. It is presumed that condition 1 forms primarily amyloid fibrils, and condition 2 forms the cylindrin.

Buffer: 50 mM Tris Base

Condition 1: 37° C., shaking, overnight (produces mildly toxic species)

Condition 2: 37° C. incubation, non-agitated, overnight (produces toxic species)

The cylindrins were assayed for toxicity as follows:

Protein concentrations: 50 uM to 800 uM
Cell line: 1) Hela 2) ES derived motor neurons (GFP+)
Toxicity assay: MTT assay, and visual inspection of the GFP+ neuron morphologies
Sample incubation time: 24 hours before MTT reagents were added.

The SOD1-derived peptide KVKVWGSIKGL (SEQ ID NO:61) was crystallized and data collection was performed as follows: The peptide was crystallized via hanging-drop vapor diffusion at 18° C. The peptide was dissolved at 50 mg/mL in 50 mM Tris, then was mixed 1:1 with the reservoir solution: 0.2M Na Citrate pH 5, 13% PEG 6000. The peptide grew into needle-shaped crystals in 2-3 days. For data collection, hundreds of crystals were mounted because of their quick decay. However, two larger crystals were able to be used to collect complete data sets, by diffracting from several points along the length of the crystals. Diffraction from iodine-soaked crystals was used to obtain phases by SIRAS.

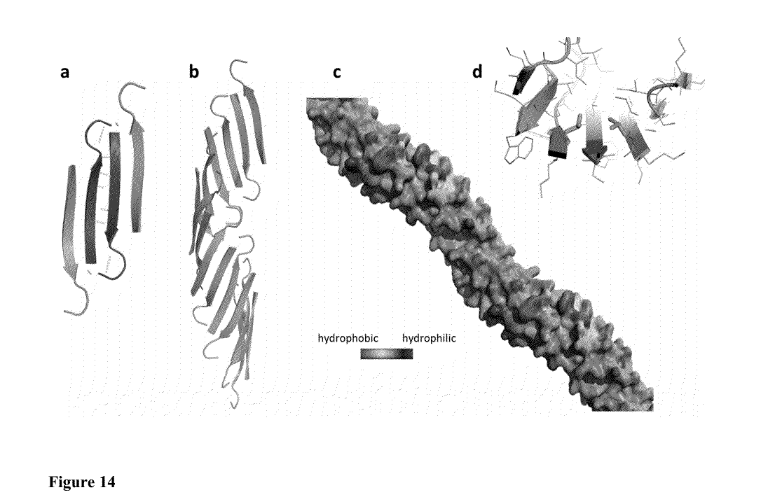

FIG. 14 shows the 3D structure of the SOD1 cylindrin.

The structure of the SOD1-derived peptide KVKVWGSIGKL (SEQ ID NO:61) is an open-ended cylindrin. Each peptide adopts a beta-strand structure, with a hydrogen-bonded turn at the C-terminus. Pairs of strands hydrogen bond in an antiparallel fashion with their C-terminal turns pointing at the N-terminus of the paired strand (red strands of panel a). These pairs of strands H-bond out-of-register with additional pairs of strands, combining to form an open-ended, curved, antiparallel beta-sheet “corkscrew.” 16 strands form one turn of the corkscrew (panel b), and hydrophobic, ‘inward’-pointing side chains mostly fill up the bore of the corkscrew (panel c). This structure and its component peptide have many features in common with the ABC cylindrin: (1) each peptide forms a beta-strand; (2) beta-strands H-bond out-of-register; (3) beta-strands form an antiparallel beta-sheet; (4) the beta-sheet is curved, with hydrophobic residues filling the inner space; (5) an essential glycine points inward, allowing packing of other side chains and thereby supporting the curvature (panel d); (6) the peptide is toxic to cultured cells; (7) the peptide additionally forms amyloid-like fibrils; and (8) the peptide amyloid-like fibrils are non-toxic to cultured cells. The biggest difference between this structure and that of the ABC cylindrin is that the ABC cylindrin is a closed oligomer (that is, of fixed size), whereas this SOD1 cylindrin is an open oligomer, and can contain any number of peptide units.

The atomic coordinates of the SOD1 cylindrin structure are shown in Table 6.

TABLE 6
Atomic coordinates of SOD1 cylindrin

| | |
|---|---|
| REMARK | --------------------- added by autoBUSTER --------------------- |

-------

| REMARK | run at | = Tue Dec 18 14:44:13 PST 2012 |
|---|---|---|
| REMARK | in | = /home/ssangwan/kv11/buster6 |
| REMARK | user | = ssangwan |
| REMARK | cmd | = refine -p refine-coot-l.pdb -m \ |
| REMARK | | kv11_2.10anom_FreeR_flag_F_P_212121.mtz -M TLSbasic - |
| autoncs | \ | |
| REMARK | | -d results |
| REMARK | Files used: | |
| REMARK | PDB | = results/pdbchk.pdb |
| REMARK | MTZ | = results/mtzchk.mtz |

| | |
|---|---|
| REMARK | output written to subdirectory = results |
| REMARK | best refinement for FP, SIGFP with R/Rfree 0.2134/0.2583 |
| REMARK | header records are copied from input PDB file (apart from REMARK |

3!)

| REMARK | --------------------- added by autoBUSTER --------------------- | |
|---|---|---|
| ------- | | |
| REMARK | --------------------- added by autoBUSTER --------------------- | |
| ------- | | |
| REMARK | run at | = Mon Dec 17 19:08:21 PST 2012 |
| REMARK | in | = /home/ssangwan/kv11/buster5 |
| REMARK | user | = ssangwan |
| REMARK | cmd | = refine -p refine-coot-0.pdb -m \ |
| REMARK | | kv11_2.10anom_FreeR_flag_F_P_212121.mtz -M TLSbasic - |
| autoncs | \ | |
| REMARK | | -d results |
| REMARK | Files used: | |
| REMARK | PDB | = results/pdbchk.pdb |
| REMARK | MTZ | = results/mtzchk.mtz |

| | |
|---|---|
| REMARK | output written to subdirectory = results |
| REMARK | best refinement for FP, SIGFP with R/Rfree 0.2096/0.2534 |
| REMARK | header records are copied from input PDB file (apart from REMARK |

3!)

| REMARK | --------------------- added by autoBUSTER --------------------- | |
|---|---|---|
| ------- | | |
| REMARK | --------------------- added by autoBUSTER --------------------- | |
| ------- | | |
| REMARK | run at | = Mon Dec 17 17:29:55 PST 2012 |
| REMARK | in | = /home/ssangwan/kv11/buster4 |
| REMARK | user | = ssangwan |
| REMARK | cmd | = refine -p refine-coot-l.pdb -m \ |
| REMARK | | kv11_2.10anom_FreeR_flag_F_P_212121.mtz -M TLSbasic - |
| autoncs | \ | |
| REMARK | | -d results |
| REMARK | Files used: | |
| REMARK | PDB | = results/pdbchk.pdb |
| REMARK | MTZ | = results/mtzchk.mtz |
| REMARK | output written to subdirectory = results | |
| REMARK | best refinement for FP, SIGFP with R/Rfree 0.2122/0.2588 | |
| REMARK | header records are copied from input PDB file (apart from REMARK | |
| 3!) | | |
| REMARK | --------------------- added by autoBUSTER --------------------- | |
| ------- | | |
| REMARK | --------------------- added by autoBUSTER --------------------- | |
| ------- | | |
| REMARK | run at | = Mon Dec 17 16:57:35 PST 2012 |
| REMARK | in | = /home/ssangwan/kv11/buster3 |
| REMARK | user | = ssangwan |
| REMARK | cmd | = refine -p refine-coot-0.pdb -m \ |
| REMARK | | ky11_2.10anom_FreeR_flag_F_P212121.mtz -M TLSbasic - |
| autoncs | \ | |
| REMARK | | -d results |
| REMARK | Files used: | |
| REMARK | PDB | = results/pdbchk.pdb |
| REMARK | MTZ | = results/mtzchk.mtz |
| REMARK | output written to subdirectory = results | |
| REMARK | best refinement for FP,SIGFP with R/Rfree 0.2182/0.2585 | |
| REMARK | header records are copied from input PDB file (apart from REMARK | |
| 3!) | | |
| REMARK | --------------------- added by autoBUSTER --------------------- | |
| ------- | | |
| REMARK | --------------------- added by autoBUSTER --------------------- | |
| ------- | | |
| REMARK | run at | = Mon Dec 17 16:02:58 PST 2012 |
| REMARK | in | = /home/ssangwan/kv11/buster2 |
| REMARK | user | = ssangwan |
| REMARK | cmd | = refine -p refine-coot-l.pdb -m \ |
| REMARK | | kv112.10anom_FreeR flag F P212121.mtz -M TLSbasic - |
| autoncs | \ | |
| REMARK | | -d results |
| REMARK | Files used: | |
| REMARK | PDB | = results/pdbchk.pdb |
| REMARK | MTZ | = results/mtzchk.mtz |
| REMARK | output written to subdirectory = results | |
| REMARK | best refinement for FP, SIGFP with R/Rfree 0.2198/0.2616 | |
| REMARK | header records are copied from input PDB file (apart from REMARK | |
| 3!) | | |
| REMARK | --------------------- added by autoBUSTER --------------------- | |
| ------- | | |
| REMARK | --------------------- added by autoBUSTER --------------------- | |
| ------- | | |
| REMARK | run at | = Fri Dec 14 15:14:08 PST 2012 |
| REMARK | in | = /home/ssangwan/kvll/buster |
| REMARK | user | = ssangwan |
| REMARK | cmd | = refine -p corkscrew005_001.pdb -m \ |
| REMARK | | kv11_2.10anom_FreeR_flag_F_P212121.mtz -M TLSbasic - |
| autoncs | \ | |
| REMARK | | -d results |
| REMARK | Files used: | |
| REMARK | PDB | = results/pdbchk.pdb |
| REMARK | MTZ | = results/mtzchk.mtz |

| | |
|---|---|
| REMARK | output written to subdirectory = results |
| REMARK | best refinement for FP, SIGFP with R/Rfree 0.2295/0.2763 |
| REMARK | header records are copied from input PDB file (apart from REMARK |
| 3!) | |
| REMARK | --------------------- added by autoBUSTER --------------------- |
| ------- | |
| REMARK | Date 2012-12-13 Time 19:42:07 PST -0800 (1355456527.79 s) |
| REMARK | PHENIX refinement |
| REMARK | ****************** INPUT FILES AND LABELS |

******************************

| REMARK | Reflections: | |
|---|---|---|
| REMARK | file name | : kv11_2.10anom.mtz |
| REMARK | labels | : [′IMEAN, SIGIMEAN′] |
| REMARK | R-free flags: | |
| REMARK | file name | : kv11_2.10anom.mtz |
| REMARK | label | : FreeRflag |
| REMARK | test_flag_value: | 0 |

| | |
|---|---|
| REMARK | Model file name(s): |
| REMARK | /home/ssangwan/kv11/corkscrew004_001-coot-0.pdb |
| REMARK | ******************** REFINEMENT SUMMARY: QUICK FACTS |

*******************

| | |
|---|---|
| REMARK | Start: r_work = 0.2249 r_free = 0.2748 bonds = 0.013 angles = |
| 1.370 | |
| REMARK | Final: r_work = 0.2205 r_free = 0.2734 bonds = 0.010 angles = |
| 1.200 | |
| REMARK | |

************************************************************************

| | |
|---|---|
| REMARK | ****************** REFINEMENT STATISTICS STEP BY STEP |

******************

| REMARK | leading digit, like 1_, means number of macro-cycle | | | | |
|---|---|---|---|---|---|
| REMARK | ------------------------------------------------------------------ | | | | |
| ------ | | | | | |
| REMARK | R-factors, x-ray target values and norm of gradient of x-ray | | | | |
| target | | | | | |
| REMARK | stage | r-work | r-free | xray_target_w | xray_target_t |
| REMARK | 0 : | 0.2857 | 0.3057 | 5.196594e + 00 | 5.233174e + 00 |
| REMARK | 1_bss: | 0.2249 | 0.2748 | 4.965655e + 00 | 5.091462e + 00 |
| REMARK | 1_ohs: | 0.2249 | 0.2748 | 4.965655e + 00 | 5.091462e + 00 |
| REMARK | 1_xyz: | 0.2216 | 0.2785 | 4.966256e + 00 | 5.104762e + 00 |
| REMARK | 1_adp: | 0.2161 | 0.2736 | 4.945399e + 00 | 5.090512e + 00 |
| REMARK | 1_occ: | 0.2227 | 0.2729 | 4.963557e + 00 | 5.097585e + 00 |
| REMARK | 2_bss: | 0.2224 | 0.2728 | 4.962266e + 00 | 5.098802e + 00 |
| REMARK | 2_ohs: | 0.2224 | 0.2728 | 4.962266e + 00 | 5.098802e + 00 |
| REMARK | 2_xyz: | 0.2177 | 0.2673 | 4.938614e + 00 | 5.083166e + 00 |
| REMARK | 2_adp: | 0.2212 | 0.2702 | 4.955699e + 00 | 5.099210e + 00 |
| REMARK | 2_occ: | 0.2211 | 0.2698 | 4.955405e + 00 | 5.098629e + 00 |
| REMARK | 3_bss: | 0.2210 | 0.2694 | 4.955121e + 00 | 5.096474e + 00 |
| REMARK | 3_ohs: | 0.2210 | 0.2694 | 4.955121e + 00 | 5.096474e + 00 |
| REMARK | 3_xyz: | 0.2210 | 0.2734 | 4.962193e + 00 | 5.102944e + 00 |
| REMARK | 3_adp: | 0.2206 | 0.2735 | 4.960861e + 00 | 5.103519e + 00 |
| REMARK | 3_occ: | 0.2205 | 0.2733 | 4.960707e + 00 | 5.103338e + 00 |
| REMARK | 3_bss: | 0.2205 | 0.2734 | 4.961056e + 00 | 5.104667e + 00 |
| REMARK | 3_ohs: | 0.2205 | 0.2734 | 4.961056e + 00 | 5.104667e + 00 |
| REMARK | | | | | |
| REMARK | stage | <pher> | fom | alpha | beta |
| REMARK | 0 : | 30.963 | 0.7438 | 0.7919 | 6214.158 |
| REMARK | 1_bss: | 27.641 | 0.7796 | 0.9315 | 4343.937 |
| REMARK | 1_ohs: | 27.641 | 0.7796 | 0.9315 | 4343.937 |
| REMARK | 1_xyz: | 28.121 | 0.7745 | 0.9245 | 4470.277 |
| REMARK | 1_adp: | 27.614 | 0.7800 | 0.9152 | 4355.166 |
| REMARK | 1_occ: | 27.581 | 0.7806 | 0.9056 | 4485.084 |
| REMARK | 2_bss: | 27.611 | 0.7803 | 0.9287 | 4513.845 |
| REMARK | 2_ohs: | 27.611 | 0.7803 | 0.9287 | 4513.845 |
| REMARK | 2_xyz: | 27.049 | 0.7861 | 0.9470 | 4322.558 |
| REMARK | 2_adp: | 27.521 | 0.7810 | 0.9488 | 4399.187 |
| REMARK | 2_occ: | 27.498 | 0.7813 | 0.9496 | 4388.105 |
| REMARK | 3_bss: | 27.428 | 0.7820 | 0.9413 | 4352.944 |
| REMARK | 3_ohs: | 27.428 | 0.7820 | 0.9413 | 4352.944 |
| REMARK | 3_xyz: | 27.596 | 0.7803 | 0.9416 | 4429.526 |
| REMARK | 3_adp: | 27.666 | 0.7795 | 0.9444 | 4425.481 |
| REMARK | 3_occ: | 27.654 | 0.7796 | 0.9448 | 4420.246 |
| REMARK | 3_bss: | 27.725 | 0.7788 | 0.9371 | 4448.117 |
| REMARK | 3_ohs: | 27.725 | 0.7788 | 0.9371 | 4448.117 |

| REMARK | | | | | | | | |
|---|---|---|---|---|---|---|---|---|
| REMARK | stage | angl | bond | chir | dihe | plan | repu | geom_target |
| REMARK | 0 : | 1.370 | 0.013 | 0.085 | 14.403 | 0.004 | 4.204 | 1.3693e−01 |
| REMARK | 1_bss: | 1.370 | 0.013 | 0.085 | 14.403 | 0.004 | 4.204 | 1.3693e−01 |
| REMARK | 1_ohs: | 1.370 | 0.013 | 0.085 | 14.403 | 0.004 | 4.204 | 1.3693e−01 |
| REMARK | 1_xyz: | 1.204 | 0.009 | 0.076 | 14.010 | 0.003 | 4.183 | 1.0037e−01 |
| REMARK | 1_adp: | 1.204 | 0.009 | 0.076 | 14.010 | 0.003 | 4.183 | 1.0037e−01 |
| REMARK | 1_occ: | 1.204 | 0.009 | 0.076 | 14.010 | 0.003 | 4.183 | 1.0037e−01 |
| REMARK | 2_bss: | 1.204 | 0.009 | 0.076 | 14.010 | 0.003 | 4.183 | 1.0037e−01 |
| REMARK | 2_ohs: | 1.204 | 0.009 | 0.076 | 14.010 | 0.003 | 4.183 | 1.0037e−01 |
| REMARK | 2_xyz: | 1.184 | 0.010 | 0.077 | 14.350 | 0.003 | 4.186 | 1.0128e−01 |
| REMARK | 2_adp: | 1.184 | 0.010 | 0.077 | 14.350 | 0.003 | 4.186 | 1.0128e−01 |
| REMARK | 2_occ: | 1.184 | 0.010 | 0.077 | 14.350 | 0.003 | 4.186 | 1.0128e−01 |
| REMARK | 3_bss: | 1.184 | 0.010 | 0.077 | 14.350 | 0.003 | 4.186 | 1.0128e−01 |
| REMARK | 3_ohs: | 1.184 | 0.010 | 0.077 | 14.350 | 0.003 | 4.186 | 1.0128e−01 |
| REMARK | 3_xyz: | 1.200 | 0.010 | 0.077 | 14.778 | 0.003 | 4.184 | 1.0323e−01 |
| REMARK | 3_adp: | 1.200 | 0.010 | 0.077 | 14.778 | 0.003 | 4.184 | 1.0323e−01 |
| REMARK | 3_occ: | 1.200 | 0.010 | 0.077 | 14.778 | 0.003 | 4.184 | 1.0323e−01 |
| REMARK | 3_bss: | 1.200 | 0.010 | 0.077 | 14.778 | 0.003 | 4.184 | 1.0323e−01 |
| REMARK | 3_ohs: | 1.200 | 0.010 | 0.077 | 14.778 | 0.003 | 4.184 | 1.0323e−01 |

| | |
|---|---|
| REMARK | ------------------------------------------------------------------ |

------

| REMARK | Maximal deviations: | | | | | | | |
|---|---|---|---|---|---|---|---|---|
| REMARK | stage | angl | bond | chir | dihe | plan | repu | \|grad\| |
| REMARK | 0 : | 12.316 | 0.148 | 0.359 | 57.322 | 0.020 | 2.343 | 3.5894e−01 |
| REMARK | 1_bss: | 12.316 | 0.148 | 0.359 | 57.322 | 0.020 | 2.343 | 3.5894e−01 |
| REMARK | 1_ohs: | 12.316 | 0.148 | 0.359 | 57.322 | 0.020 | 2.343 | 3.5894e−01 |
| REMARK | 1_xyz: | 9.794 | 0.118 | 0.325 | 47.523 | 0.008 | 2.362 | 1.1275e−01 |
| REMARK | 1_adp: | 9.794 | 0.118 | 0.325 | 47.523 | 0.008 | 2.362 | 1.1275e−01 |
| REMARK | 1_occ: | 9.794 | 0.118 | 0.325 | 47.523 | 0.008 | 2.362 | 1.1275e−01 |
| REMARK | 2_bss: | 9.794 | 0.118 | 0.325 | 47.523 | 0.008 | 2.362 | 1.1275e−01 |
| REMARK | 2_ohs: | 9.794 | 0.118 | 0.325 | 47.523 | 0.008 | 2.362 | 1.1275e−01 |
| REMARK | 2_xyz: | 10.877 | 0.128 | 0.345 | 53.980 | 0.008 | 2.474 | 9.9185e−02 |
| REMARK | 2_adp: | 10.877 | 0.128 | 0.345 | 53.980 | 0.008 | 2.474 | 9.9185e−02 |
| REMARK | 2_occ: | 10.877 | 0.128 | 0.345 | 53.980 | 0.008 | 2.474 | 9.9185e−02 |
| REMARK | 3_bss: | 10.877 | 0.128 | 0.345 | 53.980 | 0.008 | 2.474 | 9.9185e−02 |
| REMARK | 3_ohs: | 10.877 | 0.128 | 0.345 | 53.980 | 0.008 | 2.474 | 9.9185e−02 |
| REMARK | 3_xyz: | 11.358 | 0.125 | 0.336 | 56.767 | 0.008 | 2.482 | 1.2149e−01 |
| REMARK | 3_adp: | 11.358 | 0.125 | 0.336 | 56.767 | 0.008 | 2.482 | 1.2149e−01 |
| REMARK | 3_occ: | 11.358 | 0.125 | 0.336 | 56.767 | 0.008 | 2.482 | 1.2149e−01 |
| REMARK | 3_bss: | 11.358 | 0.125 | 0.336 | 56.767 | 0.008 | 2.482 | 1.2149e−01 |
| REMARK | 3_ohs: | 11.358 | 0.125 | 0.336 | 56.767 | 0.008 | 2.482 | 1.2149e−01 |

| | |
|---|---|
| REMARK | ------------------------------------------------------------------ |

------

| | |
|---|---|
| REMARK | \|-----overall-----\|---macromolecule----\|------solvent-- |

-----|

| | | | | | | | | | |
|---|---|---|---|---|---|---|---|---|---|
| REMARK | stage | b_max | b_min | b_ave | b_max | b_min | b_ave | b_max | b_min |
| 18.18 | | | | | | | | | |
| REMARK | 0 : | 36.78 | 5.61 | 12.32 | 36.78 | 5.61 | 12.10 | 30.00 | 8.26 |
| 18.18 | | | | | | | | | |
| REMARK | 1_bss: | 36.78 | 5.61 | 12.32 | 36.78 | 5.61 | 12.10 | 30.00 | 8.26 |
| 18.18 | | | | | | | | | |
| REMARK | 1_ohs: | 36.78 | 5.61 | 12.32 | 36.78 | 5.61 | 12.10 | 30.00 | 8.26 |
| 18.18 | | | | | | | | | |
| REMARK | 1_xyz: | 36.78 | 5.61 | 12.32 | 36.78 | 5.61 | 12.10 | 30.00 | 8.26 |
| 18.18 | | | | | | | | | |
| REMARK | 1_adp: | 40.21 | 3.99 | 12.28 | 40.21 | 3.99 | 12.10 | 31.36 | 8.55 |
| 17.04 | | | | | | | | | |
| REMARK | 1_occ: | 40.21 | 3.99 | 12.28 | 40.21 | 3.99 | 12.10 | 31.36 | 8.55 |
| 17.04 | | | | | | | | | |
| REMARK | 2_bss: | 40.21 | 3.99 | 12.28 | 40.21 | 3.99 | 12.10 | 31.36 | 8.55 |
| 17.04 | | | | | | | | | |
| REMARK | 2_ohs: | 40.21 | 3.99 | 12.28 | 40.21 | 3.99 | 12.10 | 31.36 | 8.55 |
| 17.04 | | | | | | | | | |
| REMARK | 2_xyz: | 40.21 | 3.99 | 12.28 | 40.21 | 3.99 | 12.10 | 31.36 | 8.55 |
| 17.04 | | | | | | | | | |
| REMARK | 2_adp: | 36.62 | 5.25 | 11.99 | 36.62 | 5.25 | 11.83 | 28.01 | 9.26 |
| 15.92 | | | | | | | | | |
| REMARK | 2_occ: | 36.62 | 5.25 | 11.99 | 36.62 | 5.25 | 11.83 | 28.01 | 9.26 |
| 15.92 | | | | | | | | | |
| REMARK | 3_bss: | 36.62 | 5.25 | 11.99 | 36.62 | 5.25 | 11.83 | 28.01 | 9.26 |
| 15.92 | | | | | | | | | |
| REMARK | 3_ohs: | 36.62 | 5.25 | 11.99 | 36.62 | 5.25 | 11.83 | 28.01 | 9.26 |
| 15.92 | | | | | | | | | |
| REMARK | 3_xyz: | 36.62 | 5.25 | 11.99 | 36.62 | 5.25 | 11.83 | 28.01 | 9.26 |
| 15.92 | | | | | | | | | |
| REMARK | 3_adp: | 36.25 | 5.23 | 12.09 | 36.25 | 5.23 | 11.94 | 28.33 | 9.39 |
| 15.97 | | | | | | | | | |
| REMARK | 3_occ: | 36.25 | 5.23 | 12.09 | 36.25 | 5.23 | 11.94 | 28.33 | 9.39 |
| 15.97 | | | | | | | | | |
| REMARK | 3_bss: | 36.25 | 5.23 | 12.09 | 36.25 | 5.23 | 11.94 | 28.33 | 9.39 |
| 15.97 | | | | | | | | | |
| REMARK | 3_ohs: | 36.25 | 5.23 | 12.09 | 36.25 | 5.23 | 11.94 | 28.33 | 9.39 |
| 15.97 | | | | | | | | | |

| REMARK | ------------------------------------------------------------------ | | | |
|---|---|---|---|---|
| ------ | | | | |
| REMARK | stage | Deviation of refined | | |
| REMARK | | model from start model | | |
| REMARK | | max | min | mean |
| REMARK | 0 : | 0.000 | 0.000 | 0.000 |
| REMARK | l_bss: | 0.000 | 0.000 | 0.000 |
| REMARK | l_ohs: | 0.000 | 0.000 | 0.000 |
| REMARK | l_xyz: | 1.737 | 0.001 | 0.045 |
| REMARK | l_adp: | 1.737 | 0.001 | 0.045 |
| REMARK | l_occ: | 1.737 | 0.001 | 0.045 |
| REMARK | 2_bss: | 1.737 | 0.001 | 0.045 |
| REMARK | 2_ohs: | 1.737 | 0.001 | 0.045 |
| REMARK | 2_xyz: | 0.746 | 0.002 | 0.055 |
| REMARK | 2_adp: | 0.746 | 0.002 | 0.055 |
| REMARK | 2_occ: | 0.746 | 0.002 | 0.055 |
| REMARK | 3_bss: | 0.746 | 0.002 | 0.055 |
| REMARK | 3_ohs: | 0.746 | 0.002 | 0.055 |
| REMARK | 3_xyz: | 2.302 | 0.005 | 0.064 |
| REMARK | 3_adp: | 2.302 | 0.005 | 0.064 |
| REMARK | 3_occ: | 2.302 | 0.005 | 0.064 |
| REMARK | 3_bss: | 2.302 | 0.005 | 0.064 |
| REMARK | 3_ohs: | 2.302 | 0.005 | 0.064 |

| REMARK | ------------------------------------------------------------------ | | |
|---|---|---|---|
| ------ | | | |
| REMARK | MODEL CONTENT. | | |
| REMARK | ELEMENT | ATOM RECORD COUNT | OCCUPANCY SUM |
| REMARK | I | 4 | 2.43 |
| REMARK | C | 466 | 466.00 |
| REMARK | O | 137 | 137.00 |
| REMARK | M | 118 | 118.00 |
| REMARK | TOTAL | 725 | 723.43 |

| REMARK | ------------------------------------------------------------------ | | |
|---|---|---|---|
| ------ | | | |
| REMARK | r_free_flags.md5.hexdigest 62fe805510beld3b379f6160905130f3 | | |
| REMARK | IF THIS FILE IS FOR PDB DEPOSITION: REMOVE ALL FROM THIS LINE UP. | | |
| REMARK | 0 : statistics at the very beginning when nothing is done yet | | |
| REMARK | 1 s: bulk solvent correction and/or (anisotropic) scaling | | |
| REMARK | 1 z: refinement of coordinates | | |
| REMARK | 1 p: refinement of ADPs (Atomic Displacement Parameters) | | |
| REMARK | 1 c: refinement of occupancies | | |
| REMARK | 3 | | |
| REMARK | 3 | REFINEMENT. | |
| REMARK | 3 | PROGRAM | : BUSTER 2.10.0 |
| REMARK | 3 | AUTHORS | : BRICOGNE, BLANC, BRANDL, FLENSBURG, KELLER, |
| REMARK | 3 | | : PACIOREK, ROVERSI, SHARFF, SMART, VONRHEIN, WOMACK; |
| REMARK | 3 | | : MATTHEWS, TEN EYCK, TRONRUD |

| REMARK | 3 | | | |
|---|---|---|---|---|
| REMARK | 3 | DATA USED IN REFINEMENT. | | |
| REMARK | 3 | RESOLUTION RANGE HIGH | (ANGSTROMS) | : 2.10 |
| REMARK | 3 | RESOLUTION RANGE LOW | (ANGSTROMS) | : 19.33 |
| REMARK | 3 | DATA CUTOFF | (SIGMA (F)) | : 0.0 |
| REMARK | 3 | COMPLETENESS FOR RANGE | (%) | : 95.84 |
| REMARK | 3 | NUMBER OF REFLECTIONS | | : 6249 |
| REMARK | 3 | | | |
| REMARK | 3 | FIT TO DATA USED IN REFINEMENT. | | |
| REMARK | 3 | CROSS-VALIDATION METHOD | | : THROUGHOUT |
| REMARK | 3 | FREE R VALUE TEST SET SELECTION | | : RANDOM |
| REMARK | 3 | R VALUE | (WORKING + TEST SET) | : 0.2156 |
| REMARK | 3 | R VALUE | (WORKING SET) | : 0.2134 |
| REMARK | 3 | FREE R VALUE | | : 0.2583 |
| REMARK | 3 | FREE R VALUE TEST SET SIZE | (%) | : 5.01 |
| REMARK | 3 | FREE R VALUE TEST SET COUNT | | : 313 |
| REMARK | 3 | ESTIMATED ERROR OF FREE R VALUE | | : NULL |

| REMARK | 3 | | | |
|---|---|---|---|---|
| REMARK | 3 | FIT IN THE HIGHEST RESOLUTION BIN. | | |
| REMARK | 3 | TOTAL NUMBER OF BINS USED | | : 5 |
| REMARK | 3 | BIN RESOLUTION RANGE HIGH | (ANGSTROMS) | : 2.10 |
| REMARK | 3 | BIN RESOLUTION RANGE LOW | (ANGSTROMS) | : 2.35 |
| REMARK | 3 | BIN COMPLETENESS (WORKING + TEST) | (%) | : 95.84 |
| REMARK | 3 | REFLECTIONS IN BIN | (WORKING + TEST SET) | : 1747 |
| REMARK | 3 | BIN R VALUE | (WORKING + TEST SET) | : 0.1755 |
| REMARK | 3 | REFLECTIONS IN BIN | (WORKING SET) | : 1660 |
| REMARK | 3 | BIN R VALUE | (WORKING SET) | : 0.1727 |
| REMARK | 3 | BIN FREE R VALUE | | : 0.2291 |
| REMARK | 3 | BIN FREE R VALUE TEST SET SIZE | (%) | : 4.98 |
| REMARK | 3 | BIN FREE R VALUE TEST SET COUNT | | : 87 |
| REMARK | 3 | ESTIMATED ERROR OF BIN FREE R VALUE | | : NULL |

| REMARK | 3 | | |
|---|---|---|---|
| REMARK | 3 | NUMBER OF NON-HYDROGEN ATOMS USED IN REFINEMENT. | |
| REMARK | 3 | PROTEIN ATOMS | : 695 |
| REMARK | 3 | NUCLEIC ACID ATOMS | : 0 |
| REMARK | 3 | HETEROGEN ATOMS | : 62 |
| REMARK | 3 | SOLVENT ATOMS | : 52 |

| REMARK | 3 | | | |
|---|---|---|---|---|
| REMARK | 3 | B VALUES. | | |
| REMARK | 3 | FROM WILSON PLOT | (A**2) | : 21.81 |
| REMARK | 3 | MEAN B VALUE | (OVERALL, A**2) | : 23.84 |

| REMARK | 3 | OVERALL ANISOTROPIC B VALUE. | | | |
|---|---|---|---|---|---|
| REMARK | 3 | B11 | (A**2) | : | −4.3738 |
| REMARK | 3 | B22 | (A**2) | : | −2.3429 |
| REMARK | 3 | B33 | (A**2) | : | 6.7168 |
| REMARK | 3 | B12 | (A**2) | : | 0.0000 |
| REMARK | 3 | B13 | (A**2) | : | 0.0000 |
| REMARK | 3 | B23 | (A**2) | : | 0.0000 |
| REMARK | 3 | | | | |

| REMARK | 3 | ESTIMATED COORDINATE ERROR. | | | |
|---|---|---|---|---|---|
| REMARK | 3 | ESD FROM LUZZATI PLOT | (A) | : | 0.244 |
| REMARK | 3 | DPI (BLOW EQ-10) BASED ON R VALUE | (A) | : | 0.235 |
| REMARK | 3 | DPI (BLOW EQ-9) BASED ON FREE R VALUE | (A) | : | 0.196 |
| REMARK | 3 | DPI (CRUICKSHANK) BASED ON R VALUE | (A) | : | 0.220 |
| REMARK | 3 | DPI (CRUICKSHANK) BASED ON FREE R VALUE | (A) | : | 0.191 |

| REMARK | 3 | | |
|---|---|---|---|
| REMARK | 3 | REFERENCES: BLOW, D. (2002) ACTA CRYST D58, 792-797 | |
| REMARK | 3 | CRUICKSHANK, D.W.J. (1999) ACTA CRYST D55, 583- | |
| 601 | | | |
| REMARK | 3 | | |
| REMARK | 3 | CORRELATION COEFFICIENTS. | |
| REMARK | 3 | CORRELATION COEFFICIENT FO-FC | : 0.9202 |
| REMARK | 3 | CORRELATION COEFFICIENT FO-FC FREE | : 0.9217 |

| REMARK | 3 | | | |
|---|---|---|---|---|
| REMARK | 3 | X-RAY WEIGHT : 6.04 | | |
| REMARK | 3 | | | |
| REMARK | 3 | GEOMETRY FUNCTION. | | |
| REMARK | 3 | RESTRAINT LIBRARIES. | | |
| REMARK | 3 | NUMBER OF LIBRARIES USED : 8 | | |
| REMARK | 3 | LIBRARY | 1 : | protgeo_eh99.dat (V1.6.4.1) 20110121 STANDARD |
| REMARK | | | | AMINO ACID DICTIONARY. BONDS AND ANGLES FROM |
| REMARK | 3 | | | ENGH AND HUBER EH99. OTHER VALUES BASED ON |
| REMARK | 3 | | | PREVIOUS TNT OR TAKEN FROM CCP4. INCLUDES |
| REMARK | 3 | | | HYDROGEN ATOMS. |
| REMARK | 3 | LIBRARY | 2 : | exoticaa.dat (V1.3.4.4) 20101005 COLLECTION |
| OF | | | | |
| REMARK | 3 | | | NON-STANDARD AMINO ACIDS, MAINLY EH91 WITHOUT |
| REMARK | 3 | | | IDEAL DISTANCE INFO |
| REMARK | 3 | LIBRARY | 3 : | nuclgeo.dat (V1.13.4.1) 20100324 |
| REMARK | 3 | LIBRARY | 4 : | bcorrel.dat (V1.15) 20080423 |
| REMARK | 3 | LIBRARY | 5 : | contact.dat (V1.15.12.5) 20110510 |
| REMARK | 3 | LIBRARY | 6 : | idealdist_contact.dat(V1.3.4.3) 20110121 |
| REMARK | 3 | | | IDEAL-DISTANCE CONTACT TERM DATA AS USED IN |
| REMARK | 3 | | | PROLSQ. VALUES USED HERE ARE BASED ON THE |
| REMARK | | | | |
| REMARK | 3 | | | 5.5 IMPLEMENTATION. |
| REMARK | 3 | LIBRARY | 7 : | restraints for GOL (GLYCEROL) from cif |
| REMARK | 3 | | | dictionary GOL.cif using refmacdict2tnt |
| revision | | | | |
| REMARK | 3 | | | 1.20.2.10; buster common-compounds v 1.0 (05 |
| May | | | | |
| REMARK | | | | 2011) |
| REMARK | 3 | LIBRARY | 8 : | assume.dat (V1.9.4.1) 20110113 |

| REMARK | 3 | | | | | | | |
|---|---|---|---|---|---|---|---|---|
| REMARK | 3 | NUMBER OF GEOMETRIC FUNCTION TERMS DEFINED : 15 | | | | | | |
| REMARK | | TERM | | COUNT | | WEIGHT | | FUNCTION. |
| REMARK | 3 | BOND LENGTHS | : | 711 | ; | 2.00 | ; | HARMONIC |
| REMARK | 3 | BOND ANGLES | : | 943 | ; | 2.00 | ; | HARMONIC |
| REMARK | 3 | TORSION ANGLES | : | 250 | ; | 2.00 | ; | SINUSOIDAL |
| REMARK | 3 | TRIGONAL CARBON PLANES | : | 8 | ; | 2.00 | ; | HARMONIC |
| REMARK | 3 | GENERAL PLANES | : | 91 | ; | 5.00 | ; | HARMONIC |
| REMARK | 3 | ISOTROPIC THERMAL FACTORS | : | 711 | ; | 20.00 | ; | HARMONIC |
| REMARK | 3 | BAD NON-BONDED CONTACTS | : | NULL | ; | NULL | ; | NULL |
| REMARK | 3 | IMPROPER TORSIONS | : | NULL | ; | NULL | ; | NULL |
| REMARK | 3 | PSEUDOROTATION ANGLES | : | NULL | ; | NULL | ; | NULL |
| REMARK | 3 | CHIRAL IMPROPER TORSION | : | 81 | ; | 5.00 | ; | SEMIHARMONIC |
| REMARK | 3 | SUM OF OCCUPANCIES | : | NULL | ; | NULL | ; | NULL |
| REMARK | 3 | UTILITY DISTANCES | : | NULL | ; | NULL | ; | NULL |
| REMARK | 3 | UTILITY ANGLES | : | NULL | ; | NULL | ; | NULL |
| REMARK | 3 | UTILITY TORSION | : | NULL | ; | NULL | ; | NULL |
| REMARK | 3 | IDEAL-DIST CONTACT TERM | : | 779 | ; | 4.00 | ; | SEMIHARMONIC |

| REMARK | 3 | | | | |
|---|---|---|---|---|---|
| REMARK | 3 | RMS DEVIATIONS FROM IDEAL VALUES. | | | |
| REMARK | 3 | BOND LENGTHS | (A) | : | 0.010 |
| REMARK | 3 | BOND ANGLES | (DEGREES) | : | 0.88 |
| REMARK | 3 | PEPTIDE OMEGA TORSION ANGLES | (DEGREES) | : | 2.41 |
| REMARK | 3 | OTHER TORSION ANGLES | (DEGREES) | : | 14.49 |

| REMARK | 3 | | |
|---|---|---|---|
| REMARK | 3 | SIMILARITY. | |
| REMARK | 3 | NCS. | |
| REMARK | 3 | NCS REPRESENTATION : RESTRAINT LSSR (-AUTONCS) | |
| REMARK | 3 | TARGET RESTRAINTS. | |
| REMARK | 3 | TARGET REPRESENTATION : NONE | |
| REMARK | 3 | TARGET STRUCTURE : NULL | |
| REMARK | 3 | | |
| REMARK | 3 | TLS DETAILS. | |
| REMARK | 3 | NUMBER OF TLS GROUPS : 0 | |
| REMARK | 3 | | |
| REMARK | 3 | REFINEMENT NOTES. | |
| REMARK | 3 | NUMBER OF REFINEMENT NOTES : 1 | |
| REMARK | 3 | NOTE 1 : IDEAL-DIST CONTACT TERM CONTACT SETUP. ALL ATOMS | |
| REMARK | 3 | HAVE CCP4 ATOM TYPE FROM LIBRARY | |
| REMARK | 3 | | |
| REMARK | 3 | OTHER REFINEMENT REMARKS: NULL | |
| REMARK | 3 | | |
| REMARK | 290 | | |
| REMARK | 290 | CRYSTALLOGRAPHIC SYMMETRY | |
| REMARK | 290 | SYMMETRY OPERATORS FOR SPACE GROUP: P 21 21 21 | |
| REMARK | 290 | | |
| REMARK | 290 | SYMOP | SYMMETRY |
| REMARK | 290 | NNNMMM | OPERATOR |
| REMARK | 290 | 1555 | X,Y,Z |
| REMARK | 290 | 2555 | 1/2-X,-Y,1/2+Z |
| REMARK | 290 | 3555 | -X,1/2+Y,1/2-Z |
| REMARK | 290 | 4555 | 1/2+X,1/2-Y,-Z |

| REMARK | 290 | | | | | | |
|---|---|---|---|---|---|---|---|
| REMARK | 290 | WHERE NNN -> OPERATOR NUMBER | | | | | |
| REMARK | 290 | MMM -> TRANSLATION VECTOR | | | | | |
| REMARK | 290 | | | | | | |
| REMARK | 290 | CRYSTALLOGRAPHIC SYMMETRY TRANSFORMATIONS | | | | | |
| REMARK | 290 | THE FOLLOWING TRANSFORMATIONS OPERATE ON THE ATOM/HETATM | | | | | |
| REMARK | 290 | RECORDS IN THIS ENTRY TO PRODUCE CRYSTALLOGRAPHICALLY | | | | | |
| REMARK | 290 | RELATED MOLECULES. | | | | | |
| REMARK | 290 | SMTRY1 | 1 | 1.000000 | 0.000000 | 0.000000 | 0.00000 |
| REMARK | 290 | SMTRY2 | 1 | 0.000000 | 1.000000 | 0.000000 | 0.00000 |
| REMARK | 290 | SMTRY3 | 1 | 0.000000 | 0.000000 | 1.000000 | 0.00000 |
| REMARK | 290 | SMTRY1 | 2 | −1.000000 | 0.000000 | 0.000000 | 16.54500 |
| REMARK | 290 | SMTRY2 | 2 | 0.000000 | −1.000000 | 0.000000 | 0.00000 |
| REMARK | 290 | SMTRY3 | 2 | 0.000000 | 0.000000 | 1.000000 | 35.71500 |
| REMARK | 290 | SMTRY1 | 3 | −1.000000 | 0.000000 | 0.000000 | 0.00000 |
| REMARK | 290 | SMTRY2 | 3 | 0.000000 | 1.000000 | 0.000000 | 22.19500 |
| REMARK | 290 | SMTRY3 | 3 | 0.000000 | 0.000000 | −1.000000 | 35.71500 |
| REMARK | 290 | SMTRY1 | 4 | 1.000000 | 0.000000 | 0.000000 | 16.54500 |
| REMARK | 290 | SMTRY2 | 4 | 0.000000 | −1.000000 | 0.000000 | 22.19500 |
| REMARK | 290 | SMTRY3 | 4 | 0.000000 | 0.000000 | −1.000000 | 0.00000 |

| | | |
|---|---|---|
| REMARK | 290 | |
| REMARK | 290 | REMARK: NULL |

| CRYST1 | 33.090 44.390 71.430 90.00 90.00 90.00 P 21 21 21 | | | | | | | | | | |
|---|---|---|---|---|---|---|---|---|---|---|---|
| ATOM | 1 | N | LYS | A | 1 | 17.310 | 20.150 | 36.981 | 1.00 | 17.80 | N |
| ATOM | 2 | CA | LYS | A | 1 | 17.621 | 20.256 | 35.558 | 1.00 | 16.53 | C |
| ATOM | 3 | CB | LYS | A | 1 | 19.141 | 20.048 | 35.311 | 1.00 | 18.61 | C |
| ATOM | 4 | CG | LYS | A | 1 | 19.569 | 19.964 | 33.825 | 1.00 | 31.25 | C |
| ATOM | 5 | CD | LYS | A | 1 | 19.855 | 21.308 | 33.184 | 1.00 | 46.17 | C |
| ATOM | 6 | CE | LYS | A | 1 | 21.312 | 21.522 | 32.865 | 1.00 | 61.12 | C |
| ATOM | 7 | NZ | LYS | A | 1 | 21.536 | 22.856 | 32.244 | 1.00 | 74.06 | N |
| ATOM | 8 | C | LYS | A | 1 | 16.789 | 19.227 | 34.768 | 1.00 | 16.72 | C |
| ATOM | 9 | O | LYS | A | 1 | 16.812 | 18.044 | 35.074 | 1.00 | 13.16 | O |
| ATOM | 10 | N | VAL | A | 2 | 16.061 | 19.695 | 33.757 | 1.00 | 12.60 | N |
| ATOM | 11 | CA | VAL | A | 2 | 15.208 | 18.843 | 32.942 | 1.00 | 11.64 | C |
| ATOM | 12 | CB | VAL | A | 2 | 13.705 | 19.193 | 33.122 | 1.00 | 14.63 | C |
| ATOM | 13 | CG1 | VAL | A | 2 | 12.841 | 18.381 | 32.169 | 1.00 | 14.58 | C |
| ATOM | 14 | CG2 | VAL | A | 2 | 13.250 | 18.966 | 34.563 | 1.00 | 13.84 | C |
| ATOM | 15 | C | VAL | A | 2 | 15.675 | 18.969 | 31.508 | 1.00 | 16.29 | C |
| ATOM | 16 | O | VAL | A | 2 | 15.510 | 20.029 | 30.888 | 1.00 | 15.15 | O |
| ATOM | 17 | N | LYS | A | 3 | 16.273 | 17.894 | 30.992 | 1.00 | 12.75 | N |
| ATOM | 18 | CA | LYS | A | 3 | 16.828 | 17.831 | 29.639 | 1.00 | 12.46 | C |
| ATOM | 19 | CB | LYS | A | 3 | 17.891 | 16.714 | 29.544 | 1.00 | 14.92 | C |
| ATOM | 20 | CG | LYS | A | 3 | 19.189 | 17.030 | 30.338 | 1.00 | 32.62 | C |
| ATOM | 21 | CD | LYS | A | 3 | 19.933 | 18.324 | 29.889 | 1.00 | 49.33 | C |
| ATOM | 22 | CE | LYS | A | 3 | 20.912 | 18.169 | 28.739 | 1.00 | 59.53 | C |
| ATOM | 23 | NZ | LYS | A | 3 | 21.446 | 19.487 | 28.297 | 1.00 | 61.46 | N |
| ATOM | 24 | C | LYS | A | 3 | 15.756 | 17.577 | 28.608 | 1.00 | 15.50 | C |
| ATOM | 25 | O | LYS | A | 3 | 14.768 | 16.918 | 28.907 | 1.00 | 12.90 | O |
| ATOM | 26 | N | VAL | A | 4 | 15.985 | 18.074 | 27.381 | 1.00 | 12.64 | N |
| ATOM | 27 | CA | VAL | A | 4 | 15.062 | 17.968 | 26.257 | 1.00 | 12.82 | C |
| ATOM | 28 | CB | VAL | A | 4 | 14.274 | 19.294 | 26.018 | 1.00 | 15.84 | C |
| ATOM | 29 | CG1 | VAL | A | 4 | 13.357 | 19.162 | 24.802 | 1.00 | 15.95 | C |
| ATOM | 30 | CG2 | VAL | A | 4 | 13.438 | 19.679 | 27.247 | 1.00 | 14.52 | C |
| ATOM | 31 | C | VAL | A | 4 | 15.890 | 17.589 | 25.050 | 1.00 | 18.77 | C |
| ATOM | 32 | O | VAL | A | 4 | 16.926 | 18.209 | 24.827 | 1.00 | 20.60 | O |
| ATOM | 33 | N | TRP | A | 5 | 15.475 | 16.556 | 24.287 | 1.00 | 14.80 | N |
| ATOM | 34 | CA | TRP | A | 5 | 16.244 | 16.139 | 23.098 | 1.00 | 15.32 | C |
| ATOM | 35 | CB | TRP | A | 5 | 17.524 | 15.321 | 23.489 | 1.00 | 14.96 | C |
| ATOM | 36 | CG | TRP | A | 5 | 17.255 | 13.878 | 23.870 | 1.00 | 16.05 | C |
| ATOM | 37 | CD1 | TRP | A | 5 | 17.374 | 12.776 | 23.061 | 1.00 | 18.65 | C |
| ATOM | 38 | NE1 | TRP | A | 5 | 16.988 | 11.640 | 23.746 | 1.00 | 17.12 | N |
| ATOM | 39 | CE2 | TRP | A | 5 | 16.607 | 11.990 | 25.013 | 1.00 | 18.59 | C |
| ATOM | 40 | CD2 | TRP | A | 5 | 16.754 | 13.397 | 25.127 | 1.00 | 15.95 | C |
| ATOM | 41 | CE3 | TRP | A | 5 | 16.408 | 14.018 | 26.349 | 1.00 | 17.10 | C |
| ATOM | 42 | CZ3 | TRP | A | 5 | 15.953 | 13.233 | 27.393 | 1.00 | 17.98 | C |
| ATOM | 43 | CH2 | TRP | A | 5 | 15.817 | 11.842 | 27.245 | 1.00 | 18.60 | C |
| ATOM | 44 | CZ2 | TRP | A | 5 | 16.184 | 11.197 | 26.081 | 1.00 | 17.66 | C |
| ATOM | 45 | C | TRP | A | 5 | 15.388 | 15.330 | 22.163 | 1.00 | 16.63 | C |
| ATOM | 46 | O | TRP | A | 5 | 14.451 | 14.646 | 22.587 | 1.00 | 16.69 | O |
| ATOM | 47 | N | GLY | A | 6 | 15.792 | 15.318 | 20.905 | 1.00 | 13.33 | N |
| ATOM | 48 | CA | GLY | A | 6 | 15.139 | 14.509 | 19.885 | 1.00 | 13.02 | C |
| ATOM | 49 | C | GLY | A | 6 | 15.107 | 15.213 | 18.551 | 1.00 | 15.83 | C |
| ATOM | 50 | O | GLY | A | 6 | 16.021 | 15.984 | 18.224 | 1.00 | 14.40 | O |
| ATOM | 51 | N | SER | A | 7 | 14.039 | 14.971 | 17.796 | 1.00 | 11.84 | N |
| ATOM | 52 | CA | SER | A | 7 | 13.852 | 15.599 | 16.486 | 1.00 | 12.33 | C |
| ATOM | 53 | CB | SER | A | 7 | 14.530 | 14.783 | 15.386 | 1.00 | 15.65 | C |
| ATOM | 54 | OG | SER | A | 7 | 13.972 | 13.486 | 15.337 | 1.00 | 22.16 | O |
| ATOM | 55 | C | SER | A | 7 | 12.376 | 15.792 | 16.219 | 1.00 | 18.70 | C |
| ATOM | 56 | O | SER | A | 7 | 11.552 | 15.097 | 16.827 | 1.00 | 19.07 | O |
| ATOM | 57 | N | ILE | A | 8 | 12.023 | 16.791 | 15.378 | 1.00 | 16.78 | N |
| ATOM | 58 | CA | ILE | A | 8 | 10.611 | 17.087 | 15.124 | 1.00 | 16.83 | C |
| ATOM | 59 | CB | ILE | A | 8 | 10.416 | 18.577 | 14.700 | 1.00 | 19.90 | C |
| ATOM | 60 | CG1 | ILE | A | 8 | 10.868 | 19.522 | 15.852 | 1.00 | 19.70 | C |
| ATOM | 61 | CD1 | ILE | A | 8 | 10.871 | 20.990 | 15.542 | 1.00 | 27.60 | C |
| ATOM | 62 | CG2 | ILE | A | 8 | 8.932 | 18.853 | 14.322 | 1.00 | 20.68 | C |
| ATOM | 63 | C | ILE | A | 8 | 10.053 | 16.094 | 14.109 | 1.00 | 19.76 | C |
| ATOM | 64 | O | ILE | A | 8 | 10.600 | 15.987 | 13.006 | 1.00 | 18.39 | O |
| ATOM | 65 | N | LYS | A | 9 | 8.971 | 15.371 | 14.475 | 1.00 | 17.28 | N |
| ATOM | 66 | CA | LYS | A | 9 | 8.339 | 14.388 | 13.569 | 1.00 | 18.06 | C |
| ATOM | 67 | CB | LYS | A | 9 | 7.115 | 13.699 | 14.219 | 1.00 | 21.27 | C |
| ATOM | 68 | CG | LYS | A | 9 | 5.910 | 14.608 | 14.487 | 1.00 | 37.81 | C |
| ATOM | 69 | CD | LYS | A | 9 | 4.653 | 13.804 | 14.824 | 1.00 | 40.18 | C |
| ATOM | 70 | CE | LYS | A | 9 | 3.525 | 14.714 | 15.235 | 1.00 | 38.18 | C |
| ATOM | 71 | NZ | LYS | A | 9 | 2.408 | 13.949 | 15.832 | 1.00 | 42.49 | N |
| ATOM | 72 | C | LYS | A | 9 | 7.938 | 15.012 | 12.223 | 1.00 | 24.43 | C |
| ATOM | 73 | O | LYS | A | 9 | 7.465 | 16.149 | 12.189 | 1.00 | 22.71 | O |
| ATOM | 74 | N | GLY | A | 10 | 8.172 | 14.272 | 11.142 | 1.00 | 23.71 | N |
| ATOM | 75 | CA | GLY | A | 10 | 7.815 | 14.690 | 9.788 | 1.00 | 23.91 | C |
| ATOM | 76 | C | GLY | A | 10 | 8.752 | 15.658 | 9.097 | 1.00 | 26.81 | C |
| ATOM | 77 | O | GLY | A | 10 | 8.516 | 15.987 | 7.935 | 1.00 | 26.48 | O |
| ATOM | 78 | N | LEU | A | 11 | 9.828 | 16.116 | 9.778 | 1.00 | 22.18 | N |
| ATOM | 79 | CA | LEU | A | 11 | 10.785 | 17.052 | 9.168 | 1.00 | 20.99 | C |
| ATOM | 80 | CB | LEU | A | 11 | 10.911 | 18.344 | 10.016 | 1.00 | 20.28 | C |
| ATOM | 81 | CG | LEU | A | 11 | 9.632 | 19.128 | 10.312 | 1.00 | 22.73 | C |
| ATOM | 82 | CD2 | LEU | A | 11 | 8.894 | 19.502 | 9.011 | 1.00 | 25.08 | C |
| ATOM | 83 | CD1 | LEU | A | 11 | 9.950 | 20.400 | 11.087 | 1.00 | 22.18 | C |
| ATOM | 84 | C | LEU | A | 11 | 12.156 | 16.423 | 8.937 | 1.00 | 22.04 | C |
| ATOM | 85 | O | LEU | A | 11 | 12.436 | 15.370 | 9.538 | 1.00 | 27.55 | O |
| ATOM | 86 | OXT | LEU | A | 11 | 12.965 | 16.999 | 8.186 | 1.00 | 25.08 | O |
| TER | 87 | | LEU | A | 11 | | | | | | |
| ATOM | 88 | N | LYS | B | 1 | 16.415 | 26.872 | 28.051 | 1.00 | 20.89 | N |
| ATOM | 89 | CA | LYS | B | 1 | 16.415 | 27.162 | 26.618 | 1.00 | 19.64 | C |
| ATOM | 90 | CB | LYS | B | 1 | 17.625 | 28.046 | 26.243 | 1.00 | 20.92 | C |
| ATOM | 91 | CG | LYS | B | 1 | 17.668 | 28.410 | 24.771 | 1.00 | 28.18 | C |
| ATOM | 92 | CD | LYS | B | 1 | 18.892 | 29.226 | 24.489 | 1.00 | 35.03 | C |
| ATOM | 93 | CE | LYS | B | 1 | 18.923 | 29.655 | 23.061 | 1.00 | 41.31 | C |
| ATOM | 94 | NZ | LYS | B | 1 | 19.876 | 30.777 | 22.842 | 1.00 | 55.41 | N |
| ATOM | 95 | C | LYS | B | 1 | 16.433 | 25.846 | 25.841 | 1.00 | 18.24 | C |
| ATOM | 96 | O | LYS | B | 1 | 17.248 | 24.998 | 26.123 | 1.00 | 14.10 | O |
| ATOM | 97 | N | VAL | B | 2 | 15.524 | 25.681 | 24.877 | 1.00 | 14.88 | N |
| ATOM | 98 | CA | VAL | B | 2 | 15.484 | 24.477 | 24.052 | 1.00 | 14.52 | C |
| ATOM | 99 | CB | VAL | B | 2 | 14.190 | 23.660 | 24.250 | 1.00 | 18.09 | C |
| ATOM | 100 | CG1 | VAL | B | 2 | 14.165 | 22.475 | 23.301 | 1.00 | 17.80 | C |
| ATOM | 101 | CG2 | VAL | B | 2 | 14.048 | 23.179 | 25.711 | 1.00 | 17.88 | C |
| ATOM | 102 | C | VAL | B | 2 | 15.702 | 24.909 | 22.594 | 1.00 | 17.39 | C |
| ATOM | 103 | O | VAL | B | 2 | 14.838 | 25.537 | 22.007 | 1.00 | 15.92 | O |
| ATOM | 104 | N | LYS | B | 3 | 16.859 | 24.579 | 22.042 | 1.00 | 14.17 | N |
| ATOM | 105 | CA | LYS | B | 3 | 17.270 | 24.948 | 20.679 | 1.00 | 14.56 | C |
| ATOM | 106 | CB | LYS | B | 3 | 18.814 | 24.875 | 20.558 | 1.00 | 15.78 | C |
| ATOM | 107 | CG | LYS | B | 3 | 19.496 | 25.973 | 21.355 | 1.00 | 30.10 | C |
| ATOM | 108 | CD | LYS | B | 3 | 20.985 | 25.735 | 21.424 | 1.00 | 43.17 | C |
| ATOM | 109 | CE | LYS | B | 3 | 21.653 | 26.780 | 22.273 | 1.00 | 53.75 | C |
| ATOM | 110 | NZ | LYS | B | 3 | 23.102 | 26.869 | 21.978 | 1.00 | 60.91 | N |
| ATOM | 111 | C | LYS | B | 3 | 16.664 | 24.023 | 19.659 | 1.00 | 18.15 | C |
| ATOM | 112 | O | LYS | B | 3 | 16.408 | 22.860 | 19.957 | 1.00 | 17.28 | O |
| ATOM | 113 | N | VAL | B | 4 | 16.438 | 24.553 | 18.444 | 1.00 | 14.78 | N |
| ATOM | 114 | CA | VAL | B | 4 | 15.865 | 23.834 | 17.301 | 1.00 | 12.68 | C |
| ATOM | 115 | CB | VAL | B | 4 | 14.367 | 24.180 | 17.074 | 1.00 | 14.50 | C |
| ATOM | 116 | CG1 | VAL | B | 4 | 13.797 | 23.370 | 15.918 | 1.00 | 12.84 | C |
| ATOM | 117 | CG2 | VAL | B | 4 | 13.548 | 23.937 | 18.342 | 1.00 | 14.37 | C |
| ATOM | 118 | C | VAL | B | 4 | 16.711 | 24.210 | 16.105 | 1.00 | 18.94 | C |
| ATOM | 119 | O | VAL | B | 4 | 16.974 | 25.396 | 15.897 | 1.00 | 20.97 | O |
| ATOM | 120 | N | TRP | B | 5 | 17.165 | 23.228 | 15.335 | 1.00 | 15.71 | N |
| ATOM | 121 | CA | TRP | B | 5 | 18.000 | 23.500 | 14.170 | 1.00 | 18.10 | C |
| ATOM | 122 | CB | TRP | B | 5 | 19.434 | 23.985 | 14.576 | 1.00 | 19.01 | C |
| ATOM | 123 | CG | TRP | B | 5 | 20.384 | 22.885 | 14.917 | 1.00 | 21.68 | C |
| ATOM | 124 | CD1 | TRP | B | 5 | 21.344 | 22.345 | 14.104 | 1.00 | 25.08 | C |
| ATOM | 125 | NE1 | TRP | B | 5 | 21.978 | 21.302 | 14.748 | 1.00 | 25.05 | N |
| ATOM | 126 | CE2 | TRP | B | 5 | 21.402 | 21.122 | 15.986 | 1.00 | 26.89 | C |
| ATOM | 127 | CD2 | TRP | B | 5 | 20.380 | 22.092 | 16.120 | 1.00 | 22.86 | C |
| ATOM | 128 | CE3 | TRP | B | 5 | 19.645 | 22.147 | 17.328 | 1.00 | 24.72 | C |
| ATOM | 129 | CZ3 | TRP | B | 5 | 19.914 | 21.215 | 18.323 | 1.00 | 26.73 | C |
| ATOM | 130 | CH2 | TRP | B | 5 | 20.931 | 20.257 | 18.158 | 1.00 | 27.69 | C |
| ATOM | 131 | CZ2 | TRP | B | 5 | 21.685 | 20.189 | 16.995 | 1.00 | 26.79 | C |
| ATOM | 132 | C | TRP | B | 5 | 18.064 | 22.279 | 13.251 | 1.00 | 19.65 | C |
| ATOM | 133 | O | TRP | B | 5 | 18.035 | 21.129 | 13.700 | 1.00 | 17.86 | O |
| ATOM | 134 | N | GLY | B | 6 | 18.183 | 22.557 | 11.974 | 1.00 | 15.79 | N |
| ATOM | 135 | CA | GLY | B | 6 | 18.334 | 21.527 | 10.963 | 1.00 | 16.05 | C |
| ATOM | 136 | C | GLY | B | 6 | 17.799 | 21.968 | 9.630 | 1.00 | 18.34 | C |
| ATOM | 137 | O | GLY | B | 6 | 17.859 | 23.156 | 9.298 | 1.00 | 16.48 | O |
| ATOM | 138 | N | SER | B | 7 | 17.252 | 21.009 | 8.873 | 1.00 | 13.88 | N |
| ATOM | 139 | CA | SER | B | 7 | 16.661 | 21.304 | 7.575 | 1.00 | 14.53 | C |
| ATOM | 140 | CB | SER | B | 7 | 17.711 | 21.246 | 6.459 | 1.00 | 17.75 | C |
| ATOM | 141 | OG | SER | B | 7 | 18.311 | 19.967 | 6.408 | 1.00 | 28.13 | O |
| ATOM | 142 | C | SER | B | 7 | 15.483 | 20.388 | 7.319 | 1.00 | 19.08 | C |
| ATOM | 143 | O | SER | B | 7 | 15.386 | 19.328 | 7.952 | 1.00 | 19.09 | O |
| ATOM | 144 | N | ILE | B | 8 | 14.523 | 20.834 | 6.490 | 1.00 | 16.19 | N |
| ATOM | 145 | CA | ILE | B | 8 | 13.316 | 20.035 | 6.214 | 1.00 | 16.28 | C |
| ATOM | 146 | CB | ILE | B | 8 | 12.097 | 20.913 | 5.816 | 1.00 | 19.06 | C |
| ATOM | 147 | CG1 | ILE | B | 8 | 11.758 | 21.921 | 6.963 | 1.00 | 19.27 | C |
| ATOM | 148 | CD1 | ILE | B | 8 | 10.636 | 22.874 | 6.682 | 1.00 | 23.02 | C |
| ATOM | 149 | CG2 | ILE | B | 8 | 10.864 | 20.023 | 5.473 | 1.00 | 18.71 | C |
| ATOM | 150 | C | ILE | B | 8 | 13.650 | 18.963 | 5.180 | 1.00 | 21.95 | C |
| ATOM | 151 | O | ILE | B | 8 | 14.102 | 19.302 | 4.082 | 1.00 | 19.80 | O |
| ATOM | 152 | N | LYS | B | 9 | 13.451 | 17.674 | 5.538 | 1.00 | 21.89 | N |
| ATOM | 153 | CA | LYS | B | 9 | 13.750 | 16.556 | 4.627 | 1.00 | 22.32 | C |
| ATOM | 154 | CB | LYS | B | 9 | 13.522 | 15.179 | 5.292 | 1.00 | 25.99 | C |
| ATOM | 155 | CG | LYS | B | 9 | 12.075 | 14.848 | 5.642 | 1.00 | 39.69 | C |
| ATOM | 156 | CD | LYS | B | 9 | 11.883 | 13.331 | 5.843 | 1.00 | 42.54 | C |
| ATOM | 157 | CE | LYS | B | 9 | 10.483 | 12.988 | 6.299 | 1.00 | 50.30 | C |
| ATOM | 158 | NZ | LYS | B | 9 | 9.459 | 13.196 | 5.237 | 1.00 | 57.23 | N |
| ATOM | 159 | C | LYS | B | 9 | 12.969 | 16.672 | 3.322 | 1.00 | 24.23 | C |
| ATOM | 160 | O | LYS | B | 9 | 11.812 | 17.082 | 3.339 | 1.00 | 22.25 | O |
| ATOM | 161 | N | GLY | B | 10 | 13.631 | 16.355 | 2.215 | 1.00 | 21.65 | N |
| ATOM | 162 | CA | GLY | B | 10 | 13.019 | 16.365 | 0.890 | 1.00 | 22.34 | C |
| ATOM | 163 | C | GLY | B | 10 | 12.966 | 17.714 | 0.192 | 1.00 | 27.69 | C |
| ATOM | 164 | O | GLY | B | 10 | 12.557 | 17.770 | −0.967 | 1.00 | 29.45 | O |
| ATOM | 165 | O | LEU | B | 11 | 15.689 | 20.126 | 0.471 | 1.00 | 19.24 | O |
| ATOM | 166 | N | LEU | B | 11 | 13.354 | 18.817 | 0.878 | 1.00 | 22.57 | N |
| ATOM | 167 | CA | LEU | B | 11 | 13.313 | 20.168 | 0.289 | 1.00 | 21.71 | C |
| ATOM | 168 | C | LEU | B | 11 | 14.696 | 20.741 | 0.018 | 1.00 | 22.05 | C |
| ATOM | 169 | CB | LEU | B | 11 | 12.469 | 21.137 | 1.172 | 1.00 | 20.72 | C |
| ATOM | 170 | CG | LEU | B | 11 | 11.024 | 20.711 | 1.464 | 1.00 | 23.19 | C |
| ATOM | 171 | CD1 | LEU | B | 11 | 10.280 | 21.781 | 2.254 | 1.00 | 21.78 | C |
| ATOM | 172 | CD2 | LEU | B | 11 | 10.245 | 20.402 | 0.164 | 1.00 | 24.03 | C |
| ATOM | 173 | OXT | LEU | B | 11 | 14.792 | 21.798 | −0.658 | 1.00 | 26.59 | O |
| TER | 174 | | LEU | B | 11 | | | | | | |
| ATOM | 175 | N | LYS | C | 1 | 9.843 | 12.976 | 17.430 | 1.00 | 18.42 | N |
| ATOM | 176 | CA | LYS | C | 1 | 9.683 | 12.706 | 18.863 | 1.00 | 16.82 | C |
| ATOM | 177 | CB | LYS | C | 1 | 9.817 | 11.190 | 19.137 | 1.00 | 18.50 | C |
| ATOM | 178 | CG | LYS | C | 1 | 9.833 | 10.782 | 20.627 | 1.00 | 23.17 | C |
| ATOM | 179 | CD | LYS | C | 1 | 8.436 | 10.657 | 21.267 | 1.00 | 34.69 | C |
| ATOM | 180 | CE | LYS | C | 1 | 8.034 | 9.234 | 21.570 | 1.00 | 47.09 | C |
| ATOM | 181 | NZ | LYS | C | 1 | 6.724 | 9.189 | 22.261 | 1.00 | 59.29 | N |
| ATOM | 182 | C | LYS | C | 1 | 10.718 | 13.519 | 19.678 | 1.00 | 17.09 | C |
| ATOM | 183 | O | LYS | C | 1 | 11.911 | 13.454 | 19.406 | 1.00 | 14.90 | O |
| ATOM | 184 | N | VAL | C | 2 | 10.246 | 14.269 | 20.669 | 1.00 | 13.13 | N |
| ATOM | 185 | CA | VAL | C | 2 | 11.103 | 15.073 | 21.522 | 1.00 | 12.70 | C |
| ATOM | 186 | CB | VAL | C | 2 | 10.845 | 16.594 | 21.355 | 1.00 | 16.32 | C |
| ATOM | 187 | CG1 | VAL | C | 2 | 11.689 | 17.394 | 22.343 | 1.00 | 14.77 | C |
| ATOM | 188 | CG2 | VAL | C | 2 | 11.129 | 17.039 | 19.914 | 1.00 | 15.68 | C |
| ATOM | 189 | C | VAL | C | 2 | 10.943 | 14.600 | 22.956 | 1.00 | 17.88 | C |
| ATOM | 190 | O | VAL | C | 2 | 9.886 | 14.808 | 23.558 | 1.00 | 17.28 | O |
| ATOM | 191 | N | LYS | C | 3 | 12.004 | 13.970 | 23.499 | 1.00 | 13.16 | N |
| ATOM | 192 | CA | LYS | C | 3 | 11.983 | 13.411 | 24.852 | 1.00 | 12.54 | C |
| ATOM | 193 | C | LYS | C | 3 | 12.303 | 14.445 | 25.882 | 1.00 | 14.97 | C |
| ATOM | 194 | O | LYS | C | 3 | 13.014 | 15.399 | 25.593 | 1.00 | 11.70 | O |
| ATOM | 195 | CB | LYS | C | 3 | 12.985 | 12.246 | 24.979 | 1.00 | 14.53 | C |
| ATOM | 196 | CG | LYS | C | 3 | 12.675 | 11.017 | 24.126 | 1.00 | 27.61 | C |
| ATOM | 197 | CD | LYS | C | 3 | 11.393 | 10.278 | 24.499 | 1.00 | 37.20 | C |
| ATOM | 198 | CE | LYS | C | 3 | 11.582 | 9.235 | 25.556 | 1.00 | 57.11 | C |
| ATOM | 199 | NZ | LYS | C | 3 | 10.421 | 8.313 | 25.624 | 1.00 | 72.28 | N |
| ATOM | 200 | N | VAL | C | 4 | 11.793 | 14.244 | 27.108 | 1.00 | 13.82 | N |
| ATOM | 201 | CA | VAL | C | 4 | 12.046 | 15.134 | 28.244 | 1.00 | 12.38 | C |
| ATOM | 202 | CB | VAL | C | 4 | 10.795 | 16.016 | 28.531 | 1.00 | 15.60 | C |
| ATOM | 203 | CG1 | VAL | C | 4 | 10.984 | 16.855 | 29.796 | 1.00 | 15.36 | C |
| ATOM | 204 | CG2 | VAL | C | 4 | 10.470 | 16.919 | 27.329 | 1.00 | 14.95 | C |
| ATOM | 205 | C | VAL | C | 4 | 12.412 | 14.254 | 29.438 | 1.00 | 16.21 | C |
| ATOM | 206 | O | VAL | C | 4 | 11.738 | 13.259 | 29.677 | 1.00 | 16.49 | O |
| ATOM | 207 | N | TRP | C | 5 | 13.469 | 14.605 | 30.188 | 1.00 | 13.09 | N |
| ATOM | 208 | CA | TRP | C | 5 | 13.851 | 13.819 | 31.370 | 1.00 | 13.09 | C |
| ATOM | 209 | CB | TRP | C | 5 | 14.633 | 12.549 | 30.946 | 1.00 | 12.24 | C |
| ATOM | 210 | CG | TRP | C | 5 | 15.168 | 11.732 | 32.095 | 1.00 | 13.28 | C |
| ATOM | 211 | CD1 | TRP | C | 5 | 14.463 | 10.886 | 32.900 | 1.00 | 16.03 | C |
| ATOM | 212 | NE1 | TRP | C | 5 | 15.311 | 10.279 | 33.806 | 1.00 | 15.64 | N |
| ATOM | 213 | CE2 | TRP | C | 5 | 16.589 | 10.722 | 33.591 | 1.00 | 15.98 | C |
| ATOM | 214 | CD2 | TRP | C | 5 | 16.533 | 11.674 | 32.547 | 1.00 | 12.77 | C |
| ATOM | 215 | CE3 | TRP | C | 5 | 17.731 | 12.265 | 32.106 | 1.00 | 14.10 | C |
| ATOM | 216 | CZ3 | TRP | C | 5 | 18.912 | 11.960 | 32.768 | 1.00 | 14.84 | C |
| ATOM | 217 | CH2 | TRP | C | 5 | 18.931 | 11.030 | 33.819 | 1.00 | 15.34 | C |
| ATOM | 218 | CZ2 | TRP | C | 5 | 17.773 | 10.419 | 34.264 | 1.00 | 15.41 | C |
| ATOM | 219 | C | TRP | C | 5 | 14.711 | 14.637 | 32.317 | 1.00 | 17.44 | C |
| ATOM | 220 | O | TRP | C | 5 | 15.641 | 15.314 | 31.876 | 1.00 | 16.05 | O |
| ATOM | 221 | N | GLY | C | 6 | 14.454 | 14.506 | 33.606 | 1.00 | 12.77 | N |
| ATOM | 222 | CA | GLY | C | 6 | 15.309 | 15.150 | 34.589 | 1.00 | 13.74 | C |
| ATOM | 223 | C | GLY | C | 6 | 14.653 | 15.221 | 35.943 | 1.00 | 18.51 | C |
| ATOM | 224 | O | GLY | C | 6 | 13.872 | 14.334 | 36.317 | 1.00 | 18.16 | O |
| ATOM | 225 | N | SER | C | 7 | 14.970 | 16.269 | 36.675 | 1.00 | 13.62 | N |
| ATOM | 226 | CA | SER | C | 7 | 14.402 | 16.487 | 38.008 | 1.00 | 14.47 | C |
| ATOM | 227 | CB | SER | C | 7 | 15.187 | 15.752 | 39.099 | 1.00 | 17.62 | C |
| ATOM | 228 | OG | SER | C | 7 | 16.530 | 16.179 | 39.151 | 1.00 | 27.15 | O |
| ATOM | 229 | C | SER | C | 7 | 14.283 | 17.968 | 38.280 | 1.00 | 19.33 | C |
| ATOM | 230 | O | SER | C | 7 | 14.986 | 18.754 | 37.645 | 1.00 | 19.33 | O |
| ATOM | 231 | N | ILE | C | 8 | 13.320 | 18.362 | 39.136 | 1.00 | 15.50 | N |
| ATOM | 232 | CA | ILE | C | 8 | 13.069 | 19.765 | 39.411 | 1.00 | 16.38 | C |
| ATOM | 233 | CB | ILE | C | 8 | 11.593 | 20.036 | 39.828 | 1.00 | 19.09 | C |
| ATOM | 234 | CG1 | ILE | C | 8 | 10.600 | 19.550 | 38.738 | 1.00 | 20.00 | C |
| ATOM | 235 | CD1 | ILE | C | 8 | 9.142 | 19.624 | 39.120 | 1.00 | 25.01 | C |
| ATOM | 236 | CG2 | ILE | C | 8 | 11.378 | 21.555 | 40.138 | 1.00 | 18.08 | C |
| ATOM | 237 | C | ILE | C | 8 | 14.073 | 20.245 | 40.463 | 1.00 | 24.21 | C |
| ATOM | 238 | O | ILE | C | 8 | 14.108 | 19.715 | 41.577 | 1.00 | 24.02 | O |
| ATOM | 239 | N | LYS | C | 9 | 14.887 | 21.244 | 40.107 | 1.00 | 22.97 | N |
| ATOM | 240 | CA | LYS | C | 9 | 15.884 | 21.834 | 41.028 | 1.00 | 23.02 | C |
| ATOM | 241 | CB | LYS | C | 9 | 16.722 | 22.939 | 40.332 | 1.00 | 25.72 | C |
| ATOM | 242 | CG | LYS | C | 9 | 15.928 | 24.173 | 39.899 | 1.00 | 41.01 | C |
| ATOM | 243 | CD | LYS | C | 9 | 16.816 | 25.369 | 39.566 | 1.00 | 52.18 | C |
| ATOM | 244 | CE | LYS | C | 9 | 16.032 | 26.491 | 38.926 | 1.00 | 55.89 | C |
| ATOM | 245 | NZ | LYS | C | 9 | 15.075 | 27.128 | 39.868 | 1.00 | 63.16 | N |
| ATOM | 246 | C | LYS | C | 9 | 15.205 | 22.391 | 42.295 | 1.00 | 25.21 | C |
| ATOM | 247 | O | LYS | C | 9 | 14.110 | 22.929 | 42.219 | 1.00 | 23.98 | O |
| ATOM | 248 | N | GLY | C | 10 | 15.831 | 22.187 | 43.437 | 1.00 | 25.07 | N |
| ATOM | 249 | CA | GLY | C | 10 | 15.318 | 22.677 | 44.713 | 1.00 | 26.18 | C |
| ATOM | 250 | C | GLY | C | 10 | 14.270 | 21.827 | 45.407 | 1.00 | 32.19 | C |
| ATOM | 251 | O | GLY | C | 10 | 13.872 | 22.155 | 46.523 | 1.00 | 33.38 | O |
| ATOM | 252 | O | LEU | C | 11 | 12.733 | 17.682 | 46.445 | 1.00 | 23.42 | O |
| ATOM | 253 | N | LEU | C | 11 | 13.800 | 20.734 | 44.774 | 1.00 | 27.61 | N |
| ATOM | 254 | CA | LEU | C | 11 | 12.791 | 19.852 | 45.384 | 1.00 | 25.12 | C |
| ATOM | 255 | C | LEU | C | 11 | 13.366 | 18.468 | 45.702 | 1.00 | 19.14 | C |
| ATOM | 256 | CB | LEU | C | 11 | 11.557 | 19.742 | 44.454 | 1.00 | 24.54 | C |
| ATOM | 257 | CG | LEU | C | 11 | 10.844 | 21.060 | 44.066 | 1.00 | 27.88 | C |
| ATOM | 258 | CD1 | LEU | C | 11 | 9.578 | 20.762 | 43.280 | 1.00 | 27.19 | C |
| ATOM | 259 | CD2 | LEU | C | 11 | 10.437 | 21.880 | 45.319 | 1.00 | 29.05 | C |
| ATOM | 260 | OXT | LEU | C | 11 | 14.454 | 18.150 | 45.177 | 1.00 | 24.96 | O |
| TER | 261 | | LEU | C | 11 | | | | | | |
| ATOM | 262 | N | LYS | D | 1 | 15.783 | 16.706 | 8.530 | 1.00 | 20.29 | N |
| ATOM | 263 | CA | LYS | D | 1 | 15.938 | 16.384 | 9.941 | 1.00 | 19.07 | C |
| ATOM | 264 | CB | LYS | D | 1 | 17.166 | 15.473 | 10.182 | 1.00 | 19.41 | C |
| ATOM | 265 | CG | LYS | D | 1 | 17.425 | 15.088 | 11.648 | 1.00 | 31.46 | C |
| ATOM | 266 | CD | LYS | D | 1 | 16.530 | 13.958 | 12.166 | 1.00 | 48.76 | C |
| ATOM | 267 | CE | LYS | D | 1 | 17.328 | 12.839 | 12.795 | 1.00 | 66.15 | C |
| ATOM | 268 | NZ | LYS | D | 1 | 16.467 | 11.664 | 13.104 | 1.00 | 74.58 | N |
| ATOM | 269 | C | LYS | D | 1 | 16.071 | 17.672 | 10.745 | 1.00 | 21.72 | C |
| ATOM | 270 | O | LYS | D | 1 | 16.972 | 18.470 | 10.498 | 1.00 | 20.46 | O |
| ATOM | 271 | N | VAL | D | 2 | 15.183 | 17.853 | 11.724 | 1.00 | 17.41 | N |
| ATOM | 272 | CA | VAL | D | 2 | 15.217 | 19.039 | 12.586 | 1.00 | 15.64 | C |
| ATOM | 273 | CB | VAL | D | 2 | 13.953 | 19.922 | 12.420 | 1.00 | 18.15 | C |
| ATOM | 274 | CG1 | VAL | D | 2 | 13.978 | 21.091 | 13.393 | 1.00 | 16.09 | C |
| ATOM | 275 | CG2 | VAL | D | 2 | 13.819 | 20.418 | 10.975 | 1.00 | 17.76 | C |
| ATOM | 276 | C | VAL | D | 2 | 15.425 | 18.567 | 14.023 | 1.00 | 17.74 | C |
| ATOM | 277 | O | VAL | D | 2 | 14.536 | 17.969 | 14.605 | 1.00 | 18.99 | O |
| ATOM | 278 | O | LYS | D | 3 | 16.361 | 20.568 | 16.649 | 1.00 | 17.72 | O |
| ATOM | 279 | N | LYS | D | 3 | 16.594 | 18.832 | 14.567 | 1.00 | 12.76 | N |
| ATOM | 280 | CA | LYS | D | 3 | 17.020 | 18.399 | 15.900 | 1.00 | 13.08 | C |
| ATOM | 281 | C | LYS | D | 3 | 16.551 | 19.384 | 16.940 | 1.00 | 17.80 | C |
| ATOM | 282 | CB | LYS | D | 3 | 18.568 | 18.326 | 15.954 | 1.00 | 16.86 | C |
| ATOM | 283 | CG | LYS | D | 3 | 19.160 | 17.186 | 15.135 | 1.00 | 24.47 | C |
| ATOM | 284 | CD | LYS | D | 3 | 20.660 | 17.213 | 15.172 | 1.00 | 37.64 | C |
| ATOM | 285 | CE | LYS | D | 3 | 21.236 | 16.321 | 14.101 | 1.00 | 50.49 | C |
| ATOM | 286 | NZ | LYS | D | 3 | 22.687 | 16.569 | 13.899 | 1.00 | 56.94 | N |
| ATOM | 287 | N | VAL | D | 4 | 16.373 | 18.889 | 18.166 | 1.00 | 14.47 | N |
| ATOM | 288 | CA | VAL | D | 4 | 15.924 | 19.654 | 19.331 | 1.00 | 13.40 | C |
| ATOM | 289 | CB | VAL | D | 4 | 14.445 | 19.330 | 19.665 | 1.00 | 15.88 | C |
| ATOM | 290 | CG1 | VAL | D | 4 | 13.974 | 20.081 | 20.912 | 1.00 | 14.47 | C |
| ATOM | 291 | CG2 | VAL | D | 4 | 13.526 | 19.614 | 18.469 | 1.00 | 15.31 | C |
| ATOM | 292 | C | VAL | D | 4 | 16.863 | 19.291 | 20.477 | 1.00 | 20.15 | C |
| ATOM | 293 | O | VAL | D | 4 | 17.151 | 18.102 | 20.681 | 1.00 | 19.09 | O |
| ATOM | 294 | N | TRP | D | 5 | 17.355 | 20.307 | 21.216 | 1.00 | 19.06 | N |
| ATOM | 295 | CA | ATRP | D | 5 | 18.279 | 20.089 | 22.331 | 0.50 | 20.49 | C |
| ATOM | 296 | CB | ATRP | D | 5 | 19.731 | 19.878 | 21.820 | 0.50 | 21.18 | C |
| ATOM | 297 | CG | ATRP | D | 5 | 20.749 | 19.730 | 22.921 | 0.50 | 24.24 | C |
| ATOM | 298 | CD1 | ATRP | D | 5 | 21.511 | 20.718 | 23.474 | 0.50 | 27.60 | C |
| ATOM | 299 | NE1 | ATRP | D | 5 | 22.264 | 20.213 | 24.513 | 0.50 | 27.71 | N |
| ATOM | 300 | CE2 | ATRP | D | 5 | 22.013 | 18.871 | 24.634 | 0.50 | 29.29 | C |
| ATOM | 301 | CD2 | ATRP | D | 5 | 21.044 | 18.536 | 23.660 | 0.50 | 24.80 | C |
| ATOM | 302 | CE3 | ATRP | D | 5 | 20.609 | 17.202 | 23.571 | 0.50 | 26.29 | C |
| ATOM | 303 | CZ3 | ATRP | D | 5 | 21.124 | 16.271 | 24.461 | 0.50 | 27.66 | C |
| ATOM | 304 | CH2 | ATRP | D | 5 | 22.103 | 16.626 | 25.397 | 0.50 | 28.73 | C |
| ATOM | 305 | CZ2 | ATRP | D | 5 | 22.571 | 17.918 | 25.495 | 0.50 | 28.83 | C |
| ATOM | 306 | C | TRP | D | 5 | 18.246 | 21.268 | 23.294 | 1.00 | 21.00 | C |
| ATOM | 307 | O | TRP | D | 5 | 18.232 | 22.429 | 22.865 | 1.00 | 18.15 | O |
| ATOM | 308 | CA | BTRP | D | 5 | 18.296 | 20.092 | 22.314 | 0.50 | 20.41 | C |
| ATOM | 309 | CB | BTRP | D | 5 | 19.735 | 19.939 | 21.742 | 0.50 | 20.99 | C |
| ATOM | 310 | CG | BTRP | D | 5 | 20.799 | 19.674 | 22.767 | 0.50 | 24.03 | C |
| ATOM | 311 | CD1 | BTRP | D | 5 | 21.158 | 18.462 | 23.280 | 0.50 | 27.49 | C |
| ATOM | 312 | NE1 | BTRP | D | 5 | 22.170 | 18.616 | 24.202 | 0.50 | 27.59 | N |
| ATOM | 313 | CE2 | BTRP | D | 5 | 22.476 | 19.949 | 24.310 | 0.50 | 29.12 | C |
| ATOM | 314 | CD2 | BTRP | D | 5 | 21.649 | 20.645 | 23.398 | 0.50 | 24.43 | C |
| ATOM | 315 | CE3 | BTRP | D | 5 | 21.774 | 22.044 | 23.303 | 0.50 | 25.82 | C |
| ATOM | 316 | CZ3 | BTRP | D | 5 | 22.716 | 22.688 | 24.096 | 0.50 | 27.50 | C |
| ATOM | 317 | CH2 | BTRP | D | 5 | 23.533 | 21.969 | 24.981 | 0.50 | 28.32 | C |
| ATOM | 318 | CZ2 | BTRP | D | 5 | 23.433 | 20.599 | 25.102 | 0.50 | 28.50 | C |
| ATOM | 319 | N | GLY | D | 6 | 18.287 | 20.961 | 24.579 | 1.00 | 14.03 | N |
| ATOM | 320 | CA | GLY | D | 6 | 18.354 | 21.999 | 25.596 | 1.00 | 14.10 | C |
| ATOM | 321 | C | GLY | D | 6 | 17.851 | 21.546 | 26.936 | 1.00 | 18.19 | C |
| ATOM | 322 | O | GLY | D | 6 | 17.913 | 20.355 | 27.278 | 1.00 | 16.01 | O |
| ATOM | 323 | N | SER | D | 7 | 17.398 | 22.512 | 27.717 | 1.00 | 15.88 | N |
| ATOM | 324 | CA | SER | D | 7 | 16.812 | 22.216 | 29.023 | 1.00 | 16.75 | C |
| ATOM | 325 | CB | SER | D | 7 | 17.876 | 22.144 | 30.123 | 1.00 | 19.30 | C |
| ATOM | 326 | OG | SER | D | 7 | 18.550 | 23.376 | 30.292 | 1.00 | 26.10 | O |
| ATOM | 327 | C | SER | D | 7 | 15.715 | 23.230 | 29.320 | 1.00 | 20.10 | C |
| ATOM | 328 | O | SER | D | 7 | 15.741 | 24.335 | 28.770 | 1.00 | 19.12 | 0 |
| ATOM | 329 | N | ILE | D | 8 | 14.735 | 22.845 | 30.146 | 1.00 | 16.19 | N |
| ATOM | 330 | CA | ILE | D | 8 | 13.602 | 23.705 | 30.433 | 1.00 | 18.42 | C |
| ATOM | 331 | CB | ILE | D | 8 | 12.335 | 22.889 | 30.844 | 1.00 | 21.09 | C |
| ATOM | 332 | CG1 | ILE | D | 8 | 11.975 | 21.846 | 29.766 | 1.00 | 21.78 | C |
| ATOM | 333 | CD1 | ILE | D | 8 | 10.839 | 20.887 | 30.136 | 1.00 | 32.23 | C |
| ATOM | 334 | CG2 | ILE | D | 8 | 11.144 | 23.835 | 31.153 | 1.00 | 20.50 | C |
| ATOM | 335 | C | ILE | D | 8 | 13.994 | 24.764 | 31.459 | 1.00 | 23.10 | C |
| ATOM | 336 | O | ILE | D | 8 | 14.393 | 24.407 | 32.563 | 1.00 | 20.46 | O |
| ATOM | 337 | N | LYS | D | 9 | 13.826 | 26.057 | 31.110 | 1.00 | 22.86 | N |
| ATOM | 338 | CA | LYS | D | 9 | 14.142 | 27.176 | 32.018 | 1.00 | 23.54 | C |
| ATOM | 339 | CB | LYS | D | 9 | 13.906 | 28.550 | 31.340 | 1.00 | 27.20 | C |
| ATOM | 340 | CG | LYS | D | 9 | 12.440 | 28.901 | 31.055 | 1.00 | 36.69 | C |
| ATOM | 341 | CD | LYS | D | 9 | 12.244 | 30.390 | 30.737 | 1.00 | 47.93 | C |
| ATOM | 342 | CE | LYS | D | 9 | 10.787 | 30.695 | 30.440 | 1.00 | 56.13 | C |
| ATOM | 343 | NZ | LYS | D | 9 | 10.598 | 32.039 | 29.821 | 1.00 | 51.12 | N |
| ATOM | 344 | C | LYS | D | 9 | 13.356 | 27.077 | 33.333 | 1.00 | 26.81 | C |
| ATOM | 345 | O | LYS | D | 9 | 12.186 | 26.702 | 33.323 | 1.00 | 26.88 | O |
| ATOM | 346 | N | GLY | D | 10 | 14.015 | 27.376 | 34.442 | 1.00 | 25.40 | N |
| ATOM | 347 | CA | GLY | D | 10 | 13.402 | 27.364 | 35.768 | 1.00 | 25.76 | C |
| ATOM | 348 | C | GLY | D | 10 | 13.306 | 26.016 | 36.462 | 1.00 | 30.84 | C |
| ATOM | 349 | O | GLY | D | 10 | 12.909 | 25.968 | 37.628 | 1.00 | 30.58 | O |
| ATOM | 350 | O | LEU | D | 11 | 15.944 | 23.444 | 36.058 | 1.00 | 21.41 | O |
| ATOM | 351 | N | LEU | D | 11 | 13.672 | 24.909 | 35.781 | 1.00 | 26.65 | N |
| ATOM | 352 | CA | LEU | D | 11 | 13.588 | 23.563 | 36.378 | 1.00 | 24.24 | C |
| ATOM | 353 | C | LEU | D | 11 | 14.960 | 22.932 | 36.615 | 1.00 | 24.70 | C |
| ATOM | 354 | CB | LEU | D | 11 | 12.721 | 22.636 | 35.491 | 1.00 | 23.45 | C |
| ATOM | 355 | CG | LEU | D | 11 | 11.288 | 23.095 | 35.162 | 1.00 | 25.24 | C |
| ATOM | 356 | CD1 | LEU | D | 11 | 10.551 | 22.012 | 34.395 | 1.00 | 24.35 | C |
| ATOM | 357 | CD2 | LEU | D | 11 | 10.492 | 23.376 | 36.426 | 1.00 | 26.73 | C |
| ATOM | 358 | OXT | LEU | D | 11 | 15.053 | 21.897 | 37.320 | 1.00 | 28.00 | O |
| TER | 359 | | LEU | D | 11 | | | | | | |
| ATOM | 360 | N | LYS | E | 1 | 6.079 | 13.424 | 54.931 | 1.00 | 18.83 | N |
| ATOM | 361 | CA | LYS | E | 1 | 6.349 | 13.243 | 53.504 | 1.00 | 18.59 | C |
| ATOM | 362 | CB | LYS | E | 1 | 6.227 | 11.744 | 53.136 | 1.00 | 21.18 | C |
| ATOM | 363 | CG | LYS | E | 1 | 6.456 | 11.428 | 51.665 | 1.00 | 32.51 | C |
| ATOM | 364 | CD | LYS | E | 1 | 7.503 | 10.340 | 51.564 | 1.00 | 44.30 | C |
| ATOM | 365 | CE | LYS | E | 1 | 7.925 | 10.042 | 50.162 | 1.00 | 56.00 | C |
| ATOM | 366 | NZ | LYS | E | 1 | 9.313 | 9.514 | 50.107 | 1.00 | 68.84 | N |
| ATOM | 367 | C | LYS | E | 1 | 5.329 | 14.076 | 52.697 | 1.00 | 19.31 | C |
| ATOM | 368 | O | LYS | E | 1 | 4.127 | 14.012 | 52.968 | 1.00 | 18.26 | O |
| ATOM | 369 | N | VAL | E | 2 | 5.813 | 14.826 | 51.707 | 1.00 | 12.76 | N |
| ATOM | 370 | CA | VAL | E | 2 | 4.978 | 15.667 | 50.863 | 1.00 | 12.69 | C |
| ATOM | 371 | CB | VAL | E | 2 | 5.282 | 17.166 | 51.089 | 1.00 | 15.65 | C |
| ATOM | 372 | CG1 | VAL | E | 2 | 4.480 | 18.022 | 50.111 | 1.00 | 15.27 | C |
| ATOM | 373 | CG2 | VAL | E | 2 | 4.971 | 17.572 | 52.529 | 1.00 | 14.44 | C |
| ATOM | 374 | C | VAL | E | 2 | 5.168 | 15.250 | 49.418 | 1.00 | 15.34 | C |
| ATOM | 375 | O | VAL | E | 2 | 6.243 | 15.459 | 48.859 | 1.00 | 14.04 | O |
| ATOM | 376 | N | LYS | E | 3 | 4.149 | 14.598 | 48.839 | 1.00 | 12.02 | N |
| ATOM | 377 | CA | LYS | E | 3 | 4.222 | 14.096 | 47.475 | 1.00 | 12.32 | C |
| ATOM | 378 | CB | LYS | E | 3 | 3.259 | 12.912 | 47.274 | 1.00 | 17.06 | C |
| ATOM | 379 | CG | LYS | E | 3 | 3.828 | 11.625 | 47.879 | 1.00 | 39.48 | C |
| ATOM | 380 | CD | LYS | E | 3 | 4.609 | 10.808 | 46.843 | 1.00 | 33.22 | C |
| ATOM | 381 | CE | LYS | E | 3 | 4.468 | 9.342 | 47.128 | 1.00 | 33.92 | C |
| ATOM | 382 | NZ | LYS | E | 3 | 5.202 | 8.909 | 48.358 | 1.00 | 51.42 | N |
| ATOM | 383 | C | LYS | E | 3 | 3.937 | 15.177 | 46.448 | 1.00 | 14.62 | C |
| ATOM | 384 | O | LYS | E | 3 | 3.197 | 16.110 | 46.728 | 1.00 | 11.80 | O |
| ATOM | 385 | N | VAL | E | 4 | 4.527 | 15.023 | 45.258 | 1.00 | 12.42 | N |
| ATOM | 386 | CA | VAL | E | 4 | 4.406 | 15.943 | 44.134 | 1.00 | 13.02 | C |
| ATOM | 387 | CB | VAL | E | 4 | 5.704 | 16.777 | 43.947 | 1.00 | 15.62 | C |
| ATOM | 388 | CG1 | VAL | E | 4 | 5.598 | 17.693 | 42.730 | 1.00 | 14.41 | C |
| ATOM | 389 | CG2 | VAL | E | 4 | 6.033 | 17.580 | 45.200 | 1.00 | 14.79 | C |
| ATOM | 390 | C | VAL | E | 4 | 4.119 | 15.080 | 42.924 | 1.00 | 17.87 | C |
| ATOM | 391 | O | VAL | E | 4 | 4.840 | 14.103 | 42.687 | 1.00 | 18.13 | O |
| ATOM | 392 | N | TRP | E | 5 | 3.049 | 15.403 | 42.180 | 1.00 | 13.35 | N |
| ATOM | 393 | CA | TRP | E | 5 | 2.691 | 14.597 | 41.022 | 1.00 | 12.13 | C |
| ATOM | 394 | CB | TRP | E | 5 | 1.973 | 13.277 | 41.461 | 1.00 | 10.30 | C |
| ATOM | 395 | CG | TRP | E | 5 | 0.546 | 13.453 | 41.895 | 1.00 | 10.95 | C |
| ATOM | 396 | CD1 | TRP | E | 5 | −0.577 | 13.187 | 41.157 | 1.00 | 14.06 | C |
| ATOM | 397 | NE1 | TRP | E | 5 | −1.708 | 13.491 | 41.891 | 1.00 | 12.85 | N |
| ATOM | 398 | CE2 | TRP | E | 5 | −1.326 | 14.005 | 43.108 | 1.00 | 13.92 | C |
| ATOM | 399 | CD2 | TRP | E | 5 | 0.088 | 13.969 | 43.157 | 1.00 | 9.92 | C |
| ATOM | 400 | CE3 | TRP | E | 5 | 0.742 | 14.416 | 44.326 | 1.00 | 10.63 | C |
| ATOM | 401 | CZ3 | TRP | E | 5 | −0.028 | 14.882 | 45.385 | 1.00 | 11.67 | C |
| ATOM | 402 | CH2 | TRP | E | 5 | −1.436 | 14.843 | 45.328 | 1.00 | 12.29 | C |
| ATOM | 403 | CZ2 | TRP | E | 5 | −2.102 | 14.443 | 44.190 | 1.00 | 12.85 | C |
| ATOM | 404 | C | TRP | E | 5 | 1.807 | 15.374 | 40.056 | 1.00 | 15.59 | C |
| ATOM | 405 | O | TRP | E | 5 | 1.070 | 16.269 | 40.455 | 1.00 | 14.03 | O |
| ATOM | 406 | N | GLY | E | 6 | 1.838 | 14.955 | 38.798 | 1.00 | 11.46 | N |
| ATOM | 407 | CA | GLY | E | 6 | 0.981 | 15.526 | 37.774 | 1.00 | 11.35 | C |
| ATOM | 408 | C | GLY | E | 6 | 1.684 | 15.649 | 36.448 | 1.00 | 14.43 | C |
| ATOM | 409 | O | GLY | E | 6 | 2.488 | 14.793 | 36.095 | 1.00 | 11.28 | O |
| ATOM | 410 | N | SER | E | 7 | 1.370 | 16.717 | 35.708 | 1.00 | 14.32 | N |
| ATOM | 411 | CA | SER | E | 7 | 1.989 | 16.949 | 34.418 | 1.00 | 14.36 | C |
| ATOM | 412 | CB | SER | E | 7 | 1.175 | 16.308 | 33.291 | 1.00 | 18.90 | C |
| ATOM | 413 | OG | SER | E | 7 | −0.140 | 16.846 | 33.245 | 1.00 | 19.06 | O |
| ATOM | 414 | C | SER | E | 7 | 2.198 | 18.435 | 34.192 | 1.00 | 17.22 | C |
| ATOM | 415 | O | SER | E | 7 | 1.509 | 19.264 | 34.801 | 1.00 | 14.93 | O |
| ATOM | 416 | N | ILE | E | 8 | 3.229 | 18.776 | 33.399 | 1.00 | 15.15 | N |
| ATOM | 417 | CA | ILE | E | 8 | 3.550 | 20.174 | 33.114 | 1.00 | 15.71 | C |
| ATOM | 418 | CB | ILE | E | 8 | 5.038 | 20.351 | 32.727 | 1.00 | 17.97 | C |
| ATOM | 419 | CG1 | ILE | E | 8 | 5.952 | 19.843 | 33.904 | 1.00 | 18.60 | C |
| ATOM | 420 | CD1 | ILE | E | 8 | 7.434 | 19.866 | 33.659 | 1.00 | 27.60 | C |
| ATOM | 421 | CG2 | ILE | E | 8 | 5.331 | 21.853 | 32.344 | 1.00 | 17.61 | C |
| ATOM | 422 | C | ILE | E | 8 | 2.587 | 20.711 | 32.044 | 1.00 | 22.15 | C |
| ATOM | 423 | O | ILE | E | 8 | 2.541 | 20.164 | 30.941 | 1.00 | 20.99 | O |
| ATOM | 424 | N | LYS | E | 9 | 1.824 | 21.766 | 32.380 | 1.00 | 21.70 | N |
| ATOM | 425 | CA | LYS | E | 9 | 0.866 | 22.388 | 31.453 | 1.00 | 22.40 | C |
| ATOM | 426 | CB | LYS | E | 9 | 0.104 | 23.556 | 32.127 | 1.00 | 25.00 | C |
| ATOM | 427 | CG | LYS | E | 9 | 0.952 | 24.791 | 32.429 | 1.00 | 37.43 | C |
| ATOM | 428 | CD | LYS | E | 9 | 0.100 | 26.024 | 32.719 | 1.00 | 42.27 | C |
| ATOM | 429 | CE | LYS | E | 9 | 0.940 | 27.192 | 33.176 | 1.00 | 49.38 | C |
| ATOM | 430 | NZ | LYS | E | 9 | 1.782 | 27.741 | 32.086 | 1.00 | 57.22 | N |
| ATOM | 431 | C | LYS | E | 9 | 1.544 | 22.839 | 30.147 | 1.00 | 26.22 | C |
| ATOM | 432 | O | LYS | E | 9 | 2.669 | 23.330 | 30.168 | 1.00 | 26.71 | O |
| ATOM | 433 | N | GLY | E | 10 | 0.873 | 22.610 | 29.038 | 1.00 | 24.65 | N |
| ATOM | 434 | CA | GLY | E | 10 | 1.344 | 23.022 | 27.721 | 1.00 | 25.96 | C |
| ATOM | 435 | C | GLY | E | 10 | 2.283 | 22.055 | 27.022 | 1.00 | 31.77 | C |
| ATOM | 436 | O | GLY | E | 10 | 2.613 | 22.281 | 25.857 | 1.00 | 31.72 | O |
| ATOM | 437 | O | LEU | E | 11 | 1.894 | 18.401 | 27.365 | 1.00 | 25.78 | O |
| ATOM | 438 | N | LEU | E | 11 | 2.720 | 20.967 | 27.707 | 1.00 | 26.26 | N |
| ATOM | 439 | CA | LEU | E | 11 | 3.654 | 19.997 | 27.116 | 1.00 | 23.78 | C |
| ATOM | 440 | C | LEU | E | 11 | 3.004 | 18.641 | 26.845 | 1.00 | 23.23 | C |
| ATOM | 441 | CB | LEU | E | 11 | 4.897 | 19.836 | 28.023 | 1.00 | 23.52 | C |
| ATOM | 442 | CG | LEU | E | 11 | 5.704 | 21.115 | 28.359 | 1.00 | 27.16 | C |
| ATOM | 443 | CD1 | LEU | E | 11 | 6.979 | 20.761 | 29.144 | 1.00 | 26.42 | C |
| ATOM | 444 | CD2 | LEU | E | 11 | 6.104 | 21.880 | 27.083 | 1.00 | 30.45 | C |
| ATOM | 445 | OXT | LEU | E | 11 | 3.597 | 17.820 | 26.109 | 1.00 | 25.63 | O |
| TER | 446 | | LEU | E | 11 | | | | | | |
| ATOM | 447 | O | LYS | F | 1 | −0.218 | 18.764 | 37.344 | 1.00 | 14.89 | O |
| ATOM | 448 | N | LYS | F | 1 | −0.705 | 20.751 | 35.307 | 1.00 | 19.07 | N |
| ATOM | 449 | CA | LYS | F | 1 | −0.939 | 20.987 | 36.724 | 1.00 | 18.11 | C |
| ATOM | 450 | C | LYS | F | 1 | −0.127 | 19.976 | 37.561 | 1.00 | 17.65 | C |
| ATOM | 451 | CB | LYS | F | 1 | −2.434 | 20.864 | 37.041 | 1.00 | 20.39 | C |
| ATOM | 452 | CG | LYS | F | 1 | −2.757 | 21.157 | 38.504 | 1.00 | 29.12 | C |
| ATOM | 453 | CD | LYS | F | 1 | −4.224 | 21.023 | 38.766 | 1.00 | 41.56 | C |
| ATOM | 454 | CE | LYS | F | 1 | −4.879 | 22.346 | 39.094 | 1.00 | 58.69 | C |
| ATOM | 455 | NZ | LYS | F | 1 | −6.341 | 22.185 | 39.348 | 1.00 | 68.54 | N |
| ATOM | 456 | N | VAL | F | 2 | 0.656 | 20.495 | 38.510 | 1.00 | 13.36 | N |
| ATOM | 457 | CA | VAL | F | 2 | 1.488 | 19.676 | 39.386 | 1.00 | 12.54 | C |
| ATOM | 458 | CB | VAL | F | 2 | 2.992 | 19.927 | 39.195 | 1.00 | 13.96 | C |
| ATOM | 459 | CG1 | VAL | F | 2 | 3.800 | 19.107 | 40.204 | 1.00 | 13.77 | C |
| ATOM | 460 | CG2 | VAL | F | 2 | 3.418 | 19.603 | 37.758 | 1.00 | 12.05 | C |
| ATOM | 461 | C | VAL | F | 2 | 1.011 | 19.832 | 40.820 | 1.00 | 16.08 | C |
| ATOM | 462 | O | VAL | F | 2 | 1.202 | 20.889 | 41.425 | 1.00 | 16.05 | O |
| ATOM | 463 | N | LYS | F | 3 | 0.345 | 18.790 | 41.335 | 1.00 | 10.37 | N |
| ATOM | 464 | CA | LYS | F | 3 | −0.262 | 18.807 | 42.668 | 1.00 | 10.43 | C |
| ATOM | 465 | C | LYS | F | 3 | 0.779 | 18.472 | 43.719 | 1.00 | 15.04 | C |
| ATOM | 466 | O | LYS | F | 3 | 1.757 | 17.800 | 43.426 | 1.00 | 14.39 | O |
| ATOM | 467 | CB | LYS | F | 3 | −1.406 | 17.793 | 42.717 | 1.00 | 13.50 | C |
| ATOM | 468 | CG | LYS | F | 3 | −2.615 | 18.219 | 41.909 | 1.00 | 20.64 | C |
| ATOM | 469 | CD | LYS | F | 3 | −3.699 | 17.189 | 41.989 | 1.00 | 31.97 | C |
| ATOM | 470 | CE | LYS | F | 3 | −4.937 | 17.675 | 41.264 | 1.00 | 43.59 | C |
| ATOM | 471 | NZ | LYS | F | 3 | −6.188 | 17.262 | 41.962 | 1.00 | 52.20 | N |
| ATOM | 472 | N | VAL | F | 4 | 0.557 | 18.952 | 44.937 | 1.00 | 12.00 | N |
| ATOM | 473 | CA | VAL | F | 4 | 1.411 | 18.749 | 46.104 | 1.00 | 11.16 | C |
| ATOM | 474 | CB | VAL | F | 4 | 2.206 | 20.042 | 46.424 | 1.00 | 14.14 | C |
| ATOM | 475 | CG1 | VAL | F | 4 | 3.024 | 19.883 | 47.693 | 1.00 | 14.17 | C |
| ATOM | 476 | CG2 | VAL | F | 4 | 3.107 | 20.443 | 45.247 | 1.00 | 13.07 | C |
| ATOM | 477 | C | VAL | F | 4 | 0.484 | 18.352 | 47.251 | 1.00 | 16.88 | C |
| ATOM | 478 | O | VAL | F | 4 | −0.576 | 18.959 | 47.408 | 1.00 | 16.16 | O |
| ATOM | 479 | N | TRP | F | 5 | 0.869 | 17.334 | 48.045 | 1.00 | 14.77 | N |
| ATOM | 480 | CA | TRP | F | 5 | 0.032 | 16.902 | 49.159 | 1.00 | 16.33 | C |
| ATOM | 481 | CB | TRP | F | 5 | −1.214 | 16.117 | 48.647 | 1.00 | 17.08 | C |
| ATOM | 482 | CG | TRP | F | 5 | −2.082 | 15.567 | 49.742 | 1.00 | 20.07 | C |
| ATOM | 483 | CD1 | TRP | F | 5 | −2.927 | 16.270 | 50.556 | 1.00 | 23.48 | C |
| ATOM | 484 | NE1 | TRP | F | 5 | −3.530 | 15.420 | 51.454 | 1.00 | 23.62 | N |
| ATOM | 485 | CE2 | TRP | F | 5 | −3.099 | 14.138 | 51.215 | 1.00 | 24.90 | C |
| ATOM | 486 | CD2 | TRP | F | 5 | −2.196 | 14.193 | 50.131 | 1.00 | 20.37 | C |
| ATOM | 487 | CE3 | TRP | F | 5 | −1.653 | 12.990 | 49.637 | 1.00 | 21.94 | C |
| ATOM | 488 | CZ3 | TRP | F | 5 | −1.957 | 11.804 | 50.288 | 1.00 | 23.66 | C |
| ATOM | 489 | CH2 | TRP | F | 5 | −2.854 | 11.778 | 51.366 | 1.00 | 24.24 | C |
| ATOM | 490 | CZ2 | TRP | F | 5 | −3.450 | 12.932 | 51.837 | 1.00 | 24.27 | C |
| ATOM | 491 | C | TRP | F | 5 | 0.786 | 16.047 | 50.131 | 1.00 | 17.68 | C |
| ATOM | 492 | O | TRP | F | 5 | 1.478 | 15.125 | 49.732 | 1.00 | 15.11 | O |
| ATOM | 493 | N | GLY | F | 6 | 0.590 | 16.317 | 51.409 | 1.00 | 15.00 | N |
| ATOM | 494 | CA | GLY | F | 6 | 1.114 | 15.467 | 52.454 | 1.00 | 14.41 | C |
| ATOM | 495 | C | GLY | F | 6 | 1.127 | 16.166 | 53.786 | 1.00 | 18.67 | C |
| ATOM | 496 | O | GLY | F | 6 | 0.2591 | 17.006 | 54.091 | 1.00 | 16.57 | O |
| ATOM | 497 | N | SER | F | 7 | 2.143 | 15.823 | 54.576 | 1.00 | 14.40 | N |
| ATOM | 498 | CA | SER | F | 7 | 2.337 | 16.421 | 55.881 | 1.00 | 15.02 | C |
| ATOM | 499 | CB | SER | F | 7 | 1.552 | 15.665 | 56.957 | 1.00 | 18.42 | C |
| ATOM | 500 | OG | SER | F | 7 | 1.969 | 14.315 | 57.053 | 1.00 | 23.61 | O |
| ATOM | 501 | C | SER | F | 7 | 3.827 | 16.501 | 56.181 | 1.00 | 19.43 | C |
| ATOM | 502 | O | SER | F | 7 | 4.616 | 15.746 | 55.608 | 1.00 | 18.81 | O |
| ATOM | 503 | N | ILE | F | 8 | 4.226 | 17.468 | 57.009 | 1.00 | 17.56 | N |
| ATOM | 504 | CA | ILE | F | 8 | 5.647 | 17.658 | 57.320 | 1.00 | 18.06 | C |
| ATOM | 505 | CB | ILE | F | 8 | 5.929 | 19.114 | 57.771 | 1.00 | 20.62 | C |
| ATOM | 506 | CG1 | ILE | F | 8 | 5.493 | 20.122 | 56.684 | 1.00 | 20.03 | C |
| ATOM | 507 | CD1 | ILE | F | 8 | 5.552 | 21.635 | 57.113 | 1.00 | 28.20 | C |
| ATOM | 508 | CG2 | ILE | F | 8 | 7.421 | 19.300 | 58.159 | 1.00 | 21.31 | C |
| ATOM | 509 | C | ILE | F | 8 | 6.105 | 16.612 | 58.346 | 1.00 | 22.34 | C |
| ATOM | 510 | O | ILE | F | 8 | 5.527 | 16.528 | 59.427 | 1.00 | 20.30 | O |
| ATOM | 511 | N | LYS | F | 9 | 7.137 | 15.817 | 58.005 | 1.00 | 21.63 | N |
| ATOM | 512 | CA | LYS | F | 9 | 7.662 | 14.787 | 58.922 | 1.00 | 23.40 | C |
| ATOM | 513 | CB | LYS | F | 9 | 8.838 | 13.988 | 58.300 | 1.00 | 27.83 | C |
| ATOM | 514 | CG | LYS | F | 9 | 10.107 | 14.798 | 58.028 | 1.00 | 43.99 | C |
| ATOM | 515 | CD | LYS | F | 9 | 11.305 | 13.919 | 57.709 | 1.00 | 55.23 | C |
| ATOM | 516 | CE | LYS | F | 9 | 12.526 | 14.759 | 57.403 | 1.00 | 66.81 | C |
| ATOM | 517 | NZ | LYS | F | 9 | 13.668 | 13.928 | 56.942 | 1.00 | 77.85 | N |
| ATOM | 518 | C | LYS | F | 9 | 8.083 | 15.384 | 60.272 | 1.00 | 25.16 | C |
| ATOM | 519 | O | LYS | F | 9 | 8.634 | 16.485 | 60.307 | 1.00 | 24.07 | O |
| ATOM | 520 | N | GLY | F | 10 | 7.780 | 14.670 | 61.349 | 1.00 | 23.07 | N |
| ATOM | 521 | CA | GLY | F | 10 | 8.153 | 15.071 | 62.706 | 1.00 | 23.98 | C |
| ATOM | 522 | C | GLY | F | 10 | 7.251 | 16.082 | 63.391 | 1.00 | 30.42 | C |
| ATOM | 523 | O | GLY | F | 10 | 7.483 | 16.391 | 64.559 | 1.00 | 30.99 | O |
| ATOM | 524 | O | LEU | F | 11 | 3.603 | 15.976 | 62.862 | 1.00 | 20.88 | O |
| ATOM | 525 | N | LEU | F | 11 | 6.227 | 16.628 | 62.684 | 1.00 | 26.08 | N |
| ATOM | 526 | CA | LEU | F | 11 | 5.304 | 17.616 | 63.273 | 1.00 | 23.66 | C |
| ATOM | 527 | C | LEU | F | 11 | 3.889 | 17.053 | 63.430 | 1.00 | 15.77 | C |
| ATOM | 528 | CB | LEU | F | 11 | 5.280 | 18.897 | 62.420 | 1.00 | 23.33 | C |
| ATOM | 529 | CG | LEU | F | 11 | 6.627 | 19.606 | 62.149 | 1.00 | 26.95 | C |
| ATOM | 530 | CD1 | LEU | F | 11 | 6.398 | 20.918 | 61.396 | 1.00 | 26.51 | C |
| ATOM | 531 | CD2 | LEU | F | 11 | 7.369 | 19.932 | 63.449 | 1.00 | 28.94 | C |
| ATOM | 532 | OXT | LEU | F | 11 | 3.059 | 17.672 | 64.122 | 1.00 | 26.14 | O |
| TER | 533 | | LEU | F | 11 | | | | | | |
| ATOM | 596 | O | LYS | Q | 1 | 11.422 | 13.670 | 44.264 | 1.00 | 13.54 | O |
| ATOM | 597 | N | LYS | Q | 1 | 13.264 | 14.724 | 46.092 | 1.00 | 19.66 | N |
| ATOM | 598 | CA | LYS | Q | 1 | 13.600 | 14.540 | 44.672 | 1.00 | 18.75 | C |
| ATOM | 599 | C | LYS | Q | 1 | 12.300 | 14.424 | 43.878 | 1.00 | 17.39 | C |
| ATOM | 600 | CB | LYS | Q | 1 | 14.447 | 13.271 | 44.470 | 1.00 | 22.14 | C |
| ATOM | 601 | O | VAL | Q | 2 | 12.076 | 15.740 | 39.923 | 1.00 | 19.04 | O |
| ATOM | 602 | N | VAL | Q | 2 | 12.168 | 15.204 | 42.801 | 1.00 | 13.38 | N |
| ATOM | 603 | CA | VAL | Q | 2 | 10.968 | 15.212 | 41.984 | 1.00 | 13.95 | C |
| ATOM | 604 | C | VAL | Q | 2 | 11.404 | 14.919 | 40.549 | 1.00 | 19.38 | C |
| ATOM | 605 | CB | VAL | Q | 2 | 10.156 | 16.534 | 42.120 | 1.00 | 17.31 | C |
| ATOM | 606 | CG1 | VAL | Q | 2 | 8.937 | 16.511 | 41.171 | 1.00 | 17.01 | C |
| ATOM | 607 | CG2 | VAL | Q | 2 | 9.679 | 16.741 | 43.571 | 1.00 | 16.21 | C |
| ATOM | 608 | N | LYS | Q | 3 | 11.040 | 13.750 | 40.050 | 1.00 | 15.63 | N |
| ATOM | 609 | CA | LYS | Q | 3 | 11.380 | 13.288 | 38.699 | 1.00 | 15.71 | C |
| ATOM | 610 | C | LYS | Q | 3 | 10.441 | 13.930 | 37.661 | 1.00 | 17.14 | C |
| ATOM | 611 | O | LYS | Q | 3 | 9.278 | 14.180 | 37.956 | 1.00 | 13.61 | O |
| ATOM | 612 | CB | LYS | Q | 3 | 11.198 | 11.740 | 38.649 | 1.00 | 18.62 | C |
| ATOM | 613 | CG | LYS | Q | 3 | 11.612 | 11.063 | 37.370 | 1.00 | 32.55 | C |
| ATOM | 614 | CD | LYS | Q | 3 | 11.203 | 9.605 | 37.405 | 1.00 | 35.88 | C |
| ATOM | 615 | CE | LYS | Q | 3 | 11.372 | 8.996 | 36.042 | 1.00 | 45.27 | C |
| ATOM | 616 | NZ | LYS | Q | 3 | 11.395 | 7.514 | 36.112 | 1.00 | 55.94 | N |
| ATOM | 617 | N | VAL | Q | 4 | 10.959 | 14.146 | 36.438 | 1.00 | 13.85 | N |
| ATOM | 618 | CA | VAL | Q | 4 | 10.236 | 14.688 | 35.302 | 1.00 | 12.61 | C |
| ATOM | 619 | C | VAL | Q | 4 | 10.543 | 13.784 | 34.121 | 1.00 | 17.86 | C |
| ATOM | 620 | O | VAL | Q | 4 | 11.716 | 13.481 | 33.886 | 1.00 | 18.44 | O |
| ATOM | 621 | CB | VAL | Q | 4 | 10.642 | 16.163 | 35.002 | 1.00 | 14.86 | C |
| ATOM | 622 | CG1 | VAL | Q | 4 | 9.831 | 16.715 | 33.82 | 1.00 | 13.70 | C |
| ATOM | 623 | CG2 | VAL | Q | 4 | 10.448 | 17.050 | 36.228 | 1.00 | 14.37 | C |
| ATOM | 624 | N | TRP | Q | 5 | 9.509 | 13.357 | 33.364 | 1.00 | 14.73 | N |
| ATOM | 625 | CA | TRP | Q | 5 | 9.744 | 12.477 | 32.212 | 1.00 | 14.32 | C |
| ATOM | 626 | C | TRP | Q | 5 | 8.597 | 12.509 | 31.225 | 1.00 | 17.08 | C |
| ATOM | 627 | O | TRP | Q | 5 | 7.449 | 12.716 | 31.611 | 1.00 | 15.76 | O |
| ATOM | 628 | CB | TRP | Q | 5 | 10.082 | 11.015 | 32.670 | 1.00 | 12.85 | C |
| ATOM | 629 | CG | TRP | Q | 5 | 8.898 | 10.170 | 33.028 | 1.00 | 13.52 | C |
| ATOM | 630 | CD1 | TRP | Q | 5 | 8.314 | 9.210 | 32.251 | 1.00 | 16.49 | C |
| ATOM | 631 | CD2 | TRP | Q | 5 | 8.113 | 10.250 | 34.232 | 1.00 | 12.90 | C |
| ATOM | 632 | NE1 | TRP | Q | 5 | 7.205 | 8.699 | 32.884 | 1.00 | 15.57 | N |
| ATOM | 633 | CE2 | TRP | Q | 5 | 7.039 | 9.341 | 34.091 | 1.00 | 16.86 | C |
| ATOM | 634 | CE3 | TRP | Q | 5 | 8.189 | 11.041 | 35.404 | 1.00 | 13.32 | C |
| ATOM | 635 | CZ2 | TRP | Q | 5 | 6.052 | 9.177 | 35.086 | 1.00 | 15.35 | C |
| ATOM | 636 | CZ3 | TRP | Q | 5 | 7.220 | 10.870 | 36.386 | 1.00 | 14.86 | C |
| ATOM | 637 | CH2 | TRP | Q | 5 | 6.175 | 9.932 | 36.230 | 1.00 | 15.43 | C |
| ATOM | 638 | N | GLY | Q | 6 | 8.909 | 12.256 | 29.958 | 1.00 | 14.61 | N |
| ATOM | 639 | CA | GLY | Q | 6 | 7.871 | 12.156 | 28.938 | 1.00 | 14.27 | C |
| ATOM | 640 | C | GLY | Q | 6 | 8.333 | 12.639 | 27.590 | 1.00 | 16.75 | C |
| ATOM | 641 | O | GLY | Q | 6 | 9.491 | 12.440 | 27.230 | 1.00 | 16.70 | O |
| ATOM | 642 | N | SER | Q | 7 | 7.436 | 13.309 | 26.862 | 1.00 | 12.99 | N |
| ATOM | 643 | CA | SER | Q | 7 | 7.725 | 13.842 | 25.542 | 1.00 | 13.28 | C |
| ATOM | 644 | C | SER | Q | 7 | 6.848 | 15.048 | 25.277 | 1.00 | 17.77 | C |
| ATOM | 645 | O | SER | Q | 7 | 5.794 | 15.180 | 25.885 | 1.00 | 17.17 | O |
| ATOM | 646 | CB | SER | Q | 7 | 7.551 | 12.766 | 24.463 | 1.00 | 16.86 | C |
| ATOM | 647 | OG | SER | Q | 7 | 6.241 | 12.228 | 24.458 | 1.00 | 26.43 | O |
| ATOM | 648 | N | ILE | Q | 8 | 7.320 | 15.981 | 24.440 | 1.00 | 16.02 | N |
| ATOM | 649 | CA | ILE | Q | 8 | 6.545 | 17.195 | 24.137 | 1.00 | 15.35 | C |
| ATOM | 650 | C | ILE | Q | 8 | 5.463 | 16.871 | 23.087 | 1.00 | 20.02 | C |
| ATOM | 651 | O | ILE | Q | 8 | 5.786 | 16.410 | 21.990 | 1.00 | 16.98 | O |
| ATOM | 652 | CB | ILE | Q | 8 | 7.481 | 18.354 | 23.699 | 1.00 | 17.35 | C |
| ATOM | 653 | CG1 | ILE | Q | 8 | 8.457 | 18.725 | 24.857 | 1.00 | 16.28 | C |
| ATOM | 654 | CG2 | ILE | Q | 8 | 6.655 | 19.587 | 23.265 | 1.00 | 17.59 | C |
| ATOM | 655 | CD1 | ILE | Q | 8 | 9.606 | 19.690 | 24.425 | 1.00 | 24.35 | C |
| ATOM | 656 | N | LYS | Q | 9 | 4.193 | 17.108 | 23.434 | 1.00 | 19.74 | N |
| ATOM | 657 | CA | LYS | Q | 9 | 3.061 | 16.844 | 22.531 | 1.00 | 19.94 | C |
| ATOM | 658 | C | LYS | Q | 9 | 3.200 | 17.573 | 21.191 | 1.00 | 23.22 | C |
| ATOM | 659 | O | LYS | Q | 9 | 3.694 | 18.699 | 21.153 | 1.00 | 20.63 | O |
| ATOM | 660 | CB | LYS | Q | 9 | 1.718 | 17.165 | 23.217 | 1.00 | 23.17 | C |
| ATOM | 661 | CG | LYS | Q | 9 | 1.414 | 18.644 | 23.441 | 1.00 | 41.25 | C |
| ATOM | 662 | CD | LYS | Q | 9 | −0.084 | 18.892 | 23.765 | 1.00 | 52.04 | C |
| ATOM | 663 | CE | LYS | Q | 9 | −1.079 | 18.410 | 22.709 | 1.00 | 62.44 | C |
| ATOM | 664 | NZ | LYS | Q | 9 | −2.484 | 18.748 | 23.048 | 1.00 | 72.45 | N |
| ATOM | 665 | N | GLY | Q | 10 | 2.825 | 16.893 | 20.115 | 1.00 | 22.11 | N |
| ATOM | 666 | CA | GLY | Q | 10 | 2.870 | 17.454 | 18.766 | 1.00 | 22.73 | C |
| ATOM | 667 | C | GLY | Q | 10 | 4.211 | 17.404 | 18.050 | 1.00 | 26.41 | C |
| ATOM | 668 | O | GLY | Q | 10 | 4.275 | 17.758 | 16.870 | 1.00 | 24.19 | O |
| ATOM | 669 | O | LEU | Q | 11 | 8.195 | 15.291 | 17.236 | 1.00 | 20.03 | O |
| ATOM | 670 | N | LEU | Q | 11 | 5.302 | 16.955 | 18.737 | 1.00 | 22.62 | N |
| ATOM | 671 | CA | LEU | Q | 11 | 6.642 | 16.906 | 18.115 | 1.00 | 23.00 | C |
| ATOM | 672 | C | LEU | Q | 11 | 7.168 | 15.480 | 17.935 | 1.00 | 23.79 | C |
| ATOM | 673 | CB | LEU | Q | 11 | 7.652 | 17.760 | 18.930 | 1.00 | 22.92 | C |
| ATOM | 674 | CG | LEU | Q | 11 | 7.243 | 19.197 | 19.300 | 1.00 | 26.24 | C |
| ATOM | 675 | CD2 | LEU | Q | 11 | 6.847 | 20.013 | 18.042 | 1.00 | 28.14 | C |
| ATOM | 676 | CD1 | LEU | Q | 11 | 8.377 | 19.909 | 20.008 | 1.00 | 25.85 | C |
| ATOM | 677 | OXT | LEU | Q | 11 | 6.558 | 14.547 | 18.497 | 1.00 | 20.72 | O |
| TER | 678 | | LEU | Q | 11 | | | | | | |
| ATOM | 679 | O | LYS | R | 1 | 4.824 | 13.643 | 28.535 | 1.00 | 16.67 | O |
| ATOM | 680 | N | LYS | R | 1 | 3.161 | 14.693 | 26.405 | 1.00 | 21.45 | N |
| ATOM | 681 | CA | LYS | R | 1 | 2.759 | 14.634 | 27.810 | 1.00 | 19.12 | C |
| ATOM | 682 | C | LYS | R | 1 | 4.037 | 14.567 | 28.685 | 1.00 | 18.20 | C |
| ATOM | 683 | CB | LYS | R | 1 | 1.849 | 13.397 | 28.015 | 1.00 | 20.62 | C |
| ATOM | 684 | CG | LYS | R | 1 | 1.567 | 12.973 | 29.463 | 1.00 | 30.49 | C |
| ATOM | 685 | CD | LYS | R | 1 | 0.779 | 14.001 | 30.245 | 1.00 | 33.69 | C |
| ATOM | 686 | CE | LYS | R | 1 | −0.659 | 13.590 | 30.381 | 1.00 | 40.90 | C |
| ATOM | 687 | NZ | LYS | R | 1 | −1.489 | 14.720 | 30.839 | 1.00 | 42.06 | N |
| ATOM | 688 | N | VAL | R | 2 | 4.232 | 15.536 | 29.594 | 1.00 | 13.86 | N |
| ATOM | 689 | CA | VAL | R | 2 | 5.423 | 15.562 | 30.470 | 1.00 | 12.42 | C |
| ATOM | 690 | C | VAL | R | 2 | 4.972 | 15.397 | 31.918 | 1.00 | 17.73 | C |
| ATOM | 691 | O | VAL | R | 2 | 4.408 | 16.3183 | 2.499 | 1.00 | 17.15 | O |
| ATOM | 692 | CB | VAL | R | 2 | 6.306 | 16.820 | 30.248 | 1.00 | 13.93 | C |
| ATOM | 693 | CG1 | VAL | R | 2 | 7.504 | 16.844 | 31.220 | 1.00 | 13.26 | C |
| ATOM | 694 | CG2 | VAL | R | 2 | 6.795 | 16.871 | 28.805 | 1.00 | 12.46 | C |
| ATOM | 695 | N | LYS | R | 3 | 5.252 | 14.226 | 32.488 | 1.00 | 13.32 | N |
| ATOM | 696 | CA | LYS | R | 3 | 4.825 | 13.850 | 33.844 | 1.00 | 12.63 | C |
| ATOM | 697 | C | LYS | R | 3 | 5.839 | 14.203 | 34.920 | 1.00 | 14.03 | C |
| ATOM | 698 | O | LYS | R | 3 | 7.007 | 14.408 | 34.624 | 1.00 | 11.95 | O |
| ATOM | 699 | CB | LYS | R | 3 | 4.488 | 12.342 | 33.887 | 1.00 | 13.76 | C |
| ATOM | 700 | CG | LYS | R | 3 | 3.263 | 11.960 | 33.080 | 1.00 | 24.21 | C |
| ATOM | 701 | CD | LYS | R | 3 | 2.904 | 10.524 | 33.374 | 1.00 | 35.50 | C |
| ATOM | 702 | CE | LYS | R | 3 | 1.918 | 9.953 | 32.396 | 1.00 | 50.90 | C |
| ATOM | 703 | NZ | LYS | R | 3 | 1.544 | 8.564 | 32.769 | 1.00 | 62.18 | N |
| ATOM | 704 | N | VAL | R | 4 | 5.368 | 14.304 | 36.165 | 1.00 | 12.84 | N |
| ATOM | 705 | CA | VAL | R | 4 | 6.154 | 14.681 | 37.328 | 1.00 | 13.47 | C |
| ATOM | 706 | C | VAL | R | 4 | 5.826 | 13.708 | 38.463 | 1.00 | 16.73 | C |
| ATOM | 707 | O | VAL | R | 4 | 4.658 | 13.367 | 38.643 | 1.00 | 15.53 | O |
| ATOM | 708 | CB | VAL | R | 4 | 5.810 | 16.168 | 37.685 | 1.00 | 18.24 | C |
| ATOM | 709 | CG1 | VAL | R | 4 | 6.323 | 16.568 | 39.053 | 1.00 | 17.57 | C |
| ATOM | 710 | CG2 | VAL | R | 4 | 6.342 | 17.134 | 36.628 | 1.00 | 18.00 | C |
| ATOM | 711 | N | TRP | R | 5 | 6.843 | 13.260 | 39.232 | 1.00 | 12.76 | N |
| ATOM | 712 | CA | TRP | R | 5 | 6.599 | 12.353 | 40.375 | 1.00 | 13.63 | C |
| ATOM | 713 | C | TRP | R | 5 | 7.714 | 12.412 | 41.375 | 1.00 | 15.17 | C |
| ATOM | 714 | O | TRP | R | 5 | 8.883 | 12.329 | 40.996 | 1.00 | 13.20 | O |
| ATOM | 715 | CB | TRP | R | 5 | 6.394 | 10.888 | 39.916 | 1.00 | 13.40 | C |
| ATOM | 716 | CG | TRP | R | 5 | 6.262 | 9.881 | 41.030 | 1.00 | 15.34 | C |
| ATOM | 717 | CD1 | TRP | R | 5 | 7.217 | 8.995 | 41.455 | 1.00 | 18.59 | C |
| ATOM | 718 | CD2 | TRP | R | 5 | 5.090 | 9.624 | 41.828 | 1.00 | 15.51 | C |
| ATOM | 719 | NE1 | TRP | R | 5 | 6.724 | 8.231 | 42.493 | 1.00 | 18.73 | N |
| ATOM | 720 | CE2 | TRP | R | 5 | 5.420 | 8.589 | 42.737 | 1.00 | 19.29 | C |
| ATOM | 721 | CE3 | TRP | R | 5 | 3.805 | 10.207 | 41.899 | 1.00 | 16.86 | C |
| ATOM | 722 | CZ2 | TRP | R | 5 | 4.494 | 8.069 | 43.651 | 1.00 | 18.94 | C |
| ATOM | 723 | CZ3 | TRP | R | 5 | 2.888 | 9.699 | 42.820 | 1.00 | 18.64 | C |
| ATOM | 724 | CH2 | TRP | R | 5 | 3.229 | 8.626 | 43.665 | 1.00 | 19.44 | C |
| ATOM | 725 | O | GLY | R | 6 | 6.647 | 13.027 | 45.265 | 1.00 | 14.30 | O |
| ATOM | 726 | N | GLY | R | 6 | 7.355 | 12.540 | 42.641 | 1.00 | 12.09 | N |
| ATOM | 727 | CA | GLY | R | 6 | 8.356 | 12.499 | 43.694 | 1.00 | 13.57 | C |
| ATOM | 728 | C | GLY | R | 6 | 7.847 | 13.046 | 44.994 | 1.00 | 16.55 | C |
| ATOM | 729 | O | SER | R | 7 | 10.458 | 15.371 | 46.786 | 1.00 | 20.00 | O |
| ATOM | 730 | N | SER | R | 7 | 8.763 | 13.586 | 45.775 | 1.00 | 15.06 | N |
| ATOM | 731 | CA | SER | R | 7 | 8.425 | 14.187 | 47.061 | 1.00 | 16.12 | C |
| ATOM | 732 | C | SER | R | 7 | 9.369 | 15.326 | 47.360 | 1.00 | 20.68 | C |
| ATOM | 733 | CB | SER | R | 7 | 8.418 | 13.138 | 48.173 | 1.00 | 20.30 | C |
| ATOM | 734 | OG | SER | R | 7 | 9.669 | 12.487 | 48.287 | 1.00 | 25.72 | O |
| ATOM | 735 | O | ILE | R | 8 | 10.491 | 16.607 | 50.583 | 1.00 | 17.38 | O |
| ATOM | 736 | N | ILE | R | 8 | 8.928 | 16.300 | 48.181 | 1.00 | 17.06 | N |
| ATOM | 737 | CA | ILE | R | 8 | 9.762 | 17.466 | 48.472 | 1.00 | 16.92 | C |
| ATOM | 738 | C | ILE | R | 8 | 10.831 | 17.083 | 49.499 | 1.00 | 20.52 | C |
| ATOM | 739 | CB | ILE | R | 8 | 8.908 | 18.683 | 48.916 | 1.00 | 19.67 | C |
| ATOM | 740 | CG1 | ILE | R | 8 | 7.858 | 19.044 | 47.838 | 1.00 | 19.70 | C |
| ATOM | 741 | CG2 | ILE | R | 8 | 9.794 | 19.896 | 49.297 | 1.00 | 19.27 | C |
| ATOM | 742 | CD1 | ILE | R | 8 | 6.858 | 20.164 | 48.275 | 1.00 | 25.01 | C |
| ATOM | 743 | O | LYS | R | 9 | 12.703 | 18.864 | 51.446 | 1.00 | 29.02 | O |
| ATOM | 744 | N | LYS | R | 9 | 12.113 | 17.295 | 49.160 | 1.00 | 21.01 | N |
| ATOM | 745 | CA | LYS | R | 9 | 13.227 | 16.966 | 50.070 | 1.00 | 22.87 | C |
| ATOM | 746 | C | LYS | R | 9 | 13.097 | 17.703 | 51.419 | 1.00 | 29.00 | C |
| ATOM | 747 | CB | LYS | R | 9 | 14.603 | 17.250 | 49.414 | 1.00 | 25.12 | C |
| ATOM | 748 | CG | LYS | R | 9 | 14.890 | 18.734 | 49.141 | 1.00 | 40.93 | C |
| ATOM | 749 | CD | LYS | R | 9 | 16.340 | 18.985 | 48.736 | 1.00 | 44.08 | C |
| ATOM | 750 | CE | LYS | R | 9 | 16.629 | 20.459 | 48.566 | 1.00 | 52.56 | C |
| ATOM | 751 | NZ | LYS | R | 9 | 17.818 | 20.698 | 47.704 | 1.00 | 61.31 | N |
| ATOM | 752 | O | GLY | R | 10 | 11.909 | 18.032 | 55.691 | 1.00 | 33.19 | O |
| ATOM | 753 | N | GLY | R | 10 | 13.387 | 17.004 | 52.506 | 1.00 | 27.54 | N |
| ATOM | 754 | CA | GLY | R | 10 | 13.349 | 17.574 | 53.851 | 1.00 | 27.44 | C |
| ATOM | 755 | C | GLY | R | 10 | 11.989 | 17.634 | 54.529 | 1.00 | 31.90 | C |
| ATOM | 756 | O | LEU | R | 11 | 9.536 | 14.919 | 54.012 | 1.00 | 27.67 | O |
| ATOM | 757 | N | LEU | R | 11 | 10.899 | 17.254 | 53.827 | 1.00 | 26.06 | N |
| ATOM | 758 | CA | LEU | R | 11 | 9.554 | 17.277 | 54.414 | 1.00 | 25.69 | C |
| ATOM | 759 | C | LEU | R | 11 | 8.988 | 15.873 | 54.601 | 1.00 | 21.64 | C |
| ATOM | 760 | CB | LEU | R | 11 | 8.600 | 18.144 | 53.557 | 1.00 | 25.79 | C |
| ATOM | 761 | CG | LEU | R | 11 | 9.032 | 19.603 | 53.277 | 1.00 | 29.56 | C |
| ATOM | 762 | CD1 | LEU | R | 11 | 7.944 | 20.341 | 52.522 | 1.00 | 28.67 | C |
| ATOM | 763 | CD2 | LEU | R | 11 | 9.314 | 20.378 | 54.590 | 1.00 | 31.94 | C |
| ATOM | 764 | OXT | LEU | R | 11 | 7.986 | 15.712 | 55.319 | 1.00 | 24.84 | O |
| TER | 765 | | LEU | R | 11 | | | | | | |
| HETATM | 534 | O | HOH | H | 2 | 15.969 | 21.111 | 3.194 | 1.00 | 21.24 | O |
| HETATM | 535 | O | HOH | H | 3 | 8.202 | 9.464 | 46.405 | 1.00 | 26.13 | O |
| HETATM | 536 | O | HOH | H | 4 | 2.332 | 17.528 | 29.898 | 1.00 | 18.07 | O |
| HETATM | 537 | O | HOH | H | 7 | 11.495 | 6.017 | 24.647 | 1.00 | 24.26 | O |
| HETATM | 538 | O | HOH | H | 8 | 19.707 | 15.891 | 34.532 | 1.00 | 50.79 | O |
| HETATM | 539 | O | HOH | H | 9 | 2.874 | 16.540 | 60.240 | 1.00 | 15.94 | O |
| HETATM | 540 | O | HOH | H | 10 | 7.302 | 14.394 | 21.093 | 1.00 | 19.56 | O |
| HETATM | 541 | O | HOH | H | 11 | 21.181 | 22.280 | 28.375 | 1.00 | 32.79 | O |
| HETATM | 542 | O | HOH | H | 12 | 16.164 | 22.598 | 33.451 | 1.00 | 19.82 | O |
| HETATM | 543 | O | HOH | H | 13 | 13.120 | 15.942 | 12.066 | 1.00 | 19.19 | O |
| HETATM | 544 | O | HOH | H | 14 | 15.441 | 12.095 | 37.265 | 1.00 | 27.98 | O |
| HETATM | 545 | O | HOH | H | 15 | 8.632 | 15.019 | 51.500 | 1.00 | 19.81 | O |
| HETATM | 546 | O | HOH | H | 16 | 1.326 | 12.238 | 50.021 | 1.00 | 20.76 | O |
| HETATM | 547 | O | HOH | H | 17 | 14.178 | 17.203 | 42.562 | 1.00 | 20.80 | O |
| HETATM | 548 | O | HOH | H | 18 | −3.435 | 18.806 | 46.412 | 1.00 | 38.45 | O |
| HETATM | 549 | O | HOH | H | 19 | 0.682 | 14.434 | 24.924 | 1.00 | 34.18 | O |
| HETATM | 550 | O | HOH | H | 20 | 6.765 | 9.935 | 26.132 | 1.00 | 36.05 | O |
| HETATM | 551 | O | HOH | H | 21 | −0.448 | 20.130 | 27.310 | 1.00 | 36.66 | O |
| HETATM | 552 | O | HOH | H | 22 | 8.113 | 10.696 | 15.939 | 1.00 | 36.53 | O |
| HETATM | 553 | O | HOH | H | 23 | −0.536 | 26.789 | 28.842 | 1.00 | 39.79 | O |
| HETATM | 554 | O | HOH | H | 24 | 20.177 | 25.084 | 26.407 | 1.00 | 37.59 | O |
| HETATM | 555 | O | HOH | H | 25 | 18.512 | 26.024 | 29.679 | 1.00 | 45.63 | O |
| HETATM | 556 | O | HOH | H | 26 | 10.822 | 10.425 | 42.363 | 1.00 | 29.50 | O |
| HETATM | 557 | O | HOH | H | 27 | −0.822 | 17.311 | 30.225 | 1.00 | 34.53 | O |
| HETATM | 558 | O | HOH | H | 28 | 16.949 | 29.218 | 29.469 | 1.00 | 48.83 | O |
| HETATM | 559 | O | HOH | H | 29 | 19.493 | 20.916 | 38.746 | 1.00 | 35.99 | O |
| HETATM | 560 | O | HOH | H | 30 | 15.326 | 14.117 | 48.000 | 1.00 | 35.67 | O |
| HETATM | 561 | O | HOH | H | 31 | 17.688 | 18.902 | 40.374 | 1.00 | 50.00 | O |
| HETATM | 562 | O | HOH | H | 32 | 18.658 | 23.299 | 36.777 | 1.00 | 43.87 | O |
| HETATM | 563 | O | HOH | H | 33 | −0.105 | 16.483 | 26.665 | 1.00 | 32.21 | O |
| HETATM | 564 | O | HOH | H | 34 | 12.142 | 11.906 | 16.255 | 1.00 | 36.26 | O |
| HETATM | 565 | O | HOH | H | 35 | 11.522 | 10.642 | 44.880 | 1.00 | 40.05 | O |
| HETATM | 566 | O | HOH | H | 36 | 3.750 | 13.764 | 18.778 | 1.00 | 46.14 | O |
| HETATM | 567 | O | HOH | H | 37 | 16.810 | 16.334 | 42.447 | 1.00 | 37.16 | O |
| HETATM | 568 | O | HOH | H | 38 | 1.513 | 14.230 | 63.606 | 1.00 | 38.54 | O |
| HETATM | 569 | O | HOH | H | 39 | 6.411 | 11.922 | 18.015 | 1.00 | 28.11 | O |
| HETATM | 570 | O | HOH | H | 40 | 17.143 | 26.190 | 35.705 | 1.00 | 49.47 | O |
| HETATM | 571 | O | HOH | H | 41 | 1.294 | 13.336 | 59.916 | 1.00 | 54.53 | O |
| HETATM | 572 | O | HOH | H | 42 | 0.490 | 8.479 | 29.763 | 1.00 | 41.14 | O |
| HETATM | 573 | O | HOH | H | 43 | 5.179 | 13.445 | 62.713 | 1.00 | 54.51 | O |
| HETATM | 574 | O | HOH | H | 44 | 6.288 | 12.114 | 60.683 | 1.00 | 38.54 | O |
| HETATM | 575 | O | HOH | H | 45 | −2.566 | 21.797 | 33.671 | 1.00 | 39.42 | O |
| HETATM | 576 | O | HOH | H | 46 | 14.340 | 13.513 | 8.675 | 1.00 | 33.58 | O |
| HETATM | 577 | O | HOH | H | 47 | 3.897 | 12.528 | 56.434 | 1.00 | 46.48 | O |
| HETATM | 578 | O | HOH | H | 48 | 1.875 | 7.919 | 35.441 | 1.00 | 42.84 | O |
| HETATM | 579 | O | HOH | H | 49 | −5.796 | 15.326 | 53.768 | 1.00 | 46.36 | O |
| HETATM | 580 | O | HOH | H | 50 | 10.676 | 7.846 | 41.949 | 1.00 | 39.48 | O |
| HETATM | 581 | O | HOH | H | 51 | 9.842 | 10.464 | 46.609 | 1.00 | 31.76 | O |
| HETATM | 582 | O | HOH | H | 52 | 18.833 | 24.534 | 33.281 | 1.00 | 54.91 | O |
| HETATM | 583 | O | HOH | H | 53 | −1.615 | 10.069 | 30.804 | 1.00 | 8.21 | O |
| HETATM | 584 | O | HOH | H | 54 | 3.824 | 7.901 | 50.636 | 1.00 | 21.67 | O |
| HETATM | 585 | O | HOH | H | 55 | 18.854 | 12.689 | 19.628 | 1.00 | 36.96 | O |
| HETATM | 756 | I | AIOD | I | 1 | 11.299 | 8.825 | 29.679 | 0.18 | 8.28 | I |
| HETATM | 757 | I | BIOD | I | 1 | 10.361 | 8.502 | 28.869 | 0.50 | 43.34 | I |
| HETATM | 758 | I | AIOD | I | 2 | −1.555 | 14.060 | 34.743 | 0.25 | 9.10 | I |
| HETATM | 759 | I | BIOD | I | 2 | −1.199 | 13.308 | 34.198 | 0.25 | 6.11 | I |
| HETATM | 590 | O3 | GOL | J | 1 | 14.115 | 6.943 | 15.924 | 1.00 | 44.69 | O |
| HETATM | 591 | C3 | GOL | J | 1 | 14.878 | 7.882 | 16.674 | 1.00 | 49.42 | C |
| HETATM | 592 | C2 | GOL | J | 1 | 15.397 | 8.997 | 15.797 | 1.00 | 53.37 | C |
| HETATM | 593 | O2 | GOL | J | 1 | 14.289 | 9.734 | 15.278 | 1.00 | 55.44 | O |
| HETATM | 594 | Cl | GOL | J | 1 | 16.296 | 9.928 | 16.578 | 1.00 | 54.84 | C |
| HETATM | 595 | O1 | GOL | J | 1 | 17.653 | 9.514 | 16.497 | 1.00 | 54.81 | O |
| END | | | | | | | | | | | |

### Example IV

### Other Cylindrins, from Amyloid or Amyloid-Related Proteins

Using the ABC cylindrin as the profiled structure, and following methods disclosed herein, the inventors have determined cylindrin-forming sequences of a variety of representative amyloid or amyloid-related proteins. Table 7 shows some of these sequences, as well as mutant forms of the peptides which have been used to characterize the cylindrins. Shown are sequences from alphaB crystallin (ABC), Amyloid beta peptide (Abeta, or Aβ) of Alzheimer's disease, Islet amyloid polypeptide (IAPP, associated with diabetes type 2), Prion protein (PrP), Superoxide dismutase1 (SOD1), α-Synuclein (associated with Parkinson's disease), Tau and TDP43. In this table, TR means tandem repeat; Arctic, E22del, and Iowa are various mutant Abeta sequences; Capped means an acetyl group (CH₃—CO—) is on the N-terminus, and an amino group (—NH₂) on the C-terminus to make them more protein-like (no terminal charges). The upper case letters (for example, in the SOD1 segments) represent sequences that are important for the formation of a cylindrin. The lower case letters represent looped out regions (intervening sequences) between the sequences involved in the formation of the cylindrin. The loops are important for the ability of the strands of the cylindrin to fold back upon one another to form the antiparallel strands of the cylindrin.

Skilled workers, using the ABC cylindrin structure, the SOD1 cylindrin structure, or others, can readily identify cylindrin-forming sequences from any amyloid or amyloid-related protein of interest, using the methods described herein. Using conventional methods, such as those described herein, cylindrin-forming segments are synthesized, allowed to aggregate to form cylindrins, shown to be toxic, crystallized and their 3D structures determined, and/or used to identify agents which inhibit or reduce cylindrin-mediated activities, such as cytotoxicity.

TABLE 7

| | | |
|---|---|---|
| | SEQ | |
| Construct | ID NO: | Protein Sequence |

αB crystallin (ABS)

| | | |
|---|---|---|
| ABC K11V | 3 | KVKLGDVIEV |
| ABC K11V + | 6 | KLKVLGDVIEV |
| ABC K11V-Br2 | 4 | KBrKVLGDVIEV |
| ABC K11V-Br8 | 5 | KVKVLGDBrIEV |
| ABC K11V + | 13 | gKLKVLGDVIEV |
| ABC K11V-TR | 7 | gKLKVLGDVIEVggKLKVLGDVIEV |
| ABC K11V-TR_02 | 14 | gKLKVLGDVIEVpgKLKVLGDVIEV |
| ABC K11V-TR_03 | 15 | gKLKVLGDVIEVggggKLKVLGDVIEV |
| ABC K11V-TR_04 | 16 | KLKVLGDVIEVggKLKVLGDVIEV |
| ABC K11V-TR_05 | 17 | gKVKVLGDVIEVggKVKVLGDVIEV |
| ABC K11V(L5N)-TR | 18 | gKLKVNGDVIEVggKLKVNGDVIEV |
| ABC K11V(V4W)-TR | 9 | gKLKWLGDVIEVggKLKWLGDVIEV |
| ABC K11V(G6P)-TR | 19 | gKLKVLPDVIEVggKLKVLPDVIEV |
| ABC K11V(R2D2)-TR | 20 | gKLKRLGDDIEVggKLKDLGDRIEV |

Amyloid β (Aβ)

| | | |
|---|---|---|
| Aβ(24-34)-TR | 21 | gVGSNKGAIIGLggVGSNKGAIIGL |
| Aβ(26-36)-TR | 22 | gSNKGAIIGLMVggSNKGAIIGLMV |
| Aβ(28-38)-TR | 23 | gKGAIIGLMVGGggKGAIIGLMVGG |
| Aβ(30-40)-TR | 24 | gAIIGLMVGGVVggAIIGLMVGGVV |
| Aβ(32-42)-TR | 25 | gIGLMVGGVVIAggIGLMVGGVVIA |
| Aβ(28-42)-TR | 26 | gKGAIIGLMVGGVVIAggKGAIIGLMVGGVVIA |
| Aβ(17-27-arctic)-TR | 27 | gLVFFAGDVGSNggLVFFAGDVGSN |
| Aβ(17-38-arctic) | 28 | gLVFFAGDVGSNKGAIIGLMVGG |
| Aβ(17-42-arctic) | 29 | gLVFFAGDVGSNkgaiIGLMVGGVVIA |
| Aβ(16-38-E22del) | 30 | gKLVFFADVGSNKGAIIGLMVGG |
| Aβ(16-42-E22del) | 31 | gKLVFFADVGSNkgaiIGLMVGGVVIA |
| Aβ(16-38-Iowa) | 32 | gKLVFFAENVGSnKGAIIGLMVGG |
| Aβ(16-42-Iowa) | 33 | gKLVFFAENVGSnkgaiIGLMVGGVVIA |
| Aβ(30-42)-Tandem3-GG | 34 | gAIIGLMVGGVVIAgpAIIGLMVGGVVIAgpAIIGLMVGGVVIA |
| Aβ(30-42)G38P-Tandem3-GP | 35 | gAIIGLMVGPVVIAgpAIIGLMVGPVVIAgpAIIGLMVGPVVIA |
| Aβ(30-42)-Tandem6 | 36 | gAIIGLMVGGVVIAgpAIIGLMVGGVVIAgpAIIGLMVGGVVIAgpAIIGLMVGG |
| | | VVIAgpAIIGLMVGGVVIAgpAIIGLMVGGVVIA |
| Aβ(21-30) capped | 37 | Ac-AGDVGSNKGA-NH2 |
| Aβ(21-30)-TR capped | 38 | Ac-AGDVGSNKGAggAGDVGSNKGA-NH2 |
| Aβ(24-34) capped | 39 | Ac-VGSNKGAIIGL-NH2 |
| Aβ(24-34)-TR capped | 40 | Ac-VGSNKGAIIGLggVGSNKGAIIGL-NH2 |
| Aβ(25-35) capped | 41 | Ac-GSNKGAIIGLM-NH2 |
| Aβ(25-35)-TR capped | 42 | Ac-GSNKGAIIGLMggGSNKGAIIGLM-NH2 |
| Aβ(25-35) uncapped | 43 | GSNKGAIIGLM |
| Aβ(26-36) capped | 44 | Ac-SNKGAIIGLMV-NH2 |
| Aβ G29V | 45 | DAEFRHDSGYEVHHQKLVFFAEDVGSNKVAIIGLMVGGVVIA (42) |
| Aβ G33V | 46 | DAEFRHDSGYEVHHQKLVFFAEDVGSNKGAIIVLMVGGVVIA (42) |
| Aβ G37V | 47 | DAEFRHDSGYEVHHQKLVFFAEDVGSNKGAIIGLMVVGVVIA (42) |
| Aβ G38V | 48 | DAEFRHDSGYEVHHQKLVFFAEDVGSNKGAIIGLMVGVVVIA (42) |

Islet amyloid polypeptide (IAPP)

| | | |
|---|---|---|
| IAPP (19-29) TR | 49 | gSSNNFGAILSSggSSNNFGAILSS |
| IAPP | 50 | KCNTATCATQRLANFLVHSSNNFGAILSSTNVGSNTY (37) |
| IAPP G24V | 51 | KCNTATCATQRLANFLVHSSNNFVAILSSTNVGSNTY (37) |
| IAPP G33V | 52 | KCNTATCATQRLANFLVHSSNNFGAILSSTNVVSNTY (37) |
| IAPP G24V/G33V | 53 | KCNTATCATQRLANFLVHSSNNFVAILSSTNVVSNTY (37) |

Prion protein (PrP)

| | | |
|---|---|---|
| PrP(116-126)-TR | 54 | gAAAGAVVGGLGggAAAGAVVGGLG |
| PrP(120-130)-TR | 55 | gAVVGGLGGYMLggAVVGGLGGYML |
| PrP(124-134)-TR | 56 | GLGGYMLGSAMggGLGGYMLGSAM |
| PrP(190-200)-TR | 57 | gTTTTKGENFTEggTTTTKGENFTE |
| PrP(109-132)-TR | 58 | gMKHMAGAAAAGavVGGLGGYMLGS |
| PrP(114-136)-TR | 59 | GAAAAGAVVGGlGGYMLGSAMSR |

Superoxide dismutase 1 (SOD-1)

| | | |
|---|---|---|
| SOD1(29-39) | 60 | PVKVWGSIKGL |
| SOD1(29-39) P29K | 61 | KVKVWGSIKGL |
| SOD1(29-39)P29K-TR | | gKVKVWGSIKGLggKVKVWGSIKGL |
| SOD1(31-41)-TR | 62 | gKVWGSIKGLTEggKVWGSIKGLTE |
| SOD1(33-43)-TR | 63 | gWGSIKGLTEGLggWGSIKGLTEGL |
| SOD1 | 64 | |
| SOD1 G34V | 65 | . . . (31) KVWVSIKGLTE (41) . . . |
| (G33V in literature) | | |
| SOD1 G38V | 66 | . . . (31) KVWGSIKVLTE (41) . . . |
| (G37V in literature) | | |
| SOD1 G94V | 67 | |
| (G93A in literature) | | |
| SOD1 G34V/G94A | 68 | |
| (G33V/G93A in literature) | | |
| SOD1 A5V | | |
| (A4V in literature) | | |
| SOD1 A5V/G34V | | |
| (A4V/G33V in literature) | | |

α-Synuclein

| | | |
|---|---|---|
| α-Syn(63-73)-TR | 69 | gVTNVGGAVVTGggVTNVGGAVVTG |
| α-Syn(68-78)-TR | 70 | GAVVTGVTAVAggGAVVTGVTAVA |
| α-Syn(46-73) | 71 | gEGVVHGVATVAektkeqVTNVGGAVVTG |
| α-Syn(63-91) | 72 | gVTNVGGAVVTGvtavaqkTVEGAGSIAAA |
| α-Syn(63-91)linkerΔng | 73 | gVTNVGGAVVTGngTVEGAGSIAAA |
| α-Syn(68-98) | 74 | GAVVTGVTAVAqktvegagsIAAATGFVKKD |
| α-Syn(88-98) | 75 | IAAATGFVKKD |

Tau

| | | |
|---|---|---|
| Tau(23-33)-TR | 76 | gRKDQGGYTMHQggRKDQGGYTMHQ |
| Tau(32-42)-TR | 77 | gHQDQEGDTDAGggHQDQEGDTDAG |
| Tau(115-125)-TR | 78 | gEDEAAGHVTQAggEDEAAGHVTQA |
| Tau(142-152)-TR | 79 | gAKGADGKTKIAggAKGADGKTKIA |
| Tau(142-152)A152T-TR | 80 | gAKGADGKTKITggAKGADGKTKIT |
| Tau(142-152) | 81 | AKGADGKTKIA |
| Tau(142-152) A152T | 82 | AKGADGKTKIT |
| Tau(396-406)-TR | 83 | gSPVVSGDTSPRggSPVVSGDTSPR |
| Tau(410-420)-TR | 84 | gNVSSTGSIDMVggNVSSTGSIDMV |

TDP43

| | | |
|---|---|---|
| TDP43(247-257) | 85 | DLIIKGISVHI |
| TDP43(247-258) | 86 | DLIIKGISVHIE |

TDP43

| | | |
|---|---|---|
| TDP 321-343 | 87 | gAMMAAAQAALQsSWGMMGMLASQ |
| TDP 321-343 Q331K | 88 | gAMMAAAQAALKsSWGMMGMLASQ |
| TDP 321-343 M337V | 89 | gAMMAAAQAALQsSWGMVGMLASQ |
| TDP 333&323 | 90 | gSWGMMGMLASQggMAAAQAALQSS |
| TDP 333&323 | 91 | gSWGMMGMLASQggMAAAQAALKSS |
| TDP 333&323 | 92 | gSWGMVGMLASQggMAAAQAALQSS |
| TDP 304-333 | 93 | gGSNMGGGMNFGafsinpamMAAAQAALQSS |
| TDP 304-333 Q331K | 94 | gGSNMGGGMNFGafsinpamMAAAQAALKSS |
| TDP 304-333 A315T | 95 | gGSNMGGGMNFGtfsinpamMAAAQAALQSS |
| TDP 309-340 | 96 | gGGMNFGAFSINpammaaaqaaLQSSWGMMGML |
| TDP 309-340 Q331K | 97 | gGGMNFGAFSINpammaaaqaaLKSSWGMMGML |
| TDP 309-340 M337V | 98 | gGGMNFGAFSINpammaaaqaaLQSSWGMVGML |
| TDP 309-340 A315T | 99 | gGGMNFGTFSINpammaaaqaaLQSSWGMMGML |
| TDP 333-362 | 100 | gSWGMMGMLASQqnqsgpsgNNQNQGNMQRE |
| TDP 333-362 M337V | 101 | gSWGMVGMLASQqnqsgpsgNNQNQGNMQRE |
| TDP 333-362 N345K | 102 | gSWGMMGMLASQqkqsgpsgNNQNQGNMQRE |
| TDP 333 | 103 | gSWGMMGMLASQggSWGMMGMLASQ |
| TDP 333 M337V | 104 | gSWGMVGMLASQggSWGMVGMLASQ |
| TDP 330 | 105 | gLQSSWGMMGMLggLWSSWGMMGML |
| TDP 330 Q331K | 106 | gLKSSWGMMGMLggLKSSWGMMGML |
| TDP 330 M337V | 107 | gLQSSWGMVGMLggLQSSWGMVGML |
| TDP D11I 247 | 108 | gDLIIKGISVHIggDKIIKGISVHI |

### REFERENCES

1. B. Caughey, P. T. Lansbury, Annu Rev Neurosci 26, 267 (2003).
2. W. F. Xue et al., J Biol Chem 284, 34272 (Dec. 4, 2009).
3. R. Kodali, R. Wetzel, Curr Opin Struct Biol 17, 48 (February, 2007).
4. M. D. Kirkitadze, G. Bitan, D. B. Teplow, J Neurosci Res 69, 567 (Sep. 1, 2002).
5. R. Kayed et al., Science 300, 486 (Apr. 18, 2003).
6. G. Bitan, E. A. Fradinger, S. M. Spring, D. B. Teplow, Amyloid 12, 88 (June, 2005).
7. C. G. Glabe, J Biol Chem 283, 29639 (Oct. 31, 2008).
8. F. Chiti, C. M. Dobson, Annu Rev Biochem 75, 333 (2006).
9. F. Chiti, C. M. Dobson, Nat Chem Biol 5, 15 (January, 2009).
10. J. C. Rochet, P. T. Lansbury, Jr., Curr Opin Struct Biol 10, 60 (February, 2000).
11. R. Kayed et al., Mol Neurodegener 2, 18 (2007).
12. J. Horwitz, Proc Natl Acad Sci USA 89, 10449 (Nov. 1, 1992).
13. S. Jehle et al., Proceedings of the National Academy of Sciences of the United
States of America 108, 6409 (Apr. 19, 2011).
14. H. Ecroyd, J. A. Carver, Cell Mol Life Sci 66, 62 (January, 2009).
15. S. Meehan et al., J Mol Biol 372, 470 (Sep. 14, 2007).
16. A. Laganowsky et al., Protein Sci 19, 1031 (May, 2010).
17. M. J. Thompson et al., Proc Natl Acad Sci USA 103, 4074 (Mar. 14, 2006).
18. L. Goldschmidt, P. K. Teng, R. Riek, D. Eisenberg, Proc Natl Acad Sci USA 107, 3487 (Feb. 23, 2010).
19. M. R. Sawaya et al., Nature 447, 453 (May 24, 2007).
20. F. Sarsoza et al., Acta Neuropathol 118, 505 (October, 2009).
21. A. V. Kajava, U. Baxa, A. C. Steven, The FASEB journal: official publication of the Federation of American Societies for Experimental Biology 24, 1311 (May, 2010).
22. J. Greenwald, R. Riek, Structure 18, 1244 (Oct. 13, 2010).
23. B. M. Austen et al., Biochemistry 47, 1984 (Feb. 19, 2008).
24. P. Picone et al., Biophys J 96, 4200 (May 20, 2009).
25. M. F. Engel et al., Proc Natl Acad Sci USA 105, 6033 (Apr. 22, 2008).
26. L. Khemtemourian, M. F. Engel, R. M. Liskamp, J. W. Hoppener, J. A. Killian, Biochim Biophys Acta 1798, 1805 (September, 2010).
27. P. M. Pryciak, Conway, J. D., Eiserling, F. A., and D. Eisenberg, in Protein Structure, Folding, and Design, D. L. Oxender, Ed. (Alan R. Liss, New York, 1986), vol. 39, pp.
243-246.
28. F. R. Salemme, D. W. Weatherford, J Mol Biol 146, 119 (Feb. 15, 1981).
29. F. R. Salemme, D. W. Weatherford, J Mol Biol 146, 101 (Feb. 15, 1981).
30. Y. Shafrir, S. Durell, N. Arispe, H. R. Guy, Proteins 78, 3473 (December, 2010).
31. J. D. Conway, UCLA (1986).
32. R. Nelson et al., Nature 435, 773 (Jun. 9, 2005).
33. A. G. Murzin, A. M. Lesk, C. Chothia, J Mol Biol 236, 1369 (Mar. 11, 1994).
34. T. L. Benzinger et al., Proc Natl Acad Sci USA 95, 13407 (Nov. 10, 1998).
35. R. Tycko, Annu Rev Phys Chem 62, 279 (May, 2011).
36. N. B. Last, E. Rhoades, A. D. Miranker, Proc Natl Acad Sci USA 108, 9460 (Jun. 7, 2011).
37. J. W. Wu et al., J Biol Chem 285, 6071 (Feb. 26, 2010).
38. K. Kar, M. Jayaraman, B. Sahoo, R. Kodali, R. Wetzel, Nat Struct Mol Biol 18, 328 (March, 2011).
39. S. Chimon et al., Nat Struct Mol Biol, (Dec. 2, 2007).
40. K. Ono, M. M. Condron, D. B. Teplow, Proc Natl Acad Sci USA 106, 14745 (Sep. 1, 2009).
41. J. L. Tomic, A. Pensalfini, E. Head, C. G. Glabe, Neurobiol Dis 35, 352 (September, 2009).
42. Y. Yoshiike, R. Kayed, S. C. Milton, A. Takashima, C. G. Glabe, Neuromolecular Med 9, 270 (2007).
43. S. L. Bernstein et al., Nat Chem 1, 326 (July, 2009).
44. M. Ahmed et al., Nat Struct Mol Biol 17, 561 (May, 2010).
45. G. Bitan, S. S. Vollers, D. B. Teplow, J Biol Chem 278, 34882 (Sep. 12, 2003).
46. H. Jang et al., J Mol Biol 404, 917 (Dec. 17, 2010).
47. H. A. Lashuel, D. Hartley, B. M. Petre, T. Walz, P. T. Lansbury, Jr., Nature 418, 291 (Jul. 18, 2002).
48. C. Liu et al., J Am Chem Soc 133, 6736 (May 4, 2011).
49. V. A. Streltsov, J. N. Varghese, C. L. Masters, S. D. Nuttall, J Neurosci 31, 1419 (Jan. 26, 2011).
50. F. C. Dehle, H. Ecroyd, I. F. Musgrave, J. A. Carver, Cell Stress Chaperones 15, 1013 (November, 2010).

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever. The entire disclosure of all applications, patents, and publications cited above, including U.S. Provisional Applications 61/588,478, filed Jan. 19, 2012, and 61/590,085, filed Jan. 24, 2012, and in the figures are hereby incorporated in their entirety by reference, particularly with regard to the information for which they are cited.

1. A cylindrin, which is a non-covalent assembly of substantially identical chains of an amyloid or amyloid-related protein,
wherein each chain has a length of about 10-100 amino acid residues and comprises
a single copy of a cylindrin-forming segment, or
tandem adjacent copies of a cylindrin-forming segment, optionally separated by spacers, or
adjacent copies of a first cylindrin-forming segment and a second complementary segment of the first cylindrin-forming segment, optionally separated by spacers,
wherein at least about ⅔ of the amino acid residues in the chain are cylindrin-forming segments,
wherein the cylindrin is a curved beta sheet formed from anti-parallel out-of-register extended protein strands, which is substantially filled with packed side chains.

2. The cylindrin of claim 1, which is a cylindrical barrel formed from anti-parallel extended protein strands.

3. The cylindrin of claim 1, which is an antiparallel beta-sheet corkscrew.

4. The cylindrin of claim 1, wherein the amyloid or amyloid-related protein is beta amyloid, tau, amylin, Prion protein (PrP), Superoxide dismutase) (SOD)), TAR DNA-binding protein 43 (TDP-43), RNA-binding protein FUS (Fused in Sarcoma), or alpha-synuclein.

5. The cylindrin of claim 1, wherein the amyloid or amyloid-related protein is associated with a neurodegenerative disease or condition.

6. The cylindrin of claim 1, wherein the amyloid or amyloid-related protein is alphaB crystallin (ABC), Abeta, or superoxide dismutase 1 (SOD)).

7. The cylindrin of claim 1, wherein the cylindrin-forming segment or the chain comprising cylindrin-forming segments is one of the peptides listed in Table 7.

8. The cylindrin of claim 1, wherein each chain comprises a Gly residue which occupies a central location in the cylindrin-forming segment and points toward the interior of the cylindrin (the interior of the curvature).

9. The cylindrin of claim 1, which is a toxic agent for a neurodegenerative disease.

10. The cylindrin of claim 1, which is detectably labeled.

11. A nucleic acid encoding a chain of an amyloid or amyloid-related protein comprising
a single copy of a cylindrin-forming segment, or
tandem adjacent copies of a cylindrin-forming segment, optionally separated by spacers, or
adjacent copies of a first cylindrin-forming segment and a second complementary segment of the first cylindrin-forming segment, optionally separated by spacers.

12. An expression vector comprising the nucleic acid of 11, operably linked to an expression control sequence.

13. A cell comprising the expression vector of claim 12.

14. A method for making a cylindrin of claim 1, comprising
identifying a cylindrin-forming segment from an amyloid or amyloid-related protein of interest, by using the structure of a known cylindrin as a profiled structure in a method of 3D profiling,
synthesizing copies of the cylindrin-forming segment, and
allowing the copies to form oligomers,
thereby forming a cylindrin.

15. The method of claim 14, wherein the known cylindrin structure is of ABC.

16. (canceled)

17. The method of claim 15, wherein the atomic coordinates of the structure are shown in Table 5.

18. The method of claim 14, wherein the known cylindrin structure is of SOD1.

19. (canceled)

20. The method of claim 18, wherein the atomic coordinates of the structure are shown in Table 6.

21. The method of claim 14, further comprising testing whether the cylindrin is toxic to a cell.

22. The method of claim 14, further comprising crystallizing and determining the 3D structure of the cylindrin.

23. A method for identifying a putative agent that inhibits or reduces cylindrin-mediated toxicity, comprising
contacting cells with the cytotoxic cylindrin of claim 1 and with a putative inhibitory agent, and
determining the viability of the cells which were contacted with the putative agent compared to the viability of control cells which were not contacted with the putative inhibitory agent,
wherein a putative agent that results in a statistically significantly greater viability of the cells that were contacted with the putative agent compared to the cells which were not contacted with the putative agent is a candidate for an agent that inhibits cylindrin-mediated toxicity.

24. A computer-readable medium, providing the structural representation of a cylindrin of claim 1.

25. A kit, comprising reagents for making and/or characterizing a cylindrin of claim 1, or for identifying a putative agent that inhibits or reduces cell toxicity of a cylindrin of claim 1.
